(19) United States
(12) Patent Application Publication
(10) Pub. No.: US 2025/0313835 A1
(43) Pub. Date: Oct. 9, 2025

(54) MICELLAR NANOPARTICLES AND USES THEREOF

(71) Applicant: Biorchestra Co., Ltd., Daejeon (KR)

(72) Inventors: Jin-Hyeob RYU, Daejeon (KR); Yu Na LIM, Daejeon (KR); Hyun Su MIN, Daejeon (KR); Hyewon PARK, Daejeon (KR)

(73) Assignee: Biorchestra Co., Ltd., Daejeon (KR)

(21) Appl. No.: 18/881,645

(22) PCT Filed: Jul. 6, 2023
(86) PCT No.: PCT/IB2023/057015

§ 371 Date: Jan. 6, 2025

Related U.S. Application Data
(60) Provisional application No. 63/367,907, filed on Jul. 7, 2022.

(51) Int. Cl. C12N 15/113; A61K 9/107; A61K 47/54; A61K 47/60; A61K 47/64; A61K 47/69; C12N 15/88

(52) U.S. Cl. CPC C12N 15/113; A61K 9/107; A61K 47/545; A61K 47/60; A61K 47/6455; A61K 47/6907; C12N 15/88; C12N 2310/11

(57) ABSTRACT

The present disclosure includes cationic carrier units comprising (i) a water-soluble polymer, (ii) a positively charged carrier, (iii) a hydrophobic moiety, and (iv) a crosslinking moiety, wherein when the cationic carrier unit is mixed with an anionic payload (e.g., an antisense oligonucleotide) that electrostatically interacts with the cationic carrier unit, the resulting composition self-organizes into a micelle encapsulating the anionic payload in its core. The cationic carrier units can also comprise a tissue specific targeting moiety, which would be displayed on the surface of the micelle. The disclosure also includes micelles comprising the cationic carrier units of the disclosure, methods of manufacture of cationic carrier units and micelles, pharmaceutical compositions comprising the micelles, and also methods of treating diseases or conditions comprising administering the micelles to a subject in need thereof.

### CROSS REFERENCE TO A RELATED APPLICATION

This PCT application claims the benefit of the filing date of U.S. Provisional Patent Application No. 63/367,907, filed Jul. 7, 2022; which is incorporated herein by reference in its entirety.

### FIELD

The present disclosure provides cationic carrier units and micelle systems, which can be used to deliver anionic payloads (e.g., oligonucleotides) across physiological permeation barriers, e.g., the brain blood barrier.

### BACKGROUND ART

There are certain barriers present into the body, which restrict the permeability of the drug through the membrane. Thus, only selective substances can pass through this type of membranes. Some important and specialized physiological barrier are the blood brain barrier and the cell membrane. The blood-brain barrier (BBB) is a highly selective semipermeable border that separates the circulating blood from the brain and extracellular fluid in the central nervous system (CNS). The blood-brain barrier is formed by endothelial cells of the capillary wall, astrocyte end-feet ensheathing the capillary, and pericytes embedded in the capillary basement membrane. This system allows the passage of water, some gases, and lipid-soluble molecules by passive diffusion, as well as the selective transport of molecules such as glucose and amino acids that are crucial to neural function.

The blood-brain barrier restricts the passage of pathogens, the diffusion of solutes in the blood, and large or hydrophilic molecules into the cerebrospinal fluid (CSF), while allowing the diffusion of O₂, CO₂, hydrophobic molecules (e.g., hormones), and small polar molecules (Johansen et al., (2017) Journal of Cerebral Blood Flow and Metabolism. Epub (4): 659-668). The BBB excludes from the brain almost 100% of large-molecule neurotherapeutics and more than 98% of all molecule drugs. Daneman & Prat (2015) “The Blood Brain Barrier” Cold Spring Harbor Perspectives in Biology 7 (1):a020412. Overcoming the difficulty of delivering therapeutic agents to specific regions of the brain represents a major challenge to treatment of most brain disorders. Thus, therapeutic molecules that might otherwise be effective in diagnosis and therapy do not cross the BBB in adequate amounts.

Intracellular targeting is also often challenging, because to reach the cytosol, exogenous molecules must first traverse the cell membrane. The cell membrane is selectively permeable to non-polar therapeutic agents, which are lipid soluble and can pass through the cell membrane. On the other hand, highly charged therapeutic agents such as oligonucleotides are effectively excluded by the cell membrane.

Polynucleotides do not readily permeate the cellular membrane due to the charge repulsion between the negatively charged membrane and the high negative charge on the polynucleotide. As a result, polynucleotides have poor bioavailability and uptake into cells, typically less than 1% (Dheur et al, Nucleic Acid Drug Dev., 9:522 (1999); Park et al, J Controlled Release, 93:188 (2003)). Since most polynucleotides are generally above 5,000 Da, they cannot readily diffuse through cellular membranes and uptake into cells is limited primarily to pinocytotic or endocytotic processes. Once inside the cell, polynucleotides can accumulate in lysosomal compartments, limiting their access to the cytoplasm or the nucleus. Parenterally administered polynucleotides are also highly susceptible to rapid nuclease degradation both inside and outside the cytoplasm. Studies show rapid degradation of polynucleotides in blood after i.v. administration, with a half-life of about 30 minutes (Geary et al, J. Pharmacol. Exp. Ther. 296:890-897 (2001)).

Thus, the problems facing the delivery of polynucleotide, e.g., antisense oligonucleotide, can roughly be divided into two parts. First, the therapeutic polynucleotide must be formulated in such a way that it can be delivered to the cytoplasm and second, the polynucleotide must reach the cell nucleus intact and fully functional. Despite the advances in application of oligonucleotides and oligonucleotide analogs as therapeutics, the need exists for delivery systems providing improved pharmacological properties, e.g., serum stability, delivery to the right organ, tissue, or cell, and transmembrane delivery.

Efforts aimed at improving the transmembrane delivery of nucleic acids and oligonucleotides have utilized protein carriers, antibody carriers, liposomal delivery systems, electroporation, direct injection, cell fusion, viral vectors, and calcium phosphate-mediated transformation. However, many of these techniques are limited by the types of cells in which transmembrane transport is enabled and by the conditions needed for achieving such transport. Accordingly, there is a need for delivery systems that can selectively direct charged therapeutic agents (e.g., antisense oligonucleotides such as antimirs) to specific target cells or tissues, and across permeation barriers (e.g., the plasma membrane or the BBB), while improving serum stability and/or resistance to endogenous lytic enzymes (e.g., RNases).

### BRIEF SUMMARY

The present disclosure provides a cationic carrier unit comprising

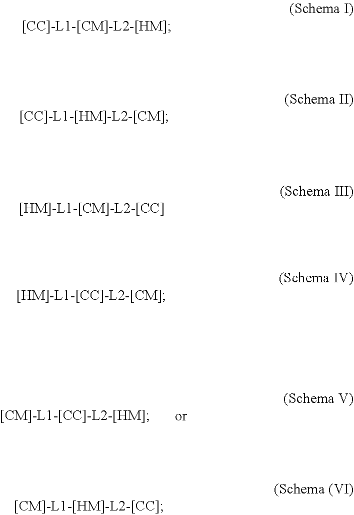

wherein
CC is a positively charged carrier moiety;
CM is a crosslinking moiety;
HM is a hydrophobic moiety, which comprises a nicotinic acid derivative; and,
L1 and L2 are independently optional linkers, and
wherein the number of HM is less than about 50% relative to [CC] and [CM].

In some aspects, the nicotinic acid derivative comprises an electron donating group or an electron withdrawing group. In some aspects, the nicotinic acid derivative comprises 2-methylnicotinic acid (Me-NA), 2-methoxynicotinic acid (MeO-NA), or any combination thereof. In some aspects, the nicotinic acid derivative comprises 2-methylnicotinic acid (Me-NA). In some aspects, the nicotinic acid derivative comprises 2-methoxynicotinic acid (MeO-NA).

In some aspects, the nicotinic acid derivative comprises:

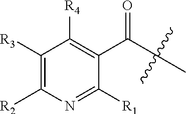

wherein each of R₁, R₂, R₃, and R₄ is an electron donating group.

In some aspects, the nicotinic acid derivative comprises:

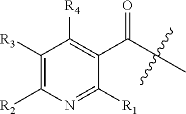

wherein each of R₁, R₂, R₃, and R₄ is an electron withdrawing group.

In some aspects, the number of HM is less than about 45%, about 40%, about 35%, about 30%, about 25%, about 20%, about 15%, about 10%, about 9%, about 8%, about 7%, about 6%, about 5%, about 4%, about 3%, about 2%, or about 1% relative to [CC] and [CM]. In some aspects, the number of HM is between about 50% and about 1%, about 50% and about 5%, about 50% and about 10%, about 50% and about 15%, about 50% and about 20%, about 50% and about 25%, about 50% and about 30%, about 50% and about 35%, about 50% and about 40%, about 50% and about 45%, about 45% and about 1%, about 45% and about 5%, about 45% and about 10%, about 45% and about 15%, about 45% and about 20%, about 45% and about 25%, about 45% and about 30%, about 45% and about 35%, about 45% and about 40%, about 40% and 1%, about 50% and about 5%, about 40% and about 10%, about 40% and about 15%, about 40% and about 20%, about 40% and about 25%, about 40% and about 30%, about 40% and about 35%, about 35% and about 1%, about 35% and about 5%, about 35% and about 10%, about 35% and about 15%, about 35% and about 20%, about 35% and about 25%, about 35% and about 30%, about 30% and about 1%, about 30% and about 5%, about 30% and about 10%, about 30% and about 15%, about 30 and about 20%, about 30% and about 25%, about 25% and about 1%, about 25% and about 5%, about 25% and about 10%, about 25% and about 15%, about 25% and about 20%, about 20% and about 1%, about 20% and about 5%, about 20% and about 10%, about 20% and about 15%, about 15% and about 1%, about 15% and about 5%, about 15% and about 10%, about 10% to about 1%, about 10% to about 5%, or about 5% to about 1% relative to [CC] and [CM]. In some aspects, the number of HM is between about 50% and about 40%, about 40% and about 30%, about 30% and about 20%, about 20% and about 10%, about 10% and about 5%, and about 5% and about 1%. In some aspects, the number of HM is about 50%, about 45%, about 40%, about 35%, about 30%, about 25%, about 20%, about 15%, about 10%, about 5%, or about 1%.

In some aspects, the cationic carrier unit is capable of interacting with an anionic payload. In some aspects, the anionic payload comprises a nucleotide sequence that is less than 200 nucleotides in length. In some aspects, the anionic payload comprises a nucleotide sequence that is less than about 150, about 140, about 130, about 120, about 110, about 100, about 90, about 80, about 70, about 60, about 50, about 40, about 30, about 25, about 24, about 23, about 22, about 21, about 20, about 19, about 18, about 17, about 16, about 15, about 14, about 13, about 12, about 11, or about 10 nucleotides in length. In some aspects, the anionic payload comprises a nucleotide sequence from about 30 to about 10, from about 25 to about 11, from about 30 to about 15, from about 25 to about 15, from about 24 to about 15, or from about 23 to about 15 nucleotides in length. In some aspects, the anionic payload comprises a nucleotide sequence having about 30, about 29, about 28, about 27, about 26, about 25, about 24, about 23, about 22, about 21, about 20, about 19, about 18, about 17, about 16, about 15, about 14, or about 13 nucleotides in length. In some aspects, the anionic payload comprises a nucleotide sequence having about 22 nucleotides in length. In some aspects, the anionic paylod comprises mRNA, miRNA, miRNA sponge, tough decoy miRNA, antimir, small RNA, IRNA, siRNA, shRNA, gDNA, cDNA, pDNA, PNA, BNA, antisense oligonucleotide (ASO), aptamer, cyclic dinucleotide, or any combination thereof.

In some aspects, the cationic carrier unit further comprises a water-soluble polymer (WP). In some aspects, the water-soluble polymer is attached to [CC], [HM], or [CM]. In some aspects, the water-soluble polymer is attached to the N terminus of [CC], [HM], or [CM]. In some aspects, the water-soluble polymer is attached to the C terminus of [CC], [HM], or [CM]. In some aspects, the cationic carrier unit comprises:

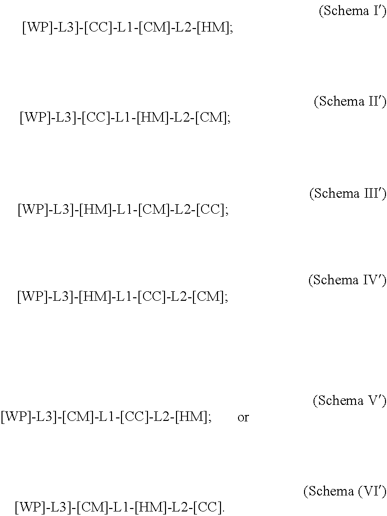

In some aspects, the water-soluble polymer comprises poly(alkylene glycols), poly(oxyethylated polyol), poly(olefinic alcohol), poly(vinylpyrrolidone), poly(hydroxyalkylmethacrylamide), poly(hydroxyalkylmethacrylate), poly(saccharides), poly(a-hydroxy acid), poly(vinyl alcohol), polyglycerol, polyphosphazene, polyoxazolines (“POZ”) poly(N-acryloylmorpholine), or any combinations thereof. In some aspects, the water-soluble polymer comprises polyethylene glycol (“PEG”), polyglycerol, or poly(propylene glycol) (“PPG”). In some aspects, the water-soluble polymer comprises:

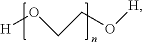

wherein n is 1-1000.

In some aspects, n is at least about 110, at least about 111, at least about 112, at least about 113, at least about 114, at least about 115, at least about 116, at least about 117, at least about 118, at least about 119, at least about 120, at least about 121, at least about 122, at least about 123, at least about 124, at least about 125, at least about 126, at least about 127, at least about 128, at least about 129, at least about 130, at least about 131, at least about 132, at least about 133, at least about 134, at least about 135, at least about 136, at least about 137, at least about 138, at least about 139, at least about 140, or at least about 141. In some aspects, n is about 80 to about 90, about 90 to about 100, about 100 to about 110, about 110 to about 120, about 120 to about 130, about 140 to about 150, or about 150 to about 160.

In some aspects, the water-soluble polymer is linear, branched, or dendritic.

In some aspects, the cationic carrier moiety comprises one or more basic amino acids. In some aspects, the cationic carrier moiety comprises at least about three, at least about four, at least about five, at least about six, at least about seven, at least about eight, at least about nine, at least about ten, at least about 11, at least about 12, at least about 13, at least about 14, at last about 15, at least about 16, at least about 17, at least about 18, at least about 19, at least about 20, at least about 21, at least about 22, at least about 23, at least about 24, at least about 25, at least about 26, at least about 27, at least about 28, at least about 29, at least about 30, at least about 31, at least about 32, at least about 33, at least about 34, at least about 35, at least about 36, at least about 37, at least about 38, at least about 39, at least about 40, at least about 41, at least about 42, at least about 43, at least about 44, at least about 45, at least about 46, at least about 47, at least about 48, at least about 49, at least 50, at least about 51, at least about 52, at least about 53, at least about 54, at least about 55, at least about 56, at least about 57, at least about 58, at least about 59, at least about 60, at least about 61, at least about 62, at least about 63, at least about 64, at least about 65, at least about 66, at least about 67, at least about 68, at least about 69, at least about 70, at least about 71, at least about 72, at least about 73, at least about 74, at least about 75, at least about 76, at least about 77, at least about 78, at least about 79, or at least about 80 basic amino acids. In some aspects, the cationic carrier moiety comprises at least 20, at least 30, at least 40, at least 50, at least 60, at least about 70, at least about 80, at least about 90, at least about 100, at least about 110, at least about 120, at least about 130, at least about 140, or at least about 150 basic amino acids.

In some aspects, the cationic carrier moiety comprises about 10 to about 60, about 15 to about 60, about 20 to about 60, about 25 to about 60, about 30 to about 60, about 35 to about 60, about 40 to about 60, about 10 to about 55, about 15 to about 55, about 20 to about 55, about 25 to about 55, about 30 to about 55, about 35 to about 55, about 40 to about 55, about 10 to about 50, about 15 to about 50, about 20 to about 50, about 25 to about 50, about 30 to about 50, about 35 to about 50, about 40 to about 50, about 10 to about 45, about 15 to about 45, about 20 to about 45, about 25 to about 45, about 30 to about 45, about 35 to about 45, about 40 to about 45, about 10 to about 40, about 15 to about 40, about 20 to about 40, about 25 to about 40, about 30 to about 40, about 35 to about 40, about 10 to about 35, about 15 to about 35, about 20 to about 35, about 25 to about 35, about 30 to about 35, about 35 to about 35, about 40 to about 35, or about 40 to about 35 basic amino acids.

In some aspects, the cationic carrier moiety comprises about 30 to about 50 basic amino acids. In some aspects, the cationic carrier moiety comprises about 10, about 20, about 30, about 40, about 50, or about 60 basic amino acids. In some aspects, the basic amino acid comprises arginine, lysine, histidine, or any combination thereof. In some aspects, the cationic carrier moiety comprises about 20, about 30, about 40, about 50, or about 60 lysines. In some aspects, the cationic carrier moity comprises about 32 lysines.

In some aspects, the crosslinking moiety comprises one or more amino acids linked to a crosslinking agent. In some aspects, the crosslinking agent comprises a thiol group, a thiol, derivative, or any combination thereof. In some aspects, the crosslinking moiety comprises a thiol group.

In some aspects, the amino acids in the crosslinking moiety comprise at least three, at least four, at least five, at least six, at least seven, at least eight, at least nine, at least ten, at least 11, at least 12, at least 13, at least 14, at last 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39 or at least 40 basic amino acids. In some aspects, the amino acids in the crosslinking moiety comprise about 1 to about 40, about 5 to about 40, about 10 to about 40, about 15 to about 40, about 20 to about 40, about 1 to about 35, about 5 to about 35, about 10 to about 35, about 15 to about 35, about 20 to about 35, about 1 to about 30, about 5 to about 30, about 10 to about 30, about 15 to about 30, about 20 to about 30, about 1 to about 25, about 5 to about 25, about 10 to about 25, about 15 to about 25, about 20 to about 25, about 1 to about 20, about 5 to about 20, about 10 to about 20, about 15 to about 20, about 1 to about 15, about 5 to about 15, about 10 to about 15, about 1 to about 10, about 5 to about 10, or about 1 to about 5 basic amino acids.

In some aspects, the amino acids in the crosslinking moiety comprise about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, or about 30 basic amino acids. In some aspects, the basic amino acids in the crosslinking moiety comprise arginine, lysine, histidine, or any combination thereof. In some aspects, the basic amino acids in the crosslinking moiety comprise about 5, about 10, about 11, about 12, about 13, about 14, about 15, about 16, about 17, about 18, about 19, about 20, about 25, about 30, or about 40 lysines. In some aspects, the basic amino acids in the crosslinking moiety comprise about 10 to about 20 lysines. In some aspects, the basic amino acids in the crosslinking moiety comprises about 16 lysines.

In some aspects, the hydrophobic moiety is capable of modulating an immune response, an inflammatory response, or a tissue microenvironment. In some aspects, the hydrophobic moiety is capable of modulating an immune response. In some aspects, the hydrophobic moiety is capable of modulating a tumor microenvironment in a subject with a tumor.

In some aspects, the hydrophobic moiety is capable of inhibiting or reducing hypoxia in the tumor microenvironment.

In some aspects, the hydrophobic moiety is capable of inhibiting or reducing an inflammatory response. In some aspects, the hydrophobic moiety is one or more amino acids linked to a nicotinic acid derivative.

In some aspects, the hydrophobic moiety comprises at least about two, at least about three, at least about four, at least about five, at least about six, at least about seven, at least about eight, at least about nine, at least about ten, at least about 11, at least about 12, at least about 13, at least about 14, at least about 15, at least about 16, at least about 17, at least about 18, at least about 19, at least about 20, at least about 21, at least about 22, at least about 23, at least about 24, at least about 25, at least about 26, at least about 27, at least about 28, at least about 29, at least about 30, at least about 31, at least about 32, at least about 33, at least about 34, at least about 35, at least about 36, at least about 37, at least about 38, at least about 39, at least about 40, at least about 41, at least about 42, at least about 43, at least about 44, at least about 45, at least about 46, at least about 47, at least about 48, at least about 49, at least about 50, at least about 51, at least about 52, at least about 53, at least about 54, at least about 55, at least about 56, at least about 57, at least about 58, at least about 59, at least about 60, at least about 61, at least about 62, at least about 63, at least about 64, at least about 65, at least about 66, at least about 67, at least about 68, at least about 69, at least about 70, at least about 71, at least about 72, at least about 73, at least about 74, at least about 75, at least about 76, at least about 77, at least about 78, at least about 79, or at least about 80 amino acids, each linked to a nicotinic acid derivative. In some aspects, the hydrophobic moiety comprises at least 20, at least 30, at least 40, at least 50, at least 60, at least about 70, at least about 80, at least about 90, at least about 100, at least about 110, at least about 120, at least about 130, at least about 140, or at least about 150 amino acids, each linked to a nicotinic acid derivative. In some aspects, the hydrophobic moiety comprises about 10 to about 60, about 15 to about 60, about 20 to about 60, about 25 to about 60, about 30 to about 60, about 35 to about 60, about 40 to about 60, about 10 to about 55, about 15 to about 55, about 20 to about 55, about 25 to about 55, about 30 to about 55, about 35 to about 55, about 40 to about 55, about 10 to about 50, about 15 to about 50, about 20 to about 50, about 25 to about 50, about 30 to about 50, about 35 to about 50, about 40 to about 50, about 10 to about 45, about 15 to about 45, about 20 to about 45, about 25 to about 45, about 30 to about 45, about 35 to about 45, about 40 to about 45, about 10 to about 40, about 15 to about 40, about 20 to about 40, about 25 to about 40, about 30 to about 40, about 35 to about 40, about 10 to about 35, about 15 to about 35, about 20 to about 35, about 25 to about 35, about 30 to about 35, about 35 to about 35, about 40 to about 35, or about 40 to about 35 amino acids, each linked to a nicotinic acid derivative. In some aspects, the hydrophobic moiety comprises about 10, about 20, about 30, about 40, about 50, or about 60 amino acids, each linked to a nicotinic acid derivative.

In some aspects, the hydrophobic moiety comprises about 10 amino acids, about 20 amino acids, about 30 amino acids, about 40 amino acids, or about 50 amino acids, each linked to a nicotinic acid derivative.

In some aspects, the cationic carrier unit comprises about 25 to about 40 lysines, the crosslinking moiety comprises about 10 to about 20 lysine-thiol, and the hydrophobic moiety comprises about 25 to about 40 lysine-nicotinic acid derivative. In some aspects, the cationic carrier moiety comprises about 30 to about 35 lysines, the crosslinking moiety comprises about 13 to about 20 lysine-thiol moieties, and the hydrophobic moiety comprises about 30 to about 35 lysine-nicotinic acid derivative moieties. In some aspects, the cationic carrier moiety comprises about 32 lysines, the crosslinking moiety comprises about 16 lysine-thiol moieties, and the hydrophobic moiety comprises about 32 lysine-nicotinic acid derivative moieties.

In some aspects, the cationic carrier unit comprises about 35 to about 60 lysines, the crosslinking moiety comprises about 5 to about 15 lysine-thiol moieties, and the hydrophobic moiety comprises about 15 to about 30 lysine-nicotinic acid derivative moieties.

In some aspects, the cationic carrier unit comprises a water-soluble biopolymer moiety, wherein the water-soluble biopolymer moiety comprises about 120 to about 130 PEG units.

In some aspects, the cationic carrier unit further comprises a targeting moiety (TM). In some aspects, the targeting moiety is capable of targeting a tissue. In some aspects, the tissue is liver, brain, kidney, lung, ovary, pancreas, thyroid, breast, stomach, or any combination thereof. In some aspects, the targeting moiety is capable of being transported by large neutral amino acid transporter 1 (LAT1). In some aspects, the targeting moiety is an amino acid. In some aspects, targeting moiety comprises a branched-chain or aromatic amino acid. In some aspects, the targeting moiety is phenylalanine, valine, leucine, and/or isoleucine. In some aspects, the amino acid is phenylalanine. In some aspects, the targeting moiety is linked to the water-soluble polymer. In some aspects, the targeting moiety is linked to the water-soluble polymer by a linker.

The present disclosure also provides a micelle comprising the cationic carrier unit disclosed herein and an anionic payload, wherein the cationic carrier moiety of the cationic carrier complex and the anionic payload are associated with each other. In some aspects, the association is a covalent bond. In other aspects, the association is a non-covalent bond. In some aspects, the association is an ionic bond.

In some aspects, the positive charge of the cationic carrier moiety of the cationic carrier unit is sufficient to form a micelle when mixed with an anionic payload in a solution, wherein the overall ionic ratio of the positive charges of the cationic carrier moiety of the cationic carrier unit and the negative charges of the anionic payload in the solution is about 1:3 and about 3:1. In some aspects, the positive charge of the cationic carrier moiety of the cationic carrier unit is sufficient to form a micelle when mixed with an anionic payload in a solution, wherein the overall ionic ratio of the positive charges of the cationic carrier moiety of the cationic carrier unit and the negative charges of the anionic payload in the solution is about 1:3, about 1:2.5; about 1:2, about 1:1.5; about 1:1, about 1:0.5; about 0.5:1, about 1.5:1; about 2:1, about 2.5:1, or about 3:1. In some aspects, the the positive charge of the cationic carrier moiety of the cationic carrier unit is sufficient to form a micelle when mixed with an anionic payload in a solution, wherein the overall ionic ratio of the positive charges of the cationic carrier moiety of the cationic carrier unit and the negative charges of the anionic payload in the solution is about 1:3, about 1:2.5; about 1:2, about 1:1.5; about 1:1, about 1:0.5; about 0.5:1, about 1.5:1; about 2:1, about 2.5:1, or about 3:1. In some aspects, the positive charge of the cationic carrier moiety of the cationic carrier unit is sufficient to form a micelle when mixed with an anionic payload in a solution, wherein the overall ionic ratio of the positive charges of the cationic carrier moiety of the cationic carrier unit and the negative charges of the anionic payload in the solution is about 1:1.

In some aspects, the cationic carrier unit is capable of protecting the anionic payload from degradation by a DNase and/or an RNase. In some aspects, the anionic payload is not conjugated to the cationic carrier unit by a covalent bond and/or the anionic payload interacts with the cationic carrier moiety of the cationic carrier unit only via an ionic interaction.

In some aspects, the half-life of the anionic payload is extended compared to the half-life of a free anionic payload not incorporated into a micelle. In some aspects, the positive charges of the cationic carrier moiety of the cationic carrier unit and the negative charges of the anionic payload in the micelle are at an ionic ratio of about 3:1, about 2.9:1, about 2.8:1, about 2.7:1, about 2.6:1, about 2.5:1, about 2.4:1, about 2.3:1, about 2.2:1, about 2:1, about 1.9:1, about 1.8:1, about 1.7:1, about 1.6:1, about 1.5:1, about 1.4:1, about 1.3:1, about 1.2:1, about 1.1:1, about 1:1, about 1:1.1, about 1:1.2, about 1:1.3, about 1:1.4, about 1:1.5, about 1:1.6, about 1:1.7, about 1:1.8, about 1:1.9, about 1:2, about 1:2.1, about 1:2.2, about 1:2.3, about 1:2.4, about 1:2.5, about 1:2.6, about 1:2.7, about 1:2.8, about 1:2.9, or about 1:3. In some aspects, the positive charges of the cationic carrier moiety of the cationic carrier unit and the negative charges of the anionic payload in the micelle are at an ionic ratio of about 3:1 to about 1:3.

In some aspects, the diameter of the micelle is between about 1 nm and 100 nm, between about 10 nm and about 100 nm, between about 10 nm and about 90 nm, between about 10 nm and about 80 nm, between about 10 nm and about 70 nm, between about 20 nm and about 100 nm, between about 20 nm and about 90 nm, between about 20 nm and about 80 nm, between about 20 nm and about 70 nm, between about 30 nm and about 100 nm, between about 30 nm and about 90 nm, between about 30 nm and about 80 nm, between about 30 nm and about 70 nm, between about 40 nm and about 100 nm, between about 40 nm and about 90 nm, between about 40 nm and about 80 nm, or between about 40 nm and about 70 nm.

In some aspects, the anionic payload comprises a nucleic acid. In some aspects, the nucleic acid comprises mRNA, miRNA, miRNA sponge, tough decoy miRNA, antimir, small RNA, rRNA, siRNA, shRNA, gDNA, cDNA, pDNA, PNA, BNA, antisense oligonucleotide (ASO), aptamer, cyclic dinucleotide, or any combination thereof. In some aspects, the nucleic acid comprises at least one nucleoside analog. In some aspects, the nucleoside analog comprises Locked Nucleic Acid (LNA); 2′-O-alkyl-RNA; 2′-amino-DNA; 2′-fluoro-DNA; arabino nucleic acid (ANA); 2′-fluoro-ANA, hexitol nucleic acid (HNA), intercalating nucleic acid (INA), constrained ethyl nucleoside (cEt), 2′-O-methyl nucleic acid (2′-OMe), 2′-O-methoxyethyl nucleic acid (2′-MOE), or any combination thereof.

In some aspects, the nucleic acid comprises a nucleotide sequence having 5 to 30 nucleotides in length. In some aspects, the nucleotide sequence is 7, 8, 9, 10, 11, 12, 13, 14, 15, 16, 17, 18, 19, 20, 21, 22, 23, 24, 25, or 26 nucleotides in length. In some aspects, the nucleotide sequence has a backbone, which comprises a phosphodiester linkage, a phosphotriester linkage, a methylphosphonate linkage, a phosphoramidate linkage, a phosphorothioate linkage, and combinations thereof. In some aspects, the nucleic acid comprises the nucleotide sequence as set forth in SEQ ID NO: 18 (miR-485 3p inhibitor).

The present disclosure also provides a composition comprising the cationic carrier unit disclosed herein and a negatively charged molecule. Also provided is a pharmaceutical composition comprising a cationic carrier unit, composition, or micelle disclosed herein, and a pharmaceutically acceptable carrier.

The present disclosure also provides a method of preparing the cationic carrier unit disclosed herein comprising linking the cationic carrier moiety to the crosslinking moiety and the hydrophobic moiety. In some aspects, the method further comprises linking a water-soluble polymer and a targeting moiety. In some aspects, the method of preparing a micelle disclosed herein comprises mixing the cationic carrier unit with the anionic payload at an ionic ratio of 1:1 in solution. In some aspects, the method of preparing a micelle disclosed herein comprises mixing the cationic carrier unit with the anionic payload at an ionic ratio of 2:1 in solution. In some aspects, the method of preparing a micelle disclosed herein comprises mixing the cationic carrier unit with the anionic payload at an ionic ratio between about 1:3 and about 3:1 in solution. In some aspects, the method further comprises purifying the micelle.

The present disclosure also provides a method of treating a disease or condition in a subject in need thereof comprising administering a micelle or the pharmaceutical composition of the present disclosure to the subject. In some aspects, the anionic payload in the core of the micelle exhibits a longer half-life than a corresponding anionic payload not integrated into a micelle. In some aspects, the subject is a mammal.

The present disclosure also provides a method to reduce inflammation in a subject suffering from a neurodegenerative disease comprising administering a therapeutically effective amount of a micelle disclosed herein to the subject.

The present disclosure also provides a method to recover and/or induce neurogenesis in a subject suffering from a neurodegenerative disease comprising administering a therapeutically effective amount of a micelle disclosed herein to the subject.

The present disclosure also provides a method to improve cognitive function in a subject suffering from a neurodegenerative disease comprising administering a therapeutically effective amount of a micelle disclosed herein to the subject.

In some aspects, the neurodegenerative disease is Alzheimer's disease.

The present disclosure also provides a method to reduce amyloid plaque burden in a subject suffering from Alzheimer's disease comprising administering a therapeutically effective amount of a micelle disclosed herein to the subject.

### BRIEF DESCRIPTION OF THE DRAWINGS

FIGS. 1A-1C show exemplary architectures of carrier units and micelles of the present disclosure. The exemplary carrier units comprise an optional tissue-specific targeting moiety, water-soluble polymer, and cationic carrier unit, comprising a cationic moiety, crosslinking moiety, and hydrophobic moiety, (which can, respectively, interact with anionic payloads) (FIG. 1A). In some aspects, the cationic carrier and anionic payload are not tethered and interact electrostatically. FIG. 1B shows a schematic diagram of an anionic payload. In some aspects, the cationic carrier and anionic payload are tethered and interact electrostatically. FIG. 1C shows a schematic diagram of a micelle comprising a cationic carrier and anionic payload of FIGS. 1A and 1B.

FIG. 2 shows an exemplary architecture of cationic carrier unit. The polymer can be modified with various hydrophobic moiety B to control the pKa characteristics of the whole micelle. R₁ to R₄ sites are potential modification site of nicotinic acid derivatives in the micelle.

FIG. 3 shows a representative scheme for nicotinic acid derivatives as a hydrophobic moiety. Electron donating group (EDG) or electron withdrawing group (EWG) can be attached to R₁ or R₂ or R₃ or R₄ site and the total pKa of the micelle can be controlled by EDG or EWG.

FIG. 4 shows the principle of pKa control of nicotinic acid derivatives by the EDG. At a different pH, the protonation of pyridine ring can be controlled by EDG.

FIG. 5 shows how hydrophobic moiety B changes with the pH. When the pH is lower than the pKa of the hydrophobic moiety, the hydrophobic moiety is protonated and increases its hydrophilicity. The changes of hydrophobicity in the hydrophobic moiety affect the micelle stability and allow the payload to be released at a low pH.

FIG. 6 shows a representative nicotinic acid scheme and its derivatives: nicotinic acid (NA), 2-methylnicotinic acid (Me-NA), and 2-methoxynicotinic acid (MeO-NA). Modification of the pyridine ring in the molecules can cause a different pKa level as shown in FIG. 3.

FIG. 7 shows exemplary pKa results of nicotinic acid (NA) and 2-methylnicotinic acid (Me-NA) measured by a titration method with KOH solution. The pKa level of NA is 5.7 and the pKa level Me-NA is 6.5 respectively.

FIGS. 8A-8C show the results of 1H-NMR measured for synthesized compound A (nicotinic acid) (FIG. 8A), compound B (2-methylnicotinic acid) (FIG. 8B), and compound C (2-methoxynicotinic acide) (FIG. 8C). In Compound A, nicotinic acid is used as a hydrophobic moiety. Compound B comprises 2-methylnicotinic acid as a hydrophobic moiety. Compound C comprises 2-methoxynicotinic acid as a hydrophobic moiety.

FIG. 9A shows Compound A, Compound B, and Compound C compositions for micelle formation with anionic payload. FIG. 9B compares the particle size distribution of the Compound A, Compound B, or Compound C micelles. Each micelle was prepared with microfluidic mixer, and the particle size of the micelles are less than 50 nm with PDI<0.2.

FIGS. 10A-10C show the results of 1H-NMR measured for synthesized compound D (nicotinic acid) (FIG. 10A), compound E (2-methylnicotinic acid) (FIG. 10B), and compound F (2-methoxynicotinic acide) (FIG. 10C). In Compound D, nicotinic acid was used as a hydrophobic moiety. Compound E comprised 2-methylnicotinic acid as a hydrophobic moiety. Compound F comprised 2-methoxynicotinic acid as a hydrophobic moiety. The pKa values were: D<E<F with reference to FIG. 6.

FIG. 11A shows Compound D, Compound E, and Compound F compositions for micelle formation with anionic payload. FIG. 11B shows the particle size distribution of micelles prepared between mRNA with either compound D, compound E, or compound F. Each micelle was prepared with a microfluidic mixer with the resulting particle size less than 60 nm and PDI <0.25.

FIG. 12 shows the time dependent comparison of endosomal formation after cellular uptake of Compound A micelle, Compound B micelle, and Compound C micelle. For endosome or lysosome observation, acridine orange staining solution was added to the cells, and Compound A, Compound B, and Compound C micelles were added in each cell. After 0, 1, 3, 5 hr incubation, time dependent color changes of the cells were observed by confocal microscopy. The orange to yellow color change shows lysosomal degradation of the cells, thereby indicating that the ASO was released from the micelles after dissociation.

### DETAILED DESCRIPTION

The present disclosure is directed to carrier units comprising a water-soluble biopolymer moiety (e.g., PEG), a charged moiety (e.g., a polylysine), a crosslinking moiety, and a hydrophobic moiety. In some aspects, the hydrophobic moiety is a nicotinic acid derivative. The cationic carrier units can be packaged into micelles when the units interact with anionic payloads, wherein the payload is located in the core of the micelle and the water-soluble biopolymer moiety is facing the solvent, wheren the crosslinking moiety crosslinks one unit to other carrior units, and wherein the hydrophobic moiety (e.g., a nicotinic acid derivative) is exposed on the surface of the micelles. Non-limiting examples of various aspects are shown in the present disclosure.

Before the present disclosure is described in greater detail, it is to be understood that this disclosure is not limited to the particular compositions or process steps described, as such can, of course, vary. As will be apparent to those of skill in the art upon reading this disclosure, each of the individual aspects described and illustrated herein has discrete components and features which can be readily separated from or combined with the features of any of the other several aspects without departing from the scope or spirit of the present disclosure. Any recited method can be carried out in the order of events recited or in any other order that is logically possible.

The headings provided herein are not limitations of the various aspects of the disclosure, which can be defined by reference to the specification as a whole. It is also to be understood that the terminology used herein is for describing particular aspects only, and is not intended to be limiting, since the scope of the present disclosure will be limited only by the appended claims.

Accordingly, the terms defined immediately below are more fully defined by reference to the specification in its entirety.

### I. Definitions

In order that the present description can be more readily understood, certain terms are first defined. Additional definitions are set forth throughout the detailed description.

It is to be noted that the term “a” or “an” entity refers to one or more of that entity; for example, “a nucleotide sequence,” is understood to represent one or more nucleotide sequences.

As such, the terms “a” (or “an”), “one or more,” and “at least one” can be used interchangeably herein. It is further noted that the claims can be drafted to exclude any optional element. As such, this statement is intended to serve as antecedent basis for use of such exclusive terminology as “solely,” “only” and the like in connection with the recitation of claim elements, or use of a negative limitation.

Furthermore, “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

It is understood that wherever aspects are described herein with the language “comprising,” otherwise analogous aspects described in terms of “consisting of” and/or “consisting essentially of” are also provided.

Unless defined otherwise, all technical and scientific terms used herein have the same meaning as commonly understood by one of ordinary skill in the art to which this disclosure is related. For example, the Concise Dictionary of Biomedicine and Molecular Biology, Juo, Pei-Show, 2nd ed., 2002, CRC Press; The Dictionary of Cell and Molecular Biology, 3rd ed., 1999, Academic Press; and the Oxford Dictionary Of Biochemistry And Molecular Biology, Revised, 2000, Oxford University Press, provide one of skill with a general dictionary of many of the terms used in this disclosure.

Units, prefixes, and symbols are denoted in their Système International de Unites (SI) accepted form. Numeric ranges are inclusive of the numbers defining the range. Where a range of values is recited, it is to be understood that each intervening integer value, and each fraction thereof, between the recited upper and lower limits of that range is also specifically disclosed, along with each subrange between such values. The upper and lower limits of any range can independently be included in or excluded from the range, and each range where either, neither or both limits are included is also encompassed within the disclosure. Thus, ranges recited herein are understood to be shorthand for all of the values within the range, inclusive of the recited endpoints. For example, a range of 1 to 10 is understood to include any number, combination of numbers, or sub-range from the group consisting of 1, 2, 3, 4, 5, 6, 7, 8, 9, and 10.

Where a value is explicitly recited, it is to be understood that values that are about the same quantity or amount as the recited value are also within the scope of the disclosure. Where a combination is disclosed, each subcombination of the elements of that combination is also specifically disclosed and is within the scope of the disclosure. Conversely, where different elements or groups of elements are individually disclosed, combinations thereof are also disclosed. Where any element of a disclosure is disclosed as having a plurality of alternatives, examples of that disclosure in which each alternative is excluded singly or in any combination with the other alternatives are also hereby disclosed; more than one element of a disclosure can have such exclusions, and all combinations of elements having such exclusions are hereby disclosed.

Nucleotides are referred to by their commonly accepted single-letter codes. Unless otherwise indicated, nucleotide sequences are written left to right in 5′ to 3′ orientation. Nucleotides are referred to herein by their commonly known one-letter symbols recommended by the IUPAC-IUB Biochemical Nomenclature Commission. Accordingly, ‘a’ represents adenine, ‘c’ represents cytosine, ‘g’ represents guanine, ‘t’ represents thymine, and ‘u’ represents uracil.

Amino acid sequences are written left to right in amino to carboxy orientation. Amino acids are referred to herein by either their commonly known three letter symbols or by the one-letter symbols recommended by the IUPAC-IUB Biochemical Nomenclature Commission.

The term “about” is used herein to mean approximately, roughly, around, or in the regions of. When the term “about” is used in conjunction with a numerical range, it modifies that range by extending the boundaries above and below the numerical values set forth. In general, the term “about” can modify a numerical value above and below the stated value by a variance of, e.g., 10 percent, up or down (higher or lower).

The terms “administration,” “administering,” and grammatical variants thereof refer to introducing a composition, such as a micelle of the present disclosure, into a subject via a pharmaceutically acceptable route. The introduction of a composition, such as a micelle of the present disclosure, into a subject is by any suitable route, including intratumorally, orally, pulmonarily, intranasally, parenterally (intravenously, intra-arterially, intramuscularly, intraperitoneally, or subcutaneously), rectally, intralymphatically, intrathecally, periocularly or topically. Administration includes self-administration and the administration by another. A suitable route of administration allows the composition or the agent to perform its intended function. For example, if a suitable route is intravenous, the composition is administered by introducing the composition or agent into a vein of the subject.

As used herein, the term “approximately,” as applied to one or more values of interest, refers to a value that is similar to a stated reference value. In certain aspects, the term “approximately” refers to a range of values that fall within 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

As used herein, the term “conserved” refers to nucleotides or amino acid residues of a polynucleotide sequence or polypeptide sequence, respectively, that are those that occur unaltered in the same position of two or more sequences being compared. Nucleotides or amino acids that are relatively conserved are those that are conserved amongst more related sequences than nucleotides or amino acids appearing elsewhere in the sequences.

Any amino acids, e.g., lysines, used in the context of the cationic carrier possess a positive charge by having its natural side group (e.g., —NH₃⁺ for lysine) or by having a modified side group. Any amino acids, e.g., lysines, used in the context of the crosslinking moiety or the hydrophobic moiety may not possess any positive charges and can be linked to a crosslinking agent (e.g., thiol) or a hydrophobic agent (e.g., nicotinic acid derivative), respectively, by an amide bond or a linker.

As used herein, the term “N/P ratio” as used herein means the molar ratio of protonated amine in a cationic carrier moiety of a cationic carrier unit to phosphate in an anionic payload when the cationic carrier unit and anionic payload are mixed together in solution.

In some aspects, two or more sequences are said to be “completely conserved” or “identical” if they are 100% identical to one another. In some aspects, two or more sequences are said to be “highly conserved” if they are at least 70% identical, at least 80% identical, at least 90% identical, or at least 95% identical to one another. In some aspects, two or more sequences are said to be “highly conserved” if they are about 70% identical, about 80% identical, about 90% identical, about 95% identical, about 98% identical, or about 99% identical to one another. In some aspects, two or more sequences are said to be “conserved” if they are at least 30% identical, at least 40% identical, at least 50% identical, at least 60% identical, at least 70% identical, at least 80% identical, at least 90% identical, or at least 95% identical to one another. In some aspects, two or more sequences are said to be “conserved” if they are about 30% identical, about 40% identical, about 50% identical, about 60% identical, about 70% identical, about 80% identical, about 90% identical, about 95% identical, about 98% identical, or about 99% identical to one another. Conservation of sequence may apply to the entire length of a polynucleotide or polypeptide or may apply to a portion, region or feature thereof.

The term “derived from,” as used herein, refers to a component that is isolated from or made using a specified molecule or organism, or information (e.g., amino acid or nucleic acid sequence) from the specified molecule or organism. For example, a nucleic acid sequence that is derived from a second nucleic acid sequence can include a nucleotide sequence that is identical or substantially similar to the nucleotide sequence of the second nucleic acid sequence. In the case of nucleotides or polypeptides, the derived species can be obtained by, for example, naturally occurring mutagenesis, artificial directed mutagenesis or artificial random mutagenesis. The mutagenesis used to derive nucleotides or polypeptides can be intentionally directed or intentionally random, or a mixture of each. The mutagenesis of a nucleotide or polypeptide to create a different nucleotide or polypeptide derived from the first can be a random event (e.g., caused by polymerase infidelity) and the identification of the derived nucleotide or polypeptide can be made by appropriate screening methods, e.g., as discussed herein. Mutagenesis of a polypeptide typically entails manipulation of the polynucleotide that encodes the polypeptide. In some aspects, a nucleotide or amino acid sequence that is derived from a second nucleotide or amino acid sequence has a sequence identity of at least about 50%, at least about 51%, at least about 52%, at least about 53%, at least about 54%, at least about 55%, at least about 56%, at least about 57%, at least about 58%, at least about 59%, at least about 60%, at least about 61%, at least about 62%, at least about 63%, at least about 64%, at least about 65%, at least about 66%, at least about 67%, at least about 68%, at least about 69%, at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% to the second nucleotide or amino acid sequence, respectively, wherein the first nucleotide or amino acid sequence retains the biological activity of the second nucleotide or amino acid sequence.

The terms “complementary” and “complementarity” refer to two or more oligomers (i.e., each comprising a nucleobase sequence), or between an oligomer and a target gene, that are related with one another by Watson-Crick base-pairing rules. For example, the nucleobase sequence “T-G-A (5′→3′),” is complementary to the nucleobase sequence “A-C-T (3′→5′).” Complementarity may be “partial,” in which less than all of the nucleobases of a given nucleobase sequence are matched to the other nucleobase sequence according to base pairing rules. For example, in some aspects, complementarity between a given nucleobase sequence and the other nucleobase sequence may be about 70%, about 75%, about 80%, about 85%, about 90% or about 95%. Or, there may be “complete” or “perfect” (100%) complementarity between a given nucleobase sequence and the other nucleobase sequence to continue the example. The degree of complementarity between nucleobase sequences has significant effects on the efficiency and strength of hybridization between the sequences.

The term “downstream” refers to a nucleotide sequence that is located 3′ to a reference nucleotide sequence. In certain aspects, downstream nucleotide sequences relate to sequences that follow the starting point of transcription. For example, the translation initiation codon of a gene is located downstream of the start site of transcription.

The terms “excipient” and “carrier” are used interchangeably and refer to an inert substance added to a pharmaceutical composition to further facilitate administration of a compound.

As used herein, the term “homology” refers to the overall relatedness between polymeric molecules, e.g. between nucleic acid molecules (e.g. DNA molecules and/or RNA molecules) and/or between polypeptide molecules. Generally, the term “homology” implies an evolutionary relationship between two molecules. Thus, two molecules that are homologous will have a common evolutionary ancestor. In the context of the present disclosure, the term homology encompasses both identity and similarity.

In some aspects, polymeric molecules are considered to be “homologous” to one another if at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 99% of the monomers in the molecule are identical (exactly the same monomer) or are similar (conservative substitutions). The term “homologous” necessarily refers to a comparison between at least two sequences (polynucleotide or polypeptide sequences).

As used herein, the term “identity” refers to the overall monomer conservation between polymeric molecules, e.g., between polypeptide molecules or polynucleotide molecules (e.g. DNA molecules and/or RNA molecules). The term “identical” without any additional qualifiers, e.g., protein A is identical to protein B, implies the sequences are 100% identical (100% sequence identity). Describing two sequences as, e.g., “70% identical,” is equivalent to describing them as having, e.g., “70% sequence identity.”

Calculation of the percent identity of two polypeptide or polynucleotide sequences, for example, can be performed by aligning the two sequences for optimal comparison purposes (e.g., gaps can be introduced in one or both of a first and a second polypeptide or polynucleotide sequences for optimal alignment and non-identical sequences can be disregarded for comparison purposes). In certain aspects, the length of a sequence aligned for comparison purposes is at least about 30%, at least about 40%, at least about 50%, at least about 60%, at least about 70%, at least about 80%, at least about 90%, at least about 95%, or about 100% of the length of the reference sequence. The amino acids at corresponding amino acid positions, or bases in the case of polynucleotides, are then compared.

When a position in the first sequence is occupied by the same amino acid as the corresponding position in the second sequence, then the molecules are identical at that position. The percent identity between the two sequences is a function of the number of identical positions shared by the sequences, taking into account the number of gaps, and the length of each gap, which needs to be introduced for optimal alignment of the two sequences. The comparison of sequences and determination of percent identity between two sequences can be accomplished using a mathematical algorithm.

Suitable software programs are available from various sources, and for alignment of both protein and nucleotide sequences. One suitable program to determine percent sequence identity is b12seq, part of the BLAST suite of program available from the U.S. government's National Center for Biotechnology Information BLAST web site (blast.ncbi.nlm.nih.gov). B12seq performs a comparison between two sequences using either the BLASTN or BLASTP algorithm. BLASTN is used to compare nucleic acid sequences, while BLASTP is used to compare amino acid sequences. Other suitable programs are, e.g., Needle, Stretcher, Water, or Matcher, part of the EMBOSS suite of bioinformatics programs and also available from the European Bioinformatics Institute (EBI) at www.ebi.ac.uk/Tools/psa.

Sequence alignments can be conducted using methods known in the art such as MAFFT, Clustal (ClustalW, Clustal X or Clustal Omega), MUSCLE, etc.

Different regions within a single polynucleotide or polypeptide target sequence that aligns with a polynucleotide or polypeptide reference sequence can each have their own percent sequence identity. It is noted that the percent sequence identity value is rounded to the nearest tenth. For example, 80.11, 80.12, 80.13, and 80.14 are rounded down to 80.1, while 80.15, 80.16, 80.17, 80.18, and 80.19 are rounded up to 80.2. It also is noted that the length value will always be an integer.

In certain aspects, the percentage identity (% ID) or of a first amino acid sequence (or nucleic acid sequence) to a second amino acid sequence (or nucleic acid sequence) is calculated as % ID=100×(Y/Z), where Y is the number of amino acid residues (or nucleobases) scored as identical matches in the alignment of the first and second sequences (as aligned by visual inspection or a particular sequence alignment program) and Z is the total number of residues in the second sequence. If the length of a first sequence is longer than the second sequence, the percent identity of the first sequence to the second sequence will be higher than the percent identity of the second sequence to the first sequence.

One skilled in the art will appreciate that the generation of a sequence alignment for the calculation of a percent sequence identity is not limited to binary sequence-sequence comparisons exclusively driven by primary sequence data. It will also be appreciated that sequence alignments can be generated by integrating sequence data with data from heterogeneous sources such as structural data (e.g., crystallographic protein structures), functional data (e.g., location of mutations), or phylogenetic data. A suitable program that integrates heterogeneous data to generate a multiple sequence alignment is T-Coffee, available at www.tcoffee.org, and alternatively available, e.g., from the EBI. It will also be appreciated that the final alignment used to calculate percent sequence identity can be curated either automatically or manually.

As used herein, the terms “isolated,” “purified,” “extracted,” and grammatical variants thereof are used interchangeably and refer to the state of a preparation of desired composition of the present disclosure, that has undergone one or more processes of purification. In some aspects, isolating or purifying as used herein is the process of removing, partially removing (e.g., a fraction) of a composition of the present disclosure from a sample containing contaminants. In some aspects, an isolated composition has no detectable undesired activity or, alternatively, the level or amount of the undesired activity is at or below an acceptable level or amount. In other aspects, an isolated composition has an amount and/or concentration of desired composition of the present disclosure, at or above an acceptable amount and/or concentration and/or activity. In other aspects, the isolated composition is enriched as compared to the starting material from which the composition is obtained. This enrichment can be by at least about 10%, at least about 20%, at least about 30%, at least about 40%, at least about 50%, at least about 60%, at least about 70%, at least about 80%, at least about 90%, at least about 95%, at least about 96%, at least about 97%, at least about 98%, at least about 99%, at least about 99.9%, at least about 99.99%, at least about 99.999%, at least about 99.9999%, or greater than 99.9999% as compared to the starting material. In some aspects, isolated preparations are substantially free of residual biological products. In some aspects, the isolated preparations are 100% free, at least about 99% free, at least about 98% free, at least about 97% free, at least about 96% free, at least about 95% free, at least about 94% free, at least about 93% free, at least about 92% free, at least about 91% free, or at least about 90% free of any contaminating biological matter. Residual biological products can include abiotic materials (including chemicals) or unwanted nucleic acids, proteins, lipids, or metabolites.

The term “linked” as used herein refers to a first amino acid sequence or polynucleotide sequence covalently or non-covalently joined to a second amino acid sequence or polynucleotide sequence, respectively. The first amino acid or polynucleotide sequence can be directly joined or juxtaposed to the second amino acid or polynucleotide sequence or alternatively an intervening sequence can covalently join the first sequence to the second sequence. The term “linked” means not only a fusion of a first polynucleotide sequence to a second polynucleotide sequence at the 5′-end or the 3′-end, but also includes insertion of the whole first polynucleotide sequence (or the second polynucleotide sequence) into any two nucleotides in the second polynucleotide sequence (or the first polynucleotide sequence, respectively). The first polynucleotide sequence can be linked to a second polynucleotide sequence by a phosphodiester bond or a linker. The linker can be, e.g., a polynucleotide.

The terms “miRNA” or “miR” or “microRNA” are used interchangeably and refer to a microRNA molecule found in eukaryotes that is involved in RNA-based gene regulation. The term will be used to refer to the single-stranded RNA molecule processed from a precursor. Names of miRNAs and their sequences related to the present disclosure are provided herein. MicroRNAs recognize and bind to target mRNAs through imperfect base pairing leading to destabilization or translational inhibition of the target mRNA and thereby downregulate target gene expression. Conversely, targeting miRNAs via molecules comprising a miRNA binding site (generally a molecule comprising a sequence complementary to the seed region of the miRNA) can reduce or inhibit the miRNA-induced translational inhibition leading to an upregulation of the target gene.

The terms “mismatch” or “mismatches” refer to one or more nucleobases (whether contiguous or separate) in an oligomer nucleobase sequence that are not matched to a target pre-mRNA according to base pairing rules. While perfect complementarity is often desired, some aspects can include one or more but preferably 6, 5, 4, 3, 2, or 1 mismatches with respect to the target pre-mRNA. Variations at any location within the oligomer are included. In certain aspects, antisense oligomers of the disclosure include variations in nucleobase sequence near the termini, variations in the interior, and if present are typically within about 6, 5, 4, 3, 2, or 1 subunits of the 5′ and/or 3′ terminus. In certain aspects, one, two, or three nucleobases can be removed and still provide on-target binding.

As used herein, the terms “modulate,” “modify,” and grammatical variants thereof, generally refer when applied to a specific concentration, level, expression, function or behavior, to the ability to alter, by increasing or decreasing, e.g., directly or indirectly promoting/stimulating/up-regulating or interfering with/inhibiting/down-regulating the specific concentration, level, expression, function or behavior, such as, e.g., to act as an antagonist or agonist. In some instances, a modulator can increase and/or decrease a certain concentration, level, activity or function relative to a control, or relative to the average level of activity that would generally be expected or relative to a control level of activity.

“Nucleic acid,” “nucleic acid molecule,” “nucleotide sequence,” “polynucleotide,” and grammatical variants thereof are used interchangeably and refer to the phosphate ester polymeric form of ribonucleosides (adenosine, guanosine, uridine or cytidine; “RNA molecules”) or deoxyribonucleosides (deoxyadenosine, deoxyguanosine, deoxythymidine, or deoxycytidine; “DNA molecules”), or any phosphoester analogs thereof, such as phosphorothioates and thioesters, in either single stranded form, or a double-stranded helix. Single stranded nucleic acid sequences refer to single-stranded DNA (ssDNA) or single-stranded RNA (ssRNA). Double stranded DNA-DNA, DNA-RNA and RNA-RNA helices are possible. The term nucleic acid molecule, and in particular DNA or RNA molecule, refers only to the primary and secondary structure of the molecule, and does not limit it to any particular tertiary forms. Thus, this term includes double-stranded DNA found, inter alia, in linear or circular DNA molecules (e.g., restriction fragments), plasmids, supercoiled DNA and chromosomes. In discussing the structure of particular double-stranded DNA molecules, sequences can be described herein according to the normal convention of giving only the sequence in the 5′ to 3′ direction along the non-transcribed strand of DNA (i.e., the strand having a sequence homologous to the mRNA). A “recombinant DNA molecule” is a DNA molecule that has undergone a molecular biological manipulation. DNA includes, but is not limited to, cDNA, genomic DNA, plasmid DNA, synthetic DNA, and semi-synthetic DNA. A “nucleic acid composition” of the disclosure comprises one or more nucleic acids as described herein.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion.

The terms “pharmaceutically-acceptable carrier,” “pharmaceutically-acceptable excipient,” and grammatical variations thereof, encompass any of the agents approved by a regulatory agency of the U.S. Federal government or listed in the U.S. Pharmacopeia for use in animals, including humans, as well as any carrier or diluent that does not cause the production of undesirable physiological effects to a degree that prohibits administration of the composition to a subject and does not abrogate the biological activity and properties of the administered compound. Included are excipients and carriers that are useful in preparing a pharmaceutical composition and are generally safe, non-toxic, and desirable.

As used herein, the term “pharmaceutical composition” refers to one or more of the compounds described herein, such as, e.g., a micelle of the present disclosure, mixed or intermingled with, or suspended in one or more other chemical components, such as pharmaceutically-acceptable carriers and excipients. One purpose of a pharmaceutical composition is to facilitate administration of preparations of micelles to a subject.

The term “polynucleotide” as used herein refers to polymers of nucleotides of any length, including ribonucleotides, deoxyribonucleotides, analogs thereof, or mixtures thereof. This term refers to the primary structure of the molecule. Thus, the term includes triple-, double- and single-stranded deoxyribonucleic acid (“DNA”), as well as triple-, double- and single-stranded ribonucleic acid (“RNA”). It also includes modified, for example by alkylation, and/or by capping, and unmodified forms of the polynucleotide.

More particularly, the term “polynucleotide” includes polydeoxyribonucleotides (containing 2-deoxy-D-ribose), polyribonucleotides (containing D-ribose), including tRNA, rRNA, hRNA, siRNA and mRNA, whether spliced or unspliced, any other type of polynucleotide which is an N- or C-glycoside of a purine or pyrimidine base, and other polymers containing normucleotidic backbones, for example, polyamide (e.g., peptide nucleic acids “PNAs”) and polymorpholino polymers, and other synthetic sequence-specific nucleic acid polymers providing that the polymers contain nucleobases in a configuration which allows for base pairing and base stacking, such as is found in DNA and RNA.

In some aspects of the present disclosure a polynucleotide can be, e.g., an oligonucleotide, such as an antisense oligonucleotide. In some aspects, the oligonucleotide is an RNA. In some aspects, the RNA is a synthetic RNA. In some aspects, the synthetic RNA comprises at least one unnatural nucleobase. In some aspects, all nucleobases of a certain class have been replaced with unnatural nucleobases (e.g., all uridines in a polynucleotide disclosed herein can be replaced with an unnatural nucleobase, e.g., 5-methoxyuridine).

The terms “polypeptide,” “peptide,” and “protein” are used interchangeably herein to refer to polymers of amino acids of any length. The polymer can comprise modified amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids such as homocysteine, ornithine, p-acetylphenylalanine, D-amino acids, and creatine), as well as other modifications known in the art. The term “polypeptide,” as used herein, refers to proteins, polypeptides, and peptides of any size, structure, or function. Polypeptides include gene products, naturally occurring polypeptides, synthetic polypeptides, homologs, orthologs, paralogs, fragments and other equivalents, variants, and analogs of the foregoing. A polypeptide can be a single polypeptide or can be a multi-molecular complex such as a dimer, trimer or tetramer. They can also comprise single chain or multichain polypeptides. Most commonly, disulfide linkages are found in multichain polypeptides. The term polypeptide can also apply to amino acid polymers in which one or more amino acid residues are an artificial chemical analogue of a corresponding naturally occurring amino acid. In some aspects, a “peptide” can be less than or equal to 50 amino acids long, e.g., about 5, 10, 15, 20, 25, 30, 35, 40, 45, or 50 amino acids long.

The terms “prevent,” “preventing,” and variants thereof as used herein, refer partially or completely delaying onset of an disease, disorder and/or condition; partially or completely delaying onset of one or more symptoms, features, or clinical manifestations of a particular disease, disorder, and/or condition; partially or completely delaying onset of one or more symptoms, features, or manifestations of a particular disease, disorder, and/or condition; partially or completely delaying progression from a particular disease, disorder and/or condition; and/or decreasing the risk of developing pathology associated with the disease, disorder, and/or condition. In some aspects, preventing an outcome is achieved through prophylactic treatment.

As used herein, “prophylactic” refers to a therapeutic or course of action used to prevent the onset of a disease or condition, or to prevent or delay a symptom associated with a disease or condition.

As used herein, a “prophylaxis” refers to a measure taken to maintain health and prevent or delay the onset of a bleeding episode, or to prevent or delay symptoms associated with a disease or condition.

As used herein, the term “similarity” refers to the overall relatedness between polymeric molecules, e.g. between polynucleotide molecules (e.g. DNA molecules and/or RNA molecules) and/or between polypeptide molecules. Calculation of percent similarity of polymeric molecules to one another can be performed in the same manner as a calculation of percent identity, except that calculation of percent similarity takes into account conservative substitutions as is understood in the art. It is understood that percentage of similarity is contingent on the comparison scale used, i.e., whether the amino acids are compared, e.g., according to their evolutionary proximity, charge, volume, flexibility, polarity, hydrophobicity, aromaticity, isoelectric point, antigenicity, or combinations thereof.

The terms “subject,” “patient,” “individual,” and “host,” and variants thereof are used interchangeably herein and refer to any mammalian subject, including without limitation, humans, domestic animals (e.g., dogs, cats and the like), farm animals (e.g., cows, sheep, pigs, horses and the like), and laboratory animals (e.g., monkey, rats, mice, rabbits, guinea pigs and the like) for whom diagnosis, treatment, or therapy is desired, particularly humans. The methods described herein are applicable to both human therapy and veterinary applications.

As used herein, the phrase “subject in need thereof” includes subjects, such as mammalian subjects, that would benefit from administration of a micelle of the disclosure, e.g., to improve hemostasis.

The phrases “systemic administration,” “administered systemically,” “peripheral administration” and “administered peripherally” as used herein mean the administration of a compound, drug or other material other than directly into the central nervous system, such that it enters the patient's system and, thus, is subject to metabolism and other like processes, for example, subcutaneous administration.

As used herein the term “therapeutically effective amount” is the amount of reagent or pharmaceutical compound comprising a micelle of the present disclosure that is sufficient to a produce a desired therapeutic effect, pharmacologic and/or physiologic effect on a subject in need thereof. A therapeutically effective amount can be a “prophylactically effective amount” as prophylaxis can be considered therapy.

The terms “treat,” “treatment,” or “treating,” as used herein refers to, e.g., the reduction in severity of a disease or condition; the reduction in the duration of a disease course; the amelioration or elimination of one or more symptoms associated with a disease or condition; the provision of beneficial effects to a subject with a disease or condition, without necessarily curing the disease or condition. The term also include prophylaxis or prevention of a disease or condition or its symptoms thereof. In one aspect, the term “treating” or “treatment” means inducing an immune response in a subject against an antigen.

The term “upstream” refers to a nucleotide sequence that is located 5′ to a reference nucleotide sequence.

### II. Carrier Units

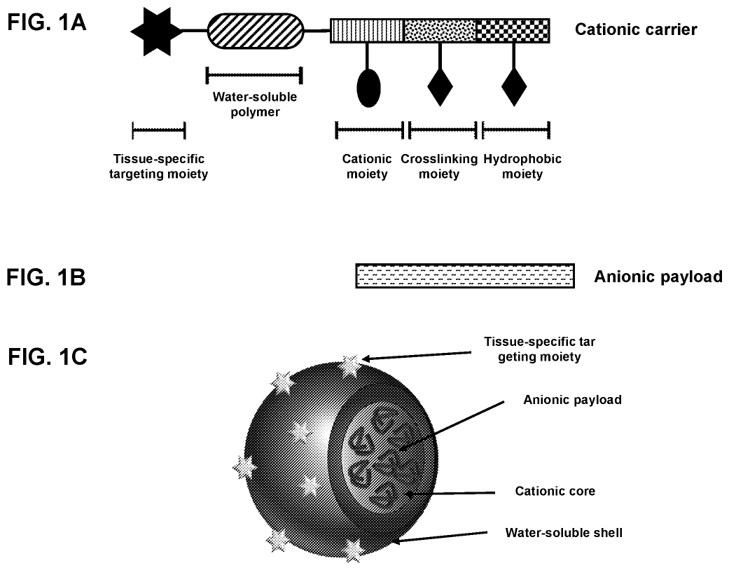

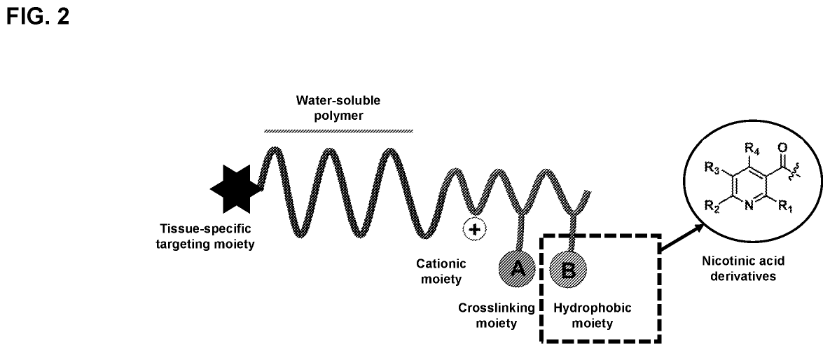

The present disclosure provides carrier units that can self-assemble into micelles or be incorporated into micelles. Carrier units of the present disclosure comprise a water-soluble biopolymer moiety (e.g., PEG), a charged carrier moiety, a crosslinking moiety, and a hydrophobic moiety. In some aspects, the charged carrier moiety is cationic (e.g., a polylysine), as exemplified in FIGS. 1 and 2. In some aspects, the cationic charged carrier moiety and an anionic payload can electrostatically interact with each other. When the cationic charged carrier moiety and the anionic charged payload are mixed together, they can neutralize each other, yielding a carrier unit: payload complex. The resulting carrier unit: payload complex can have a “head” comprising the water-soluble biopolymer moiety and a “tail” comprising the cationic carrier moiety electrostatically bound to the anionic payload.

Carrier unit: payload complexes can self-associate, alone or in combination with other molecules, to yield micelles in which the anionic payload is located in the core of the micelle and the water-soluble biopolymer moiety is facing the solvent. The term “micelles of the present disclosure” encompasses not only classic micelles but also small particles, small micelles, micelles, rod-like structures, and polymersomes. Given that polymersomes comprise a luminal space, it is to be understood that all the disclosures related to the “core” of classic micelles are equally applicable to the luminal space in polymersomes comprising carrier units of the present disclosure.

The carrier units of the present disclosure can also comprise a targeting moiety (e.g., a targeting ligand) covalently linked to the water-soluble biopolymer moiety via one or more optional linkers. Once a micelle is formed, the targeting moiety can be located on the surface of the micelle and can deliver the micelle to a specific target tissue, to a specific cell type, and/or facilitate transport across a physiological barrier (e.g., cell plasma membrane or BBB). In some aspects, the micelles of the present disclosure can comprise more than one type of targeting moiety.

The carrier units of the present disclosure can also comprise a hydrophobic moiety (HM) covalently linked to the charged cationic carrier moiety. The hydrophobic moiety can have, e.g., a therapeutic, a co-therapeutic effect, or positively affect the homeostasis of the target cell or target tissue. In some aspects, the HM comprises one or more amino acids. In some aspects, the HM compises one or more amino acids linked to a hydrophobic molecule (e.g., a nicotinic acid derivative). In some aspects, the HM comprises one or more lysine residues covalently bound to a hydrophobic molecule (e.g., a nicotinic acid derivative).

In some aspects, the anionic payload is not covalently linked to the carrier unit. However, in other aspects, the cationic payload can be covalently linked to the cationic carrier unit, e.g., a linker such as a cleavable linker.

Non-limiting examples of various aspects are shown in the present disclosure. The disclosure refers in particular to the use of cationic carrier units, e.g., to deliver anionic payloads such as nucleic acids. However, it would be apparent to a person of ordinary skill in the art that the disclosures can be equally applied to the delivery of cationic payloads or to the delivery of neutral payloads by reversing the charges of the carrier moiety and payload (i.e., using an anionic carrier moiety in the carrier unit to deliver a cationic payload), or by using a neutral payload linked to a cationic or anionic adapter that would electrostatically interact with an anionic or cationic carrier moiety, respectively.

Accordingly, in one aspect, the present disclosure provides cationic carrier units of Schemas I through Schema VI

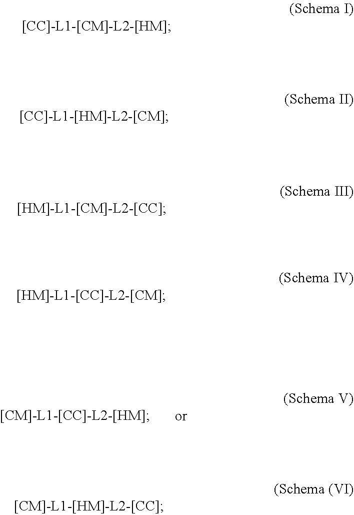

wherein
CC is a cationic carrier moiety, e.g., a polylysine;
CM is a crosslinking moiety;
HM is a hydrophobic moiety, e.g., a nicotinic acid derivative; and,
L1 and L2 are independently optional linkers, and
wherein the number of HM is less than about 50% relative to [CC] and [CM].

In some aspects, the cationic carrier unit further comprises a water-soluble polymer (WP). In some aspects, the water-soluble polymer is attached to [CC], [HM], and/or [CM]. In some aspects, the water-soluble polymer is attached to the N terminus of [CC], [HM], or [CM]. In some aspects, the water-soluble polymer is attached to the N terminus of [CC]. In some aspects, the water-soluble polymer is attached to the C terminus of [CC], [HM], or [CM]. In some aspects, the water-soluble polymer is attached to the C terminus of [CC].

In some aspects, the cationic carrier unit comprises:

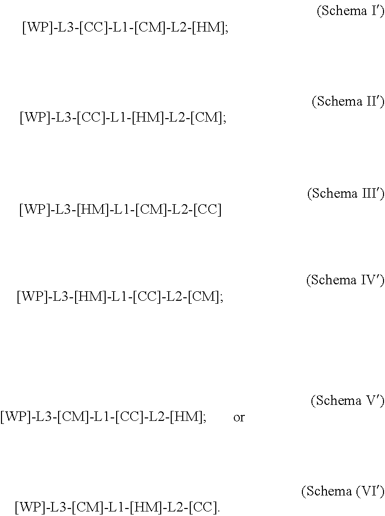

In some aspects, the cationic carrier unit is capable of interacting, e.g., electrostatically, with an anionic payload.

In some aspects of the constructs of Schema I′ to VI′ shown above, the [WP] component can be connected to at least one targeting moiety, i.e., [T]ₙ-[WP]− . . . wherein n is an integer, e.g., 1, 2 or 3.

When cationic carrier units of the present disclosure are mixed with an anionic payload (e.g., a nucleic acid) at an ionic ratio of about 5:about 1, i.e., the number of negative charges in the anionic payload is about five times higher than the number of positive charges in the cationic carrier moiety, to about 1:about 5, i.e., the number of positive charges in the cationic carrier moiety is about five times higher than the number of negative charges in the anionic payload, the neutralization of negative charges in the anionic payload by positive charges in the cationic carrier moiety mainly via electrostatic interaction leads to the formation of a cationic carrier unit: anionic payload complex having an unaltered hydrophilic portion (comprising the WP moiety) and a substantially more hydrophobic portion (resulting from the association between the cationic carrier moiety plus hydrophobic moiety and crosslinking moiety and the anionic payload).

In some aspects, the hydrophobic moiety can contribute its own positive charges to the positive charges of the cationic carrier moiety, which would interact with the negative charges of the anionic payload. It is to be understood that references to the interactions (e.g., electrostatic interactions) between a cationic carrier moiety and an anionic payload also encompass interactions between the charges of a cationic carrier moiety plus hydrophobic moiety and the charges of an anionic payload.

The increase in the hydrophobicity of the cationic carrier moiety of the cationic carrier unit due to the neutralization of its positive charges via electrostatic interaction with the negative charges of the anionic payload results in an amphipathic complex. Such amphipathic complexes can self-organize, alone or combination with other amphipathic components, into micelles. The resulting micelles comprise the WP moieties facing the solvent (i.e., the WP moieties are facing the external surface of the micelle), whereas the CC, CM, and HM moieties as well as the associated payload (e.g., a nucleotide sequence, e.g., an oligonucleotide, an siRNA, an shRNA, an “antimir,” or any combination thereof) are in the core of the micelle.

In some specific aspects, the cationic carrier unit comprises:
(a) a WP moiety, wherein the water-soluble biopolymer is a polyethylene glycol (PEG) of formula III (see below), wherein n is an integer between about 120 to about 130 (e.g., PEG is a PEG5000 or a PEG6000);
(b) a CC moiety, wherein the cationic carrier moiety comprises, e.g., about 30 to about 40 lysines (e.g., a linear poly(L-lysine)ₙ wherein n is between about 30 and about 40), a polyethylenimine (PEI), or chitosan;
(c) a CM moiety, wherein the crosslinking moiety comprises, about 10 to about 20 lysines, each of which is linked to a crosslinking agent, e.g., 10-20 lysine-thiol, and,
(c) an HM moiety, wherein the hydrophobic moiety has about 30 to about 40 lysines, each of which is linked to a nicotinic acid derivative unit.

In some aspects, the cationic carrier unit comprises:
(a) a CC moiety, wherein the cationic carrier motiey comprises about 30 to about 35 lysines, e.g., about 32 lysines;
(b) a CM moiety, wherein the crosslinking moiety comprises about 15 lysines to about 20 lysines, e.g., 16 lysines, each of which is linked to a crosslinking agent (e.g., lysine-thiol); and
(c) a HM moiety, wherein the hydrophobic moiety comprises about about 30 to about 35 lysines, e.g., about 32 lysines, each of which is linked to a hydrophobic agent (e.g., a lysine-nicotinic acid derivative).

In some specific aspects, the cationic carrier unit comprises:
(a) a CC moiety, wherein the cationic carrier motiey comprises about 25 to about 35 lysines, e.g., about 30, 31, or 32 lysines;
(b) a CM moiety, wherein the crosslinking moiety comprises about 15 lysines to about 20 lysines, e.g., 15, 16, or 17 lysines, each of which is linked to a crosslinking agent (e.g., lysine-thiol); and
(c) a HM moiety, wherein the hydrophobic moiety comprises about about 25 to about 35 lysines, e.g., about 30, 31, or 32 lysines, each of which is linked to a hydrophobic agent (e.g., a lysine-nicotinic acid derivative).

In some aspects, the cationic carrier unit comprises:
(a) a CC moiety, wherein the cationic carrier motiey comprises about 30 to about 60 lysines, e.g., about 60 lysines;
(b) a CM moiety, wherein the crosslinking moiety comprises about 5 lysines to about 15 lysines, e.g., 5 lysines, each of which is linked to a crosslinking agent (e.g., lysine-thiol); and
(c) a HM moiety, wherein the hydrophobic moiety comprises about about 15 to about 30 lysines, e.g., about 15 lysines, each of which is linked to a hydrophobic agent (e.g., a lysine-nicotinic acid derivative).

In some aspects, the cationic carrier unit comprises:
(a) a CC moiety, wherein the cationic carrier motiey comprises about 30 to about 60 lysines, e.g., about 50 lysines;
(b) a CM moiety, wherein the crosslinking moiety comprises about 5 lysines to about 15 lysines, e.g., 5 lysines, each of which is linked to a crosslinking agent (e.g., lysine-thiol); and
(c) a HM moiety, wherein the hydrophobic moiety comprises about about 15 to about 30 lysines, e.g., about 25 lysines, each of which is linked to a hydrophobic agent (e.g., a lysine-nicotinic acid derivative).

In some aspects, the cationic carrier unit comprises:
(a) a CC moiety, wherein the cationic carrier motiey comprises about 30 to about 60 lysines, e.g., about 45 lysines;
(b) a CM moiety, wherein the crosslinking moiety comprises about 5 lysines to about 15 lysines, e.g., 5 lysines, each of which is linked to a crosslinking agent (e.g., lysine-thiol); and
(c) a HM moiety, wherein the hydrophobic moiety comprises about about 15 to about 30 lysines, e.g., about 30 lysines, each of which is linked to a hydrophobic agent (e.g., a lysine-nicotinic acid derivative).

In some aspects, the cationic carrier unit comprises:
(a) a CC moiety, wherein the cationic carrier motiey comprises about 30 to about 60 lysines, e.g., about 40 lysines;
(b) a CM moiety, wherein the crosslinking moiety comprises about 5 lysines to about 15 lysines, e.g., 10 lysines, each of which is linked to a crosslinking agent (e.g., lysine-thiol); and
(c) a HM moiety, wherein the hydrophobic moiety comprises about about 15 to about 30 lysines, e.g., about 30 lysines, each of which is linked to a hydrophobic agent (e.g., a lysine-nicotinic acid derivative).

In some aspects, the cationic carrier unit comprises:
(a) a CC moiety, wherein the cationic carrier motiey comprises about 30 to about 60 lysines, e.g., about 35 lysines;
(b) a CM moiety, wherein the crosslinking moiety comprises about 5 lysines to about 15 lysines, e.g., 15 lysines, each of which is linked to a crosslinking agent (e.g., lysine-thiol); and
(c) a HM moiety, wherein the hydrophobic moiety comprises about about 15 to about 30 lysines, e.g., about 30 lysines, each of which is linked to a hydrophobic agent (e.g., lysine-nicotinic acid derivative).

In some aspects, the cationic carrier unit comprises:
(a) a CC moiety, wherein the number of the cationic carrier motiey is the number of the charge (e.g., siRNA charge)/2, e.g., about 13 to about 30 for a 14mer to 31mer;
(b) a CM moiety, wherein the number of the crosslinking moiety (e.g., lysine-thiol) is at least one, at least two, at least three, or more; and
(c) a HM moiety, wherein the hydrophobic moiety is linked to a hydrophobic agent (e.g., a lysine-nicotinic acid derivative), wherein the ratio of the CM moiety and the HM moiety is 1:3, wherein the total number of (the anionic charge/2) (X)+the number of CM (Y)+the number of HM (Z) equals or is higher than 17.

In some aspects, the cationic carrier unit further comprises at least one targeting moiety attached to the WP moiety of the cationic carrier unit. In some aspects, the number and/or density of targeting moieties displayed on the surface of the micelle can be modulated by using a specific ratio of cationic carrier units having targeting moieties to cationic carrier units not having targeting moieties. In some aspects, the ratio of cationic carrier units having a targeting moiety to cationic carrier units not having a targeting moiety is at least about 1:5, at least about 1:10, at least about 1:20, at least about 1:30, at least about 1:40, at least about 1:50, at least about 1:60, at least about 1:70, at least about 1:80, at least about 1:90, at least about 1:100, at least about 1:120, at least about 1:140, at least about 1:160, at least about 1:180, at least about 1:200, at least about 1:250, at least about 1:300, at least about 1:350, at least about 1:400, at least about 1:450, at least about 1:500, at least about 1:600, at least about 1:700, at least about 1:800, at least about 1:900, or at least about 1:1000.

In some aspects, the cationic carrier unit comprises
(i) a targeting moiety (A) which targets the transporter LAT1 (e.g., phenylalanine),
(ii) a water-soluble polymer comprising PEG,
(iii) a cationic carrier moiety comprising cationic polymer blocks which are lysine,
(iv) a crosslinking moiety comprising crosslinking polymer blocks which are lysines linked to crosslinking moieties, and
(v) a hydrophobic moiety comprising hydrophobic polymer blocks which are lysines linked to a nicotinic acid derivative.

In some aspects, the cationic carrier unit comprises
(i) a targeting moiety (A) which targets the transporter LAT1 (e.g., phenylalanine),
(ii) a water-soluble polymer which is PEG, wherein n=100-200, e.g., 100-150, e.g., 120-130,
(iii) a cationic carrier moiety comprising cationic polymer blocks, e.g., polylysine,
(iv) a crosslinking moiety comprising crosslinking polymer blocks which are lysines linked to crosslinking moieties, and
(v) a hydrophobic moiety comprising hydrophobic polymer blocks which are lysines linked to a nicotinic acid derivative.

In some aspects, the cationic carrier unit comprises
(i) a targeting moiety (A) which targets the transporter LAT1 (e.g., phenylalanine),
(ii) a water-soluble polymer which is PEG, wherein n=100-200, e.g., 100-150, e.g., 120-130,
(iii) a cationic carrier moiety comprising cationic polymer blocks, e.g., 10-100 lysines, e.g., 10-50 lysines, e.g., 30-40 lysines,
(iv) a crosslinking moiety comprising crosslinking polymer blocks which are lysines linked to crosslinking moieties, and
(v) a hydrophobic moiety comprising hydrophobic polymer blocks which are lysines linked to a nicotinic acid derivative.

In some aspects, the cationic carrier unit comprises
(i) a targeting moiety (A) which targets the transporter LAT1 (e.g., phenylalanine),
(ii) a water-soluble polymer which is PEG, wherein n=100-200, e.g., 100-150, e.g., 120-130,
(iii) a cationic carrier moiety comprising cationic polymer blocks, e.g., 10-100 lysines, e.g., 10-50 lysines, e.g., 30-40 lysines,
(iv) a crosslinking moiety comprising crosslinking polymer blocks which are lysines linked to crosslinking moieties, e.g., 10-30 lysines-thiol, e.g., 10-20 lysines-thiol, and
(v) a hydrophobic moiety comprising hydrophobic polymer blocks which are lysines linked to a nicotinic acid derivative.

In some aspects, the cationic carrier unit comprises
(i) a targeting moiety (A) which targets the transporter LAT1 (e.g., phenylalanine),
(ii) a water-soluble polymer which is PEG, wherein n=100-200, e.g., 100-150, e.g., 120-130,
(iii) a cationic carrier moiety comprising cationic polymer blocks, e.g., 10-100 lysines, e.g., 10-50 lysines, e.g., 30-40 lysines,
(iv) a crosslinking moiety comprising crosslinking polymer blocks which are lysines linked to crosslinking moieties, e.g., 10-30 lysines-thiol, e.g., 10-20 lysines-thiol, and
(v) a hydrophobic moiety comprising hydrophobic polymer blocks which are lysines linked to nicotinic acid derivative, e.g., 10-50 lysines-nicotinic acid derivative, e.g., 30-40 lysines-nicotinic acid derivative.

In some aspects, the cationic carrier unit comprises a HM, wherein the number of HM is between about 50% and about 1%, between about 50% and about 5%, between about 50% and about 10%, between about 50% and about 15%, between about 50% and about 20%, between about 50% and about 25%, between about 50% and about 30%, between about 50% and about 35%, between about 50% and about 40%, between about 50% and about 45%, between about 45% and about 1%, between about 45% and about 5%, between about 45% and about 10%, between about 45% and about 15%, between about 45% and about 20%, between about 45% and about 25%, between about 45% and about 30%, between about 45% and about 35%, between about 45% and about 40%, between about 40% and 1%, between about 50% and about 5%, between about 40% and about 10%, between about 40% and about 15%, between about 40% and about 20%, between about 40% and about 25%, between about 40% and about 30%, between about 40% and about 35%, between about 35% and about 1%, between about 35% and about 5%, between about 35% and about 10%, between about 35% and about 15%, between about 35% and about 20%, between about 35% and about 25%, between about 35% and about 30%, between about 30% and about 1%, between about 30% and about 5%, between about 30% and about 10%, between about 30% and about 15%, between about 30 and about 20%, between about 30% and about 25%, between about 25% and about 1%, between about 25% and about 5%, between about 25% and about 10%, between about 25% and about 15%, between about 25% and about 20%, between about 20% and about 1%, between about 20% and about 5%, between about 20% and about 10%, between about 20% and about 15%, between about 15% and about 1%, between about 15% and about 5%, between about 15% and about 10%, between about 10% to about 1%, between about 10% to about 5%, or between about 5% to about 1% relative to [CC] and [CM]. In some aspects, the number of HM is between about 50% and about 40%, between about 40% and about 30%, between about 30% and about 20%, between about 20% and about 10%, between about 10% and about 5%, or between about 5% and about 1%. In some aspects, the number of HM is about 50%, about 45%, about 40%, about 35%, about 30%, about 25%, about 20%, about 15%, about 10%, about 5%, or about 1%. In some aspects, the number of HM is about 40% relative to [CC] and [CM]. In some aspects, the number of HM is expressed as the percentage of [HM] relative to [CC] and [CM].

In some aspects, the cationic carrier unit of the present disclosure interacts with an antisense oligonucleotide payload targeting miR-485-3p, e.g., AGAGAGGAGAGCCGUGUAUGAC (SEQ ID NO: 18). In some aspects, the carrier unit complexed with the payload forms a micelle.

In some aspects, the nicotinic acid derivative units are introduced into the side chains of the HM moiety, e.g., by a coupling reaction between —NH₂ groups in the lysines and —COOH groups of the nicotinic acid derivative, in the presence of suitable conjugation reagents, for example, 1-ethyl-3-(3-dimethylaminopropyl)-carbodiimide (EDC) and N-hydroxy succinimide (NHS).

The present disclosure provides a composition comprising a carrier unit (e.g., a cationic carrier unit) of the present disclosure. In other aspects, the present disclosure provides complexes comprising a carrier unit (e.g., a cationic carrier unit unit) of the present disclosure non-covalently attached to a payload (e.g., an anionic payload, such a nucleotide sequence, e.g., an oligonucleotide, an siRNA, an shRNA, an “antimir,” or any combination thereof), wherein the carrier unit and the payload interact electrostatically. In other aspects, the present disclosure provides conjugates comprising a carrier unit (e.g., a cationic carrier unit unit) of the present disclosure covalently attached to a payload (e.g., an anionic payload such a nucleotide sequence, e.g., an oligonucleotide, an siRNA, an shRNA, an “antimir,” or any combination thereof), wherein the carrier unit and the payload interact electrostatically. In some aspects, the carrier unit and the payload can be linked via a cleavable linker. In some aspects, the carrier unit and the payload, in addition to interacting electrostatically, can interact covalently (e.g., after electrostatic interaction the carrier unit and the payload can be “locked” via a disulfide bond or a cleavable bond).

In some specific aspects, the cationic carrier unit comprises a water-soluble polymer comprising a PEG with about 120 to about 130 units, a cationic carrier moiety comprising a polylysine with about 30 to about 40 lysine units, a crosslinking moiety comprising about 10 to about 20 lysines-thiol units, and a hydrophobic moiety comprising about 30 to about 40 lysines linked to nicotinic acid derivative units.

In some aspects, the cationic carrier unit is associated with a negatively charged payload (e.g., a nucleotide sequence, e.g., an oligonucleotide (e.g., an antisense oligonucleotide), an siRNA, an shRNA, an “antimir,” or any combination thereof), which interacts with the cationic carrier unit via at least one ionic bond (i.e., via electrostatic interaction) with the cationic carrier moiety of the cationic carrier unit.

The specific components of the cationic carrier units of the present disclosure are disclosed in detail below.

a. Water-Soluble Biopolymer

In some aspects, the cationic carrier units of the present disclosure comprise at least one water-soluble biopolymer. The term “water-soluble biopolymer” as used herein refers to a biocompatible, biologically inert, non-immunogenic, non-toxic, and hydrophilic polymer, e.g., PEG.

In some aspects, the water-soluble polymer comprises poly(alkylene glycols), poly(oxyethylated polyol), poly(olefinic alcohol), poly(vinylpyrrolidone), poly(hydroxyalkylmethacrylamide), poly(hydroxyalkylmethacrylate), poly(saccharides), poly(a-hydroxy acid), poly(vinyl alcohol), polyglycerol, polyphosphazene, polyoxazolines (“POZ”) poly(N-acryloylmorpholine), or any combinations thereof. In some aspects, the water-soluble biopolymer is linear, branched, or dendritic.

In some aspects, the water-soluble biopolymer comprises polyethylene glycol (“PEG”), polyglycerol (“PG”), or poly(propylene glycol) (“PPG”). PPG is less toxic than PEG, so many biological products are now produced in PPG instead of PEG.

In some aspects, the water-soluble biopolymer comprises a PEG characterized by a formula R³—(O—CH₂—CH₂)ₙ— or R₃—(O—CH₂—CH₂)ₙ—O— with R³ being hydrogen, methyl or ethyl and n having a value from 2 to 200. In some aspects, the PEG has the formula

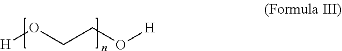

wherein n is an integer of 1 to 1000.

In some aspects, the n of the PEG has a value of 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 189, 181, 182, 183, 184, 185, 186, 187, 188, 189, 190, 191, 192, 193, 194, 195, 196, 197, 198, 199, or 200.

In some aspects, n is at least about 10, at least about 20, at least about 30, at least about 40, at least about 50, at least about 60, at least about 70, at least about 80, at least about 90, at least about 100, at least about 110, at least 120, at least about 130, at least about 140, at least about 150, at least about 160, at least about 170, at least about 180, at least about 190, at least about 200, at least about 210, at least about 220, at least about 230, at least about 240, at least about 250, at least about 260, at least about 270, at least about 280, at least about 290, at least about 300, at least about 310, at least about 320, at least about 330, at least about 340, at least about 350, at least about 360, at least about 370, at least about 380, at least about 390, at least about 400, at least about 410, at least about 420, at least about 430, at least about 440, at least about 450, at least about 460, at least about 470, at least about 480, at least about 490, at least about 500, at least about 510, at least about 520, at least about 530, at least about 540, at least about 550, at least about 560, at least about 670, at least about 580, at least about 590, at least about 600, at least about 610, at least about 620, at least about 630, at least about 640, at least about 650, at least about 660, at least about 670, at least about 680, at least about 690, at least about 700, at least about 710, at least about 720, at least about 730, at least about 740, at least about 750, at least about 760, at least about 770, at least about 780, at least about 790, at least about 800, at least about 810, at least about 820, at least about 830, at least about 840, at least about 850, at least about 860, at least about 870, at least about 880, at least about 890, at least about 900, at least about 910, at least about 920, at least about 930, at least about 940, at least about 950, at least about 960, at least about 970, at least about 980, at least about 990, or about 1000.

In some aspects, n is between about 50 and about 100, between about 100 and about 150, between about 150 and about 200, between about 200 and about 250, between about 250 and about 300, between about 300 and about 350, between about 350 and about 400, between about 400 and about 450, between about 450 and about 500, between about 500 and about 550, between about 550 and about 600, between about 600 and about 650, between about 650 and about 700, between about 700 and about 750, between about 750 and about 800, between about 800 and about 850, between about 850 and about 900, between about 900 and about 950, or between about 950 and about 1000.

In some aspects, n is at least about 80, at least about 81, at least about 82, at least about 83, at least about 84, at least about 85, at least about 86, at least about 87, at least about 88, at least about 89, at least about 90, at least about 91, at least about 92, at least about 93, at least about 94, at least about 95, at least about 96, at least about 97, at least about 98, at least about 99, at least about 100, at least about 101, at least about 102, at least about 103, at least about 104, at least about 105, at least about 106, at least about 107, at least about 108, at least about 109, at least 110, at least about 111, at least about 112, at least about 113, at least about 114, at least about 115, at least about 116, at least about 117, at least about 118, at least about 119, at least about 120, at least about 121, at least about 122, at least about 123, at least about 124, at least about 125, at least about 126, at least about 127, at least about 128, at least about 129, at least about 130, at least about 131, at least about 132, at least about 133, at least about 134, at least about 135, at least about 136, at least about 137, at least about 138, at least about 139, at least about 140, at least about 141, at least about 142, at least about 143, at least about 144, at least about 145, at least about 146, at least about 147, at least about 148, at least about 149, at least about 150, at least about 151, at least about 152, at least about 153, at least about 154, at least about 155, at least about 156, at least about 157, at least about 158, at least about 159, or at least about 160.

In some aspects, n is about 80 to about 90, about 90 to about 100, about 100 to about 110, about 110 to about 120, about 120 to about 130, about 130 to about 140, about 140 to about 150, about 150 to about 160, about 85 to about 95, about 95 to about 105, about 105 to about 115, about 115 to about 125, about 125 to about 135, about 135 to about 145, about 145 to about 155, about 155 to about 165, about 80 to about 100, about 100 to about 120, about 120 to about 140, about 140 to about 160, about 85 to about 105, about 105 to about 125, about 125 to about 145, or about 145 to about 165.

In some aspects, n is about 100 to about 150. In some aspects, n is about 100 to about 140. In some aspects, n is about 100 to about 130. In some aspects, n is about 110 to about 150. In some aspects, n is about 110 to about 140. In some aspects, n is about 110 to about 130. In some aspects, n is about 110 to about 120. In some aspects, n is about 120 to about 150. In some aspects, n is about 120 to about 140. In some aspects, n is about 120 to about 130. In some aspects, n is about 130 to about 150. In some aspects, n is about 130 to about 140.

Thus, is some aspects, the PEG is a branched PEG. Branched PEGs have three to ten PEG chains emanating from a central core group. In certain aspects, the PEG moiety is a monodisperse polyethylene glycol. In the context of the present disclosure, a monodisperse polyethylene glycol (mdPEG) is a PEG that has a single, defined chain length and molecular weight. mdPEGs are typically generated by separation from the polymerization mixture by chromatography. In certain formulae, a monodisperse PEG moiety is assigned the abbreviation mdPEG.

In some aspects, the PEG is a Star PEG. Star PEGs have 10 to 100 PEG chains emanating from a central core group. In some aspects, the PEG is a Comb PEG. Comb PEGs have multiple PEG chains normally grafted onto a polymer backbone.

In certain aspects, the PEG has a molar mass (e.g., a number average molecular weight (Mn)) between about 1000 g/mol and about 2000 g/mol, between about 2000 g/mol and about 3000 g/mol, between about 3000 g/mol to about 4000 g/mol, between about 4000 g/mol and about 5000 g/mol, between about 5000 g/mol and about 6000 g/mol, between about 6000 g/mol and about 7000 g/mol, or between 7000 g/mol and about 8000 g/mol.

In some aspects, the PEG is PEG₁₀₀, PEG₂₀₀, PEG₃₀₀, PEG₄₀₀, PEG₅₀₀, PEG₆₀₀, PEG₇₀₀, PEG₈₀₀, PEG₉₀₀, PEG₁₀₀₀, PEG₁₁₀₀, PEG₁₂₀₀, PEG₁₃₀₀, PEG₁₄₀₀, PEG₁₅₀₀, PEG₁₆₀₀, PEG₁₇₀₀, PEG₁₈₀₀, PEG₁₉₀₀, PEG₂₀₀₀, PEG₂₁₀₀, PEG₂₂₀₀, PEG₂₃₀₀, PEG₂₄₀₀, PEG₂₅₀₀, PEG₁₆₀₀, PEG₁₇₀₀, PEG₁₈₀₀, PEG₁₉₀₀, PEG₂₀₀₀, PEG₂₁₀₀, PEG₂₂₀₀, PEG₂₃₀₀, PEG₂₄₀₀, PEG₂₅₀₀, PEG₂₆₀₀, PEG₂₇₀₀, PEG₂₈₀₀, PEG₂₉₀₀, PEG₃₀₀₀, PEG₃₁₀₀, PEG₃₂₀₀, PEG₃₃₀₀, PEG₃₄₀₀, PEG₃₅₀₀, PEG₃₆₀₀, PEG₃₇₀₀, PEG₃₈₀₀, PEG₃₉₀₀, PEG₄₀₀₀, PEG₄₁₀₀, PEG₄₂₀₀, PEG₄₃₀₀, PEG₄₄₀₀, PEG₄₅₀₀, PEG₄₆₀₀, PEG₄₇₀₀, PEG₄₈₀₀, PEG₄₉₀₀, PEG₅₀₀₀, PEG₅₁₀₀, PEG₅₂₀₀, PEG₅₃₀₀, PEG₅₄₀₀, PEG₅₅₀₀, PEG₅₆₀₀, PEG₅₇₀₀, PEG₅₈₀₀, PEG₅₉₀₀, PEG₆₀₀₀, PEG₆₁₀₀, PEG₆₂₀₀, PEG₆₃₀₀, PEG₆₄₀₀, PEG₆₅₀₀, PEG₆₆₀₀, PEG₆₇₀₀, PEG₆₈₀₀, PEG₆₉₀₀, PEG₇₀₀₀, PEG₇₁₀₀, PEG₇₂₀₀, PEG₇₃₀₀, PEG₇₄₀₀, PEG₇₅₀₀, PEG₇₆₀₀, PEG₇₇₀₀, PEG₇₈₀₀, PEG₇₉₀₀, or PEG₈₀₀₀. In some aspects, the PEG is PEG₅₀₀₀. In some aspects, the PEG is PEG₆₀₀₀. In some aspects, the PEG is PEG₄₀₀₀.

In some aspects, the PEG is monodisperse, e.g., mPEG₁₀₀, MPEG₂₀₀, mPEG₃₀₀, MPEG₄₀₀, mPEG₅₀₀, mPEG₆₀₀, MPEG₇₀₀, MPEG₈₀₀, MPEG₉₀₀, MPEG₁₀₀₀, MPEG₁₁₀₀, mPEG₁₂₀₀, MPEG₁₃₀₀, MPEG₁₄₀₀, MPEG₁₅₀₀, MPEG₁₆₀₀, MPEG₁₇₀₀, MPEG₁₈₀₀, MPEG₁₉₀₀, mPEG₂₀₀₀, MPEG₂₁₀₀, MPEG₂₂₀₀, MPEG₂₃₀₀, MPEG₂₄₀₀, MPEG₂₅₀₀, MPEG₁₆₀₀, mPEG₁₇₀₀, mPEG₁₈₀₀, mPEG₁₉₀₀, MPEG₂₀₀₀, MPEG₂₁₀₀, MPEG₂₂₀₀, MPEG₂₃₀₀, MPEG₂₄₀₀, MPEG₂₅₀₀, mPEG₂₆₀₀, MPEG₂₇₀₀, MPEG₂₈₀₀, MPEG₂₉₀₀, MPEG₃₀₀₀, MPEG₃₁₀₀, mPEG₃₂₀₀, mPEG₃₃₀₀, mPEG₃₄₀₀, mPEG₃₅₀₀, mPEG₃₆₀₀, mPEG₃₇₀₀, mPEG₃₈₀₀, mPEG₃₉₀₀, MPEG₄₀₀₀, MPEG₄₁₀₀, mPEG₄₂₀₀, MPEG₄₃₀₀, MPEG₄₄₀₀, MPEG₄₅₀₀, MPEG₄₆₀₀, MPEG₄₇₀₀, mPEG₄₈₀₀, MPEG₄₉₀₀, mPEG₅₀₀₀, mPEG₅₁₀₀, mPEG₅₂₀₀, mPEG₅₃₀₀, MPEG₅₄₀₀, mPEG₅₅₀₀, MPEG₅₆₀₀, MPEG₅₇₀₀, mPEG₅₈₀₀, mPEG₅₉₀₀, mPEG₆₀₀₀, mPEG₆₁₀₀, MPEG₆₂₀₀, MPEG₆₃₀₀, MPEG₆₄₀₀, MPEG₆₅₀₀, mPEG₆₆₀₀, MPEG₆₇₀₀, mPEG₆₈₀₀, mPEG₆₉₀₀, mPEG₇₀₀₀, mPEG₇₁₀₀, mPEG₇₂₀₀, MPEG₇₃₀₀, mPEG₇₄₀₀, mPEG₇₅₀₀, mPEG₇₆₀₀, mPEG₇₇₀₀, m PEG₇₈₀₀, MPEG₇₉₀₀, or mPEG₈₀₀₀. In some aspects, the mPEG is mPEG₅₀₀₀. In some aspects, the mPEG is mPEG₆₀₀₀. In some aspects, the mPEG is mPEG₄₀₀₀.

In some aspects, the water-soluble biopolymer moiety is a polyglycerol (PG) described by the formula ((R₃—O—(CH₂—CHOH—CH₂O)ₙ—) with R₃ being hydrogen, methyl or ethyl, and n is an integer having a value from 3 to 200. In some aspects, the water-soluble biopolymer moiety is a branched polyglycerol described by the formula (R³—O—(CH₂—CHOR⁵—CH₂—O)ₙ—) with R⁵ being hydrogen or a linear glycerol chain described by the formula (R³—O—(CH₂—CHOH—CH₂—O)ₙ—) and R³ being hydrogen, methyl or ethyl. In some aspects, the water-soluble biopolymer moiety is a hyperbranched polyglycerol described by the formula (R³—O—(CH₂—CHOR⁵—CH₂—O)ₙ—) with R⁵being hydrogen or a glycerol chain described by the formula (R³—O—(CH₂—CHOR⁶—CH₂—O)ₙ—), with R⁶ being hydrogen or a glycerol chain of the formula (R₃—O—(CH₂—CHOR⁷—CH₂—O)ₙ—), with R⁷ being hydrogen or a linear glycerol chain described by the formula (R³—O—(CH₂—CHOH—CH₂—O)ₙ—) and R³ being hydrogen, methyl or ethyl. Hyperbranched glycerol and methods for its synthesis are described in Oudshorn et al. (2006) Biomaterials 27:5471-5479; Wilms et al. (2010) Acc. Chem. Res. 43, 129-41, and references cited therein.

In certain aspects, the PG has a molar mass (e.g., a number average molecular weight (Mn)) between about 1000 g/mol and about 2000 g/mol, between about 2000 g/mol and about 3000 g/mol, between about 3000 g/mol to about 4000 g/mol, between about 4000 g/mol and about 5000 g/mol, between about 5000 g/mol and about 6000 g/mol, between about 6000 g/mol and about 7000 g/mol, or between 7000 g/mol and about 8000 g/mol.

In some aspects, the PG is PG₁₀₀, PG₂₀₀, PG₃₀₀, PG₄₀₀, PG₅₀₀, PG₆₀₀, PG₇₀₀, PG₈₀₀, PG₉₀₀, PG₁₀₀₀, PG₁₁₀₀, PG₁₂₀₀, PG₁₃₀₀, PG₁₄₀₀, PG₁₅₀₀, PG₁₆₀₀, PG₁₇₀₀, PG₁₈₀₀, PG₁₉₀₀, PG₂₀₀₀, PG₂₁₀₀, PG₂₂₀₀, PG₂₃₀₀, PG₂₄₀₀, PG₂₅₀₀, PG₁₆₀₀, PG₁₇₀₀, PG₁₈₀₀, PG₁₉₀₀, PG₂₀₀₀, PG₂₁₀₀, PG₂₂₀₀, PG₂₃₀₀, PG₂₄₀₀, PG₂₅₀₀, PG₂₆₀₀, PG₂₇₀₀, PG₂₈₀₀, PG₂₉₀₀, PG₃₀₀₀, PG₃₁₀₀, PG₃₂₀₀, PG₃₃₀₀, PG₃₄₀₀, PG₃₅₀₀, PG₃₆₀₀, PG₃₇₀₀, PG₃₈₀₀, PG₃₉₀₀, PG₄₀₀₀, PG₄₁₀₀, PG₄₂₀₀, PG₄₃₀₀, PG₄₄₀₀, PG₄₅₀₀, PG₄₆₀₀, PG₄₇₀₀, PG₄₈₀₀, PG₄₉₀₀, PG₅₀₀₀, PG₅₁₀₀, PG₅₂₀₀, PG₅₃₀₀, PG₅₄₀₀, PG₅₅₀₀, PG₅₆₀₀, PG₅₇₀₀, PG₅₈₀₀, PG₅₉₀₀, PG₆₀₀₀, PG₆₁₀₀, PG₆₂₀₀, PG₆₃₀₀, PG₆₄₀₀, PG₆₅₀₀, PG₆₆₀₀, PG₆₇₀₀, PG₆₈₀₀, PG₆₉₀₀, PG₇₀₀₀, PG₇₁₀₀, PG₇₂₀₀, PG₇₃₀₀, PG₇₄₀₀, PG₇₅₀₀, PG₇₆₀₀, PG₇₇₀₀, PG₇₈₀₀, PG₇₉₀₀, or PG₈₀₀₀. In some aspects, the PG is PG₅₀₀₀. In some aspects, the PG is PG₆₀₀₀. In some aspects, the PG is PG₄₀₀₀.

In some aspects, the PG is monodisperse, e.g., mPG₁₀₀, mPG₂₀₀, mPG₃₀₀, mPG₄₀₀, mPG₅₀₀, mPG₆₀₀, mPG₇₀₀, mPG₈₀₀, mPG₉₀₀, mPG₁₀₀₀, mPG₁₁₀₀, mPG₁₂₀₀, mPG₁₃₀₀, mPG₁₄₀₀, mPG₁₅₀₀, mPG₁₆₀₀, mPG₁₇₀₀, mPG₁₈₀₀, mPG₁₉₀₀, mPG₂₀₀₀, mPG₂₁₀₀, mPG₂₂₀₀, mPG₂₃₀₀, mPG₂₄₀₀, mPG₂₅₀₀, mPG₁₆₀₀, mPG₁₇₀₀, mPG₁₈₀₀, mPG₁₉₀₀, mPG₂₀₀₀, mPG₂₁₀₀, mPG₂₂₀₀, mPG₂₃₀₀, mPG₂₄₀₀, mPG₂₅₀₀, mPG₂₆₀₀, mPG₂₇₀₀, mPG₂₈₀₀, mPG₂₉₀₀, mPG₃₀₀₀, mPG₃₁₀₀, mPG₃₂₀₀, mPG₃₃₀₀, mPG₃₄₀₀, mPG₃₅₀₀, mPG₃₆₀₀, mPG₃₇₀₀, mPG₃₈₀₀, mPG₃₉₀₀, mPG₄₀₀₀, mPG₄₁₀₀, mPG₄₂₀₀, mPG₄₃₀₀, mPG₄₄₀₀, mPG₄₅₀₀, mPG₄₆₀₀, mPG₄₇₀₀, mPG₄₈₀₀, mPG₄₉₀₀, mPG₅₀₀₀, mPG₅₁₀₀, mPG₅₂₀₀, mPG₅₃₀₀, mPG₅₄₀₀, mPG₅₅₀₀, mPG₅₆₀₀, mPG₅₇₀₀, mPG₅₈₀₀, mPG₅₉₀₀, mPG₆₀₀₀, mPG₆₁₀₀, mPG₆₂₀₀, mPG₆₃₀₀, mPG₆₄₀₀, mPG₆₅₀₀, mPG₆₆₀₀, mPG₆₇₀₀, mPG₆₈₀₀, mPG₆₉₀₀, mPG₇₀₀₀, mPG₇₁₀₀, mPG₇₂₀₀, mPG₇₃₀₀, mPG₇₄₀₀, mPG₇₅₀₀, mPG₇₆₀₀, mPG₇₇₀₀, m PG₇₈₀₀, mPG₇₉₀₀, or mPG₈₀₀₀.

In some aspects, the water-soluble biopolymer comprises poly(propylene glycol) (“PPG”). In some aspects, PPG is characterized by the following formula, with n being an integer having a value from 1 to 1000.

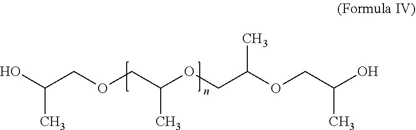

In some aspects, the n of the PPG has a value of 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 189, 181, 182, 183, 184, 185, 186, 187, 188, 189, 190, 191, 192, 193, 194, 195, 196, 197, 198, 199, or 200.

In some aspects, n of the PPG is at least about 10, at least about 20, at least about 30, at least about 40, at least about 50, at least about 60, at least about 70, at least about 80, at least about 90, at least about 100, at least about 110, at least 120, at least about 130, at least about 140, at least about 150, at least about 160, at least about 170, at least about 180, at least about 190, at least about 200, at least about 210, at least about 220, at least about 230, at least about 240, at least about 250, at least about 260, at least about 270, at least about 280, at least about 290, at least about 300, at least about 310, at least about 320, at least about 330, at least about 340, at least about 350, at least about 360, at least about 370, at least about 380, at least about 390, at least about 400, at least about 410, at least about 420, at least about 430, at least about 440, at least about 450, at least about 460, at least about 470, at least about 480, at least about 490, at least about 500, at least about 510, at least about 520, at least about 530, at least about 540, at least about 550, at least about 560, at least about 670, at least about 580, at least about 590, at least about 600, at least about 610, at least about 620, at least about 630, at least about 640, at least about 650, at least about 660, at least about 670, at least about 680, at least about 690, at least about 700, at least about 710, at least about 720, at least about 730, at least about 740, at least about 750, at least about 760, at least about 770, at least about 780, at least about 790, at least about 800, at least about 810, at least about 820, at least about 830, at least about 840, at least about 850, at least about 860, at least about 870, at least about 880, at least about 890, at least about 900, at least about 910, at least about 920, at least about 930, at least about 940, at least about 950, at least about 960, at least about 970, at least about 980, at least about 990, or about 1000.

In some aspects, the n of the PPG is between about 50 and about 100, between about 100 and about 150, between about 150 and about 200, between about 200 and about 250, between about 250 and about 300, between about 300 and about 350, between about 350 and about 400, between about 400 and about 450, between about 450 and about 500, between about 500 and about 550, between about 550 and about 600, between about 600 and about 650, between about 650 and about 700, between about 700 and about 750, between about 750 and about 800, between about 800 and about 850, between about 850 and about 900, between about 900 and about 950, or between about 950 and about 1000.

In some aspects, the n of the PPG is at least about 80, at least about 81, at least about 82, at least about 83, at least about 84, at least about 85, at least about 86, at least about 87, at least about 88, at least about 89, at least about 90, at least about 91, at least about 92, at least about 93, at least about 94, at least about 95, at least about 96, at least about 97, at least about 98, at least about 99, at least about 100, at least about 101, at least about 102, at least about 103, at least about 104, at least about 105, at least about 106, at least about 107, at least about 108, at least about 109, at least 110, at least about 111, at least about 112, at least about 113, at least about 114, at least about 115, at least about 116, at least about 117, at least about 118, at least about 119, at least about 120, at least about 121, at least about 122, at least about 123, at least about 124, at least about 125, at least about 126, at least about 127, at least about 128, at least about 129, at least about 130, at least about 131, at least about 132, at least about 133, at least about 134, at least about 135, at least about 136, at least about 137, at least about 138, at least about 139, at least about 140, at least about 141, at least about 142, at least about 143, at least about 144, at least about 145, at least about 146, at least about 147, at least about 148, at least about 149, at least about 150, at least about 151, at least about 152, at least about 153, at least about 154, at least about 155, at least about 156, at least about 157, at least about 158, at least about 159, or at least about 160.

In some aspects, the n of the PPG is about 80 to about 90, about 90 to about 100, about 100 to about 110, about 110 to about 120, about 120 to about 130, about 130 to about 140, about 140 to about 150, about 150 to about 160, about 85 to about 95, about 95 to about 105, about 105 to about 115, about 115 to about 125, about 125 to about 135, about 135 to about 145, about 145 to about 155, about 155 to about 165, about 80 to about 100, about 100 to about 120, about 120 to about 140, about 140 to about 160, about 85 to about 105, about 105 to about 125, about 125 to about 145, or about 145 to about 165.

Thus, is some aspects, the PPG is a branched PPG. Branched PPGs have three to ten PPG chains emanating from a central core group. In certain aspects, the PPG moiety is a monodisperse polyethylene glycol. In the context of the present disclosure, a monodisperse polyethylene glycol (mdPPG) is a PPG that has a single, defined chain length and molecular weight. mdPEGs are typically generated by separation from the polymerization mixture by chromatography. In certain formulae, a monodisperse PPG moiety is assigned the abbreviation mdPPG.

In some aspects, the PPG is a Star PPG. Star PPGs have 10 to 100 PPG chains emanating from a central core group. In some aspects, the PPG is a Comb PPG. Comb PPGs have multiple PPG chains normally grafted onto a polymer backbone.

In certain aspects, the PPG has a molar mass (e.g., a number average molecular weight (Mn)) between about 1000 g/mol and about 2000 g/mol, between about 2000 g/mol and about 3000 g/mol, between about 3000 g/mol to about 4000 g/mol, between about 4000 g/mol and about 5000 g/mol, between about 5000 g/mol and about 6000 g/mol, between about 6000 g/mol and about 7000 g/mol, or between 7000 g/mol and about 8000 g/mol.

In some aspects, the PPG is PPG₁₀₀, PPG₂₀₀, PPG₃₀₀, PPG₄₀₀, PPG₅₀₀, PPG₆₀₀, PPG₇₀₀, PPG₈₀₀, PPG₉₀₀, PPG₁₀₀₀, PPG₁₁₀₀, PPG₁₂₀₀, PPG₁₃₀₀, PPG₁₄₀₀, PPG₁₅₀₀, PPG₁₆₀₀, PPG₁₇₀₀, PPG₁₈₀₀, PPG₁₉₀₀, PPG₂₀₀₀, PPG₂₁₀₀, PPG₂₂₀₀, PPG₂₃₀₀, PPG₂₄₀₀, PPG₂₅₀₀, PPG₁₆₀₀, PPG₁₇₀₀, PPG₁₈₀₀, PPG₁₉₀₀, PPG₂₀₀₀, PPG₂₁₀₀, PPG₂₂₀₀, PPG₂₃₀₀, PPG₂₄₀₀, PPG₂₅₀₀, PPG₂₆₀₀, PPG₂₇₀₀, PPG₂₈₀₀, PPG₂₉₀₀, PPG₃₀₀₀, PPG₃₁₀₀, PPG₃₂₀₀, PPG₃₃₀₀, PPG₃₄₀₀, PPG₃₅₀₀, PPG₃₆₀₀, PPG₃₇₀₀, PPG₃₈₀₀, PPG₃₉₀₀, PPG₄₀₀₀, PPG₄₁₀₀, PPG₄₂₀₀, PPG₄₃₀₀, PPG₄₄₀₀, PPG₄₅₀₀, PPG₄₆₀₀, PPG₄₇₀₀, PPG₄₈₀₀, PPG₄₉₀₀, PPG₅₀₀₀, PPG₅₁₀₀, PPG₅₂₀₀, PPG₅₃₀₀, PPG₅₄₀₀, PPG₅₅₀₀, PPG₅₆₀₀, PPG₅₇₀₀, PPG₅₈₀₀, PPG₅₉₀₀, PPG₆₀₀₀, PPG₆₁₀₀, PPG₆₂₀₀, PPG₆₃₀₀, PPG₆₄₀₀, PPG₆₅₀₀, PPG₆₆₀₀, PPG₆₇₀₀, PPG₆₈₀₀, PPG₆₉₀₀, PPG₇₀₀₀, PPG₇₁₀₀, PPG₇₂₀₀, PPG₇₃₀₀, PPG₇₄₀₀, PPG₇₅₀₀, PPG₇₆₀₀, PPG₇₇₀₀, PPG₇₈₀₀, PPG₇₉₀₀, or PPG₈₀₀₀. In some aspects, the PPG is PPG₅₀₀₀. In some aspects, the PPG is PPG₆₀₀₀. In some aspects, the PPG is PPG₄₀₀₀.

In some aspects, the PPG is monodisperse, e.g., mPPG₁₀₀, mPPG₂₀₀, mPPG₃₀₀, mPPG₄₀₀, mPPG₅₀₀, mPPG₆₀₀, mPPG₇₀₀, mPPG₈₀₀, mPPG₉₀₀, mPPG₁₀₀₀, mPPG₁₁₀₀, mPPG₁₂₀₀, mPPG₁₃₀₀, mPPG₁₄₀₀, mPPG₁₅₀₀, mPPG₁₆₀₀, mPPG₁₇₀₀, mPPG₁₈₀₀, mPPG₁₉₀₀, mPPG₂₀₀₀, mPPG₂₁₀₀, mPPG₂₂₀₀, mPPG₂₃₀₀, mPPG₂₄₀₀, mPPG₂₅₀₀, mPPG₁₆₀₀, mPPG₁₇₀₀, mPPG₁₈₀₀, mPPG₁₉₀₀, mPPG₂₀₀₀, mPPG₂₁₀₀, mPPG₂₂₀₀, mPPG₂₃₀₀, mPPG₂₄₀₀, mPPG₂₅₀₀, mPPG₂₆₀₀, mPPG₂₇₀₀, mPPG₂₈₀₀, mPPG₂₉₀₀, mPPG₃₀₀₀, mPPG₃₁₀₀, mPPG₃₂₀₀, mPPG₃₃₀₀, mPPG₃₄₀₀, mPPG₃₅₀₀, mPPG₃₆₀₀, mPPG₃₇₀₀, mPPG₃₈₀₀, mPPG₃₉₀₀, mPPG₄₀₀₀, mPPG₄₁₀₀, mPPG₄₂₀₀, mPPG₄₃₀₀, mPPG₄₄₀₀, mPPG₄₅₀₀, mPPG₄₆₀₀, mPPG₄₇₀₀, mPPG₄₈₀₀, mPPG₄₉₀₀, mPPG₅₀₀₀, mPPG₅₁₀₀, mPPG₅₂₀₀, mPPG₅₃₀₀, mPPG₅₄₀₀, mPPG₅₅₀₀, mPPG₅₆₀₀, mPPG₅₇₀₀, mPPG₅₈₀₀, mPPG₅₉₀₀, mPPG₆₀₀₀, mPPG₆₁₀₀, mPPG₆₂₀₀, mPPG₆₃₀₀, mPPG₆₄₀₀, mPPG₆₅₀₀, mPPG₆₆₀₀, mPPG₆₇₀₀, mPPG₆₈₀₀, mPPG₆₉₀₀, mPPG₇₀₀₀, mPPG₇₁₀₀, mPPG₇₂₀₀, mPPG₇₃₀₀, mPPG₇₄₀₀, mPPG₇₅₀₀, mPPG₇₆₀₀, mPPG₇₇₀₀, m PPG₇₈₀₀, mPPG₇₉₀₀, or mPPG₈₀₀₀. In some aspects, the mPPG is mPPG₅₀₀₀. In some aspects, the mPPG is mPPG₆₀₀₀. In some aspects, the mPPG is mPPG₄₀₀₀.

b. Cationic Carrier

In some aspects, the cationic carrier units of the present disclosure comprise at least one cationic carrier moiety. The term “cationic carrier” refers to a moiety or portion of a cationic carrier unit of the present disclosure comprising a plurality of positive charges that can interact and bind electrostatically an anionic payload (or an anionic carrier attached to a payload). In some aspects, the number of positive charges or positively charged groups on the cationic carrier is similar to the number of negative charges or negatively charged groups on the anionic payload (or an anionic carrier attached to a payload). In some aspects, the cationic carrier comprises a biopolymer, e.g., a peptide (e.g., a polylysine).

In some aspects, the cationic carrier comprises one or more basic amino acids (e.g., lysine, arginine, histidine, or a combination thereof). In some aspects, the cationic carrier comprises at least about three, at least about four, at least about five, at least about six, at least about seven, at least about eight, at least about nine, at least about ten, at least about 11, at least about 12, at least about 13, at least about 14, at least about 15, at least about 16, at least about 17, at least about 18, at least about 19, at least about 20, at least about 21, at least about 22, at least about 23, at least about 24, at least about 25, at least about 26, at least about 27, at least about 28, at least about 29, at least about 30, at least about 31, at least about 32, at least about 33, at least about 34, at least about 35, at least about 36, at least about 37, at least about 38, at least about 39, at least about 40, at least about 41, at least about 42, at least about 43, at least about 44, at least about 45, at least about 46, at least about 47, at least about 48, at least about 49, at least about 50, at least about 51, at least about 52, at least about 53, at least about 54, at least about 55, at least about 56, at least about 57, at least about 58, at least about 59, at least about 60, at least about 61, at least about 62, at least about 63, at least about 64, at least about 65, at least about 66, at least about 67, at least about 68, at least about 69, at least about 70, at least about 71, at least about 72, at least about 73, at least about 74, at least about 75, at least about 76, at least about 77, at least about 78, at least about 79, at least about 80 basic amino acids, e.g., lysines, arginines, or combinations thereof.

In some aspects, the cationic carrier unit comprises at least about 30 basic amino acids, e.g., lysines. In some aspects, the cationic carrier unit comprises at least about 35 basic amino acids, e.g., lysines. In some aspects, the cationic carrier unit comprises at least about 40 basic amino acids, e.g., lysines. In some aspects, the cationic carrier unit comprises at least about 45 basic amino acids, e.g., lysines. In some aspects, the cationic carrier unit comprises at least about 50 basic amino acids, e.g., lysines. In some aspects, the cationic carrier unit comprises at least about 55 basic amino acids, e.g., lysines. In some aspects, the cationic carrier unit comprises at least about 60 basic amino acids, e.g., lysines. In some aspects, the cationic carrier unit comprises at least about 65 basic amino acids, e.g., lysines. In some aspects, the cationic carrier unit comprises at least about 70 basic amino acids, e.g., lysines. In some aspects, the cationic carrier unit comprises at least about 75 basic amino acids, e.g., lysines. In some aspects, the cationic carrier unit comprises at least about 80 basic amino acids, e.g., lysines.

In some aspects, the cationic carrier unit comprises about 30 to about 1000, about 30 to about 900, about 30 to about 800, about 30 to about 700, about 30 to about 600, about 30 to about 500, about 30 to about 400, about 30 to about 300, about 30 to about 200, about 30 to about 100, about 40 to about 1000, about 40 to about 900, about 40 to about 800, about 40 to about 700, about 40 to about 600, about 40 to about 500, about 40 to about 400, about 40 to about 300, about 40 to about 200, or about 40 to about 100 basic amino acids, e.g., lysines. In some aspects, the basic amino acids, e.g., lysines, are not modified such that they possess a quaternary amine (e.g., positive charge).

In some aspects, the cationic carrier unit comprises about 30 to about 100, about 30 to about 90, about 30 to about 80, about 30 to about 70, about 30 to about 60, about 30 to about 50, about 30 to about 40, about 40 to about 100, about 40 to about 90, about 40 to about 80, about 40 to about 70, about 40 to about 60, about 70 to about 80, about 75 to about 85, about 65 to about 75, about 65 to about 80, about 60 to about 85, or about 40 to about 500 basic amino acids, e.g., lysines.

In some aspects, the cationic carrier unit comprises about 100 to about 1000, about 100 to about 900, about 100 to about 800, about 100 to about 700, about 100 to about 600, about 100 to about 500, about 100 to about 400, about 100 to about 300, about 100 to about 200, about 200 to about 1000, about 200 to about 900, about 200 to about 800, about 200 to about 700, about 200 to about 600, about 200 to about 500, about 200 to about 400, about 200 to about 300, about 300 to about 1000, about 300 to about 900, about 300 to about 800, about 300 to about 700, about 300 to about 600, about 300 to about 500, about 300 to about 400, about 400 to about 1000, about 400 to about 900, about 400 to about 800, about 400 to about 700, about 400 to about 600, about 400 to about 500, about 500 to about 1000, about 500 to about 900, about 500 to about 800, about 500 to about 700, about 500 to about 600, about 600 to about 1000, about 600 to about 900, about 600 to about 800, about 600 to about 700, about 700 to about 1000, about 700 to about 900, about 700 to about 800, about 800 to about 1000, about 800 to about 900, or about 900 to about 1000 basic amino acids, e.g., lysines.

In some aspects, the number of basic amino acids, e.g., lysines, arginines, histidines, or combinations thereof, can be adjusted based on the length of the anionic payload and/or charge of the anionic payload (e.g., single stranded nucleic acid or double stranded nucleic acid). For example, an anionic payload with a longer sequence can be paired with higher number of basic amino acids, e.g., lysines. In some aspects, the number of basic amino acids, e.g., lysines, in the cationic carrier unit can be calculated so that the molar ratio of protonated amine in polymer to phosphate in an anionic payload, e.g., oligonucleotide, e.g., antimir (N/P ratio) is about 1.0, about 1.1, about 1.2, about 1.3, about 1.4, about 1.5, about 1.6, about 1.7, about 1.8, about 1.9, about 2.0, about 2.1, about 2.2, about 2.3, about 2.4, about 2.5, about 2.6, about 2.7, about 2.8, about 2.9, or about 3. In some aspects, the number of basic amino acids, e.g., lysines, in the cationic carrier unit is calculated so that the molar ratio of protonated amine in polymer to phosphate in an anionic payload, e.g., oligonucleotide, e.g., antimir (N/P ratio) is about 1.3 to about 1.7, e.g., about 1.5. In some aspects, the number of basic amino acids, e.g., lysines, in the cationic carrier unit is calculated so that the molar ratio of protonated amine in polymer to phosphate in an anionic payload, e.g., oligonucleotide, e.g., antimir (N/P ratio) is about 1.4. In some aspects, the number of basic amino acids, e.g., lysines, in the cationic carrier unit is calculated so that the molar ratio of protonated amine in polymer to phosphate in an anionic payload, e.g., oligonucleotide, e.g., antimir (N/P ratio) is about 1.6. In some aspects, the number of basic amino acids, e.g., lysines, in the cationic carrier unit is calculated so that the molar ratio of protonated amine in polymer to phosphate in an anionic payload, e.g., oligonucleotide, e.g., antimir (N/P ratio) is about 1.3. In some aspects, the number of basic amino acids, e.g., lysines, in the cationic carrier unit is calculated so that the molar ratio of protonated amine in polymer to phosphate in an anionic payload, e.g., oligonucleotide, e.g., antimir (N/P ratio) is about 1.7.

In some aspects, the number of basic amino acids, e.g., lysines, in the cationic carrier unit is calculated so that the molar ratio of protonated amine in polymer to phosphate in an anionic payload, e.g., cholesterol conjugated siRNA (N/P ratio) is about 1.0.

In some aspects, the number of basic amino acids, e.g., lysines, in the cationic carrier unit is calculated so that the molar ratio of protonated amine in polymer to phosphate in an anionic payload, e.g., siRNA (N/P ratio) is about 2.0.

A person of skill in the art would understand that since a role of the cationic carrier moiety is to neutralize negative charges on the payload (e.g., negative changes in the phosphate backbone of an antisense oligonucleotide) via electrostatic interaction, in some aspects (e.g., when the payload is a nucleic acid such as an antimir), the length of the cationic carrier, number of positively charged groups on the cationic carrier, and distribution and orientation of charges present on the cationic carrier will depend on the length and charge distribution on the payload molecule.

In some aspects, the cationic carrier comprises between about 5 and about 10, between about 10 and about 15, between about 15 and about 20, between about 20 and about 25, between about 25 and about 30, between about 30 and about 35, between about 35 and about 40, between about 40 and about 45, between about 45 and about 50, between about 50 and about 55, between about 55 and about 60, between about 60 and about 65, between about and about 70, between about 70 and about 75, or between about 75 and about 80 basic amino acids. In some specific aspects, the positively charged carrier comprises between 30 and about 50 basic amino acids. In some specific aspects, the positively charged carrier comprises between 70 and about 80 basic amino acids.

In some aspects, the basic amino acid comprises arginine, lysine, histidine, or any combination thereof. In some aspects, the basic amino acid is a D-amino acid. In some aspects, the basic amino acid is an L-amino acid. In some aspects, the positively charged carrier comprises D-amino acids and L-amino acids. In some aspects, the basic amino comprises at least one unnatural amino acid or a derivative thereof. In some aspects, the basic amino acid is arginine, lysine, histidine, L-4-aminomethyl-phenylalanine, L-4-guanidine-phenylalanine, L-4-aminomethyl-N-isopropyl-phenylalanine, L-3-pyridyl-alanine, L-trans-4-aminomethylcyclohexyl-alanine, L-4-piperidinyl-alanine, L-4-aminocyclohexyl-alanine, 4-guanidinobutyric acid, L-2-amino-3-guanidinopropionic acid, DL-5-hydroxylysine, pyrrolysine, 5-hydroxy-L-lysine, methyllysine, hypusine, or any combination thereof. In a particular aspect, the positively charged carrier comprises about 40 lysines. In a particular aspect, the positively charged carrier comprises about 50 lysines. In a particular aspect, the positively charged carrier comprises about 60 lysines. In a particular aspect, the positively charged carrier comprises about 70 lysines. In a particular aspect, the positively charged carrier comprises about 80 lysines. In a particular aspect, the positively charged carrier comprises about 30 lysines. In a particular aspect, the positively charged carrier comprises about 40 lysines. In a particular aspect, the positively charged carrier comprises about 38 lysines. In a particular aspect, the positively charged carrier comprises about 32 lysines. In a particular aspect, the positively charged carrier comprises about 35 lysines.

In other aspects, the cationic carrier comprises a polymer or copolymer comprising at least three, at least four, at least five, at least six, at least seven, at least eight, at least nine, at least ten, at least 11, at least 12, at least 13, at least 14, at last 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 41, at least 42, at least 43, at least 44, at least 45, at least 46, at least 47, at least 48, at least 49, at least 50, at least 51, at least 52, at least 53, at least 54, at least 55, at least 56, at least 57, at least 58, at least 59, at least 60, at least 61, at least 62, at least 63, at least 64, at least 65, at least 66, at least 67, at least 68, at least 69, at least 70, at least 71, at least 72, at least 73, at least 74, at least 75, at least 76, at least 77, at least 78, at least 79, or at least 80 cationic groups (e.g., amino groups). In some aspects, the cationic carrier comprises a polymer or copolymer comprising between about 5 and about 10 cationic groups, between about 10 and about 15 cationic groups, between about 15 and about 20 cationic groups, between about 20 and about 25 cationic groups, between about 25 and about 30 cationic groups, between about 30 and about 35 cationic groups, between about 35 and about 40 cationic groups, between about 40 and about 45 cationic groups, between about 45 and about 50 cationic groups, between about 50 and about 55 cationic groups, between about 55 and about 60 cationic groups, between about 60 and about 65 cationic groups, between about 65 and about 70 cationic groups, between about 70 and about 75 cationic groups, or between about 45 and about 50 cationic groups (e.g., amino groups). In some specific aspects, the cationic carrier comprises a polymer or copolymer comprising between 30 and about 50 cationic groups (e.g., amino groups). In some specific aspects, the cationic carrier comprises a polymer or copolymer comprising between 70 and about 80 cationic groups (e.g., amino groups). In some aspects, the polymer or copolymer is an acrylate, a polyalcohol, or a polysaccharide.

In some aspects, the cationic carrier moiety binds to a single payload molecule. In other aspects, a cationic carrier moiety can bind to multiple payload molecules, which can be identical or different.

In some aspects, the positive charges of the cationic carrier moiety and negative charges of a nucleic acid payload are at an ionic ratio of about 5:1, about 4:1, about 3:1, about 2.9:1, about 2.8:1, about 2.7:1, about 2.6:1, about 2.5:1, about 2.4:1, about 2.3:1, about 2.2:1, about 2.1:1, about 2:1, about 1.9:1, about 1.8:1, about 1.7:1, about 1.6:1, about 1.5:1, about 1.4:1, about 1.3:1, about 1.2:1, about 1.1:1, about 1:1, about 1:1.1, about 1:1.2, about 1:1.3, about 1:1.4, about 1:1.5, about 1:1.6, about 1:1.7, about 1:1.8, about 1:1.9, about 1:2, about 1:2.1, about 1:2.2, about 1:2.3, about 1:2.4, about 1:2.5, about 1:2.6, about 1:2.7, about 1:2.8, about 1:2.9, about 1:3, about 1:4, or about 1:5. In some aspects, the positive charges of the cationic carrier moiety and the negative charged of the nucleic acid payload are at a charge ratio of 1:1. In some aspects, the positive charges of the cationic carrier moiety and the negative charged of the nucleic acid payload are at a charge ratio of 2:1. In some aspects, the positive charges of the cationic carrier moiety and the negative charges of the nucleic acid payload are at a charge ratio of 3:2. In some aspects, the positive charges of the cationic carrier moiety and the negative charges of the nucleic acid payload are at a charge ratio of 2:3. In some aspects, the positive charges of the cationic carrier moiety and the negative charged of the nucleic acid payload are at a charge ratio of 1:1.

In some aspects, the cationic carrier moiety has a free terminus wherein the end group is a reactive group. In some aspects, the cationic carrier moiety has a free terminus (e.g., the C-terminus in a poly-lysine cationic carrier moiety) wherein the end group is an amino (—NH₂) group. In some aspects, the cationic carrier moiety has a free terminus wherein the end group is a sulfhydryl group. In some aspects, the reactive group of the cationic carrier moiety is attached to a hydrophobic moiety, e.g., a nicotinic acid derivative hydrophobic moiety.

c. Crosslinking Moiety

In some aspects, the cationic carrier units of the present disclosure comprise at least one crosslinking moiety. The term “crosslinking moiety” refers to a moiety or portion of a polymer block comprising a plurality of agents that are capable of forming crosslinks. In some aspects, the number of agents that are capable of forming crosslinks comprises an amino acid with a side chain of a crosslinking agent. In some aspects, the CM comprises a biopolymer, e.g., a peptide (e.g., a polylysine) linked to a crosslinking agent.

In some aspects, the crosslinking moiety comprises one or more amino acids (e.g., lysine, arginine, histidine, or a combination thereof). In some aspects, the crosslinking moiety comprises at least about three, at least about four, at least about five, at least about six, at least about seven, at least about eight, at least about nine, at least about ten, at least about 11, at least about 12, at least about 13, at least about 14, at least about 15, at least about 16, at least about 17, at least about 18, at least about 19, at least about 20, at least about 21, at least about 22, at least about 23, at least about 24, at least about 25, at least about 26, at least about 27, at least about 28, at least about 29, or at least about 30 amino acids, e.g., lysines, arginines, or combinations thereof, each of which is linked to a crosslinking agent.

In some aspects, the lysines of the crosslinking moeity possess a neutral charge (e.g., contain a tertiary amine). In some aspects, lysines of the crosslinking moiety contain a thiol (e.g., lysine-thiol) and a tertiary amine, such that the lysines possess a neutral charge. In some aspects, the crosslinking moiety forms a crosslink through the tertiary amine. In some aspects, the crosslinking moiety forms a crosslink through the thiol. As used herein, “lysine” in the context of the crosslinking moiety, refers to lysines with a neutral charge (e.g., containing a tertiary amine), such that the lysines of the crosslinking moiety do not contribute to the overall charge of the carrier unit. In some aspects, the lysines of the crosslinking moiety are linked to a crosslinking agent through an amide bond.

In some aspects, the crosslinking moiety comprises at least about 5 amino acids, e.g., lysines, each of which is linked to a crosslinking agent. In some aspects, the crosslinking moiety comprises at least about 10 amino acids, e.g., lysines, each of which is linked to a crosslinking agent. In some aspects, the crosslinking moiety comprises at least about 11 amino acids, e.g., lysines, each of which is linked to a crosslinking agent. In some aspects, the crosslinking moiety comprises at least about 12 amino acids, e.g., lysines, each of which is linked to a crosslinking agent. In some aspects, the crosslinking moiety comprises at least about 13 amino acids, e.g., lysines, each of which is linked to a crosslinking agent. In some aspects, the crosslinking moiety comprises at least about 14 amino acids, e.g., lysines, each of which is linked to a crosslinking agent. In some aspects, the crosslinking moiety comprises at least about 15 amino acids, e.g., lysines, each of which is linked to a crosslinking agent. In some aspects, the crosslinking moiety comprises at least about 16 amino acids, e.g., lysines, each of which is linked to a crosslinking agent. In some aspects, the crosslinking moiety comprises at least about 17 amino acids, e.g., lysines, each of which is linked to a crosslinking agent. In some aspects, the crosslinking moiety comprises at least about 18 amino acids, e.g., lysines, each of which is linked to a crosslinking agent. In some aspects, the crosslinking moiety comprises at least about 19 amino acids, e.g., lysines, each of which is linked to a crosslinking agent. In some aspects, the crosslinking moiety comprises at least about 20 amino acids, e.g., lysines, each of which is linked to a crosslinking agent.

In some aspects, a crosslinking agent is a thiol. In some aspects, a crosslinking agent is a thiol derivative.

c. Hydrophobic Moiety

In some aspects, the cationic carrier units of the present disclosure comprise at least one hydrophobic moiety. The term “hydrophobic moiety,” as used herein, refers to a molecular entity that can, e.g., (i) complement the therapeutic or prophylactic activity of the payload, (ii) modulate the therapeutic or prophylactic activity of the payload, (iii) function as a therapeutic and/or prophylactic agent in the target tissue or target cells, (iv) facilitate the transport of the cationic carrier unit across a physiological barrier, e.g., the BBB and/or the plasma membrane, (v) improve the homeostasis of the target tissue or target cell, (vi) contribute positively charges groups to the cationic carried moiety, or (vii) any combination thereof.

In some aspects, the lysines of the hydrophobic moiety possess a neutral charge (e.g., contain a tertiary amine). In some aspects, lysines of the hydrophobic moiety contain a thiol (e.g., lysine-thiol) and a tertiary amine, such that the lysines possess a neutral charge. In some aspects, the hydrophobic moiety is linked to a nicotinic acid derivative through the tertiary amine (e.g., a neutrally charged amine), such that the lysines of the hydrophobic moiety do not contribute to the overall charge of the carrier unit. In some aspects, the hydrophobic moiety is linked to a nicotinic acid derivative through the thiol, such that the lysines of the hydrophobic moiety do not contribute to the overall charge of the carrier unit. In some aspects, the lysines of the hydrophobic moiety are linked to a nicotinic acid derivative through an amide bond.

In some aspects, the hydrophobic moiety is capable of modulating, e.g., an immune response, an inflammatory response, or a tissue microenvironment.

In some aspects, a hydrophobic moiety capable of modulating an immune response can comprise, e.g., tyrosine or dopamine. Tyrosine can be transformed into L-DOPA, and then converted to dopamine via a 2-step enzymatic reaction. Normally, dopamine levels are low in Parkinson's disease patients. Therefore, in some aspects, tyrosine is a hydrophobic moiety in cationic carrier units used for the treatment of Parkinson's disease. Tryptophan can be converted to serotonin, a neurotransmitter thought to play a role in appetite, emotions, and motor, cognitive, and autonomic functions. Accordingly, in some aspects, cationic carrier units of the present disclosure used for the treatment of disease or conditions related to low serotonin levels comprise tryptophan as a hydrophobic moiety.

In some aspects, a hydrophobic moiety can modulate a tumor microenvironment in a subject with a tumor, for example, by inhibiting or reducing hypoxia in the tumor microenvironment.

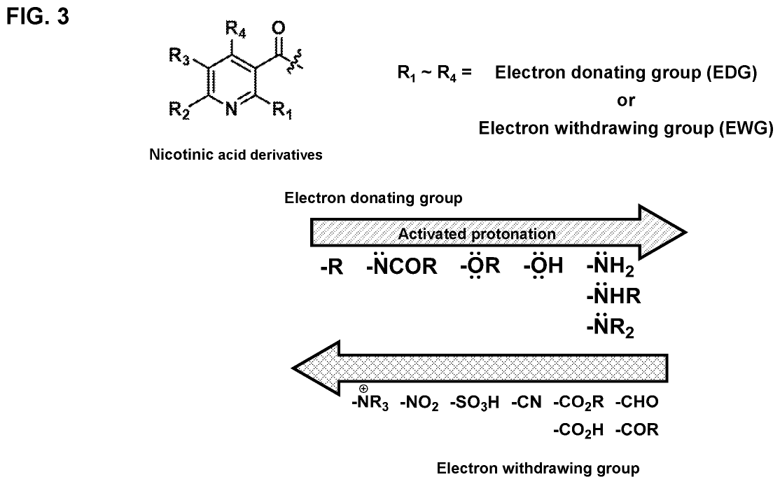

In some aspects, the nicotinic acid derivative comprises an electron donating group (EDG) or an electron withdrawing group (EWG). Exemplary EDG and EWG groups are shown in FIG. 3. In some aspects, the cationic carrier does not comprise a nicotinic acid residue.

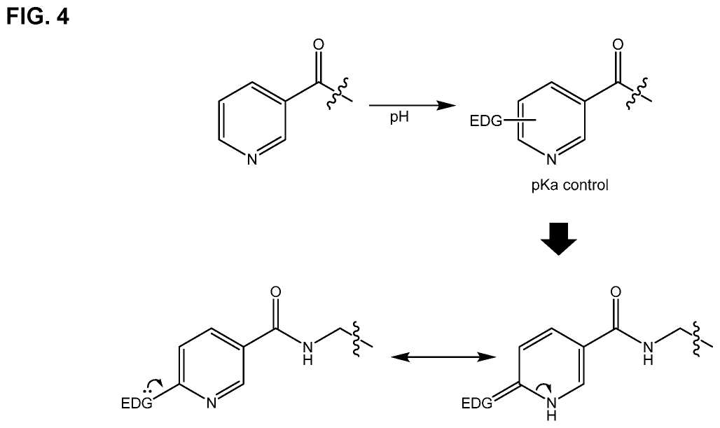

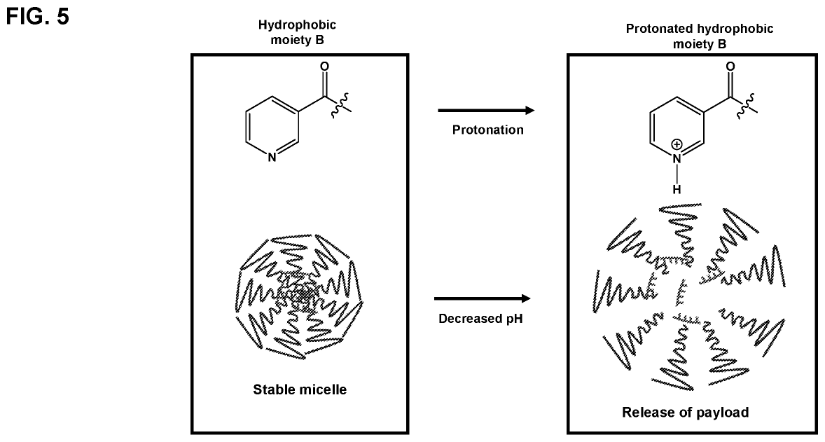

It was discovered that EDG or EWG can be attached to a nicotinic acid residue to control the total pKa of the micelle. For example, FIG. 4 shows the principle of pKa control of nicotinic acid derivatives by an EDG. At a different pH, the protonation of pyridine ring can be controlled by EDG. FIG. 5 shows how hydrophobic moiety B changes with the pH. When the pH is lower than the pKa of the hydrophobic moiety, the hydrophobic moiety is protonated and increases its hydrophilicity. The changes of hydrophobicity in the hydrophobic moiety affect the micelle stability and allow the payload to be released at a low pH. The changes in pKa of the hydrophobic moiety affect the micellar stability at different pH environments and enable the controlling of payload release. A micelle comprising a hydrophobic moiety (e.g., a nicotinic acid derivative) with a low pKa is useful in immediate-release drugs, whereas a micelle comprising a hydrophobic moiety (e.g., a nicotinic acid derivative) with a high pKa is useful in sustained-release drugs.

In some aspects, the hydrophobic moiety (e.g., a nicotinic acid derivative) has a pKa of less than 5.7. In some aspects, the hydrophobic moiety (e.g., a nicotinic acid derivative) has a pKa of greater than 5.7.

In some aspects, a micelle comprises a hydrophobic moiety (e.g., nicotinic acid derivative) with a pKa that allows the payload to be released immediately upon administration. In some aspects, a micelle comprises a hydrophobic moiety (e.g., nicotinic acid) with a pKa that allows the payload to be released slowly upon administration.

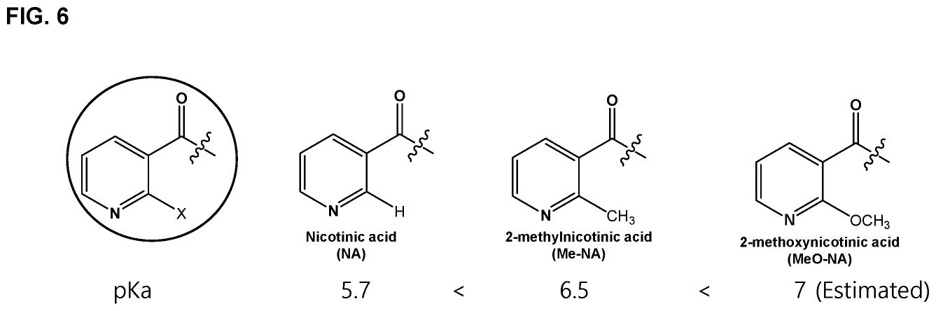

In some aspects, the nicotinic acid derivative comprises 2-methylnicotinic acid (Me-NA), 2-methoxynicotinic acid (MeQ-NA), or any combination thereof. In some aspects, the nicotinic acid derivative comprises 2-methylnicotinic acid (Me-NA). In some aspects, the nicotinic acid derivative comprises 2-methoxynicotinic acid (MeO-NA). FIG. 6 shows the pKa levels of nicotinic acid (NA) compared to nicotinic acid derivative, e.g., 2-methylnicotinic acid (Me-NA) and 2-methoxynicotinic acid (MeO-NA).

In some aspects, the nicotinic acid derivative comprises:

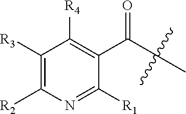

wherein one or more of R₁, R₂, R₃, and R₄ is an electron donating group. In some aspects, wherein each of R₁, R₂, R₃, and R₄ is an electron donating group. In some aspects, R₁, R₂, R₃, and R₄ are individually selected from the group consisting of hydrogen, C₁₋₄ alkyl, C₁₋₄ alkoxy, OH, amino, C₁₋₄ alkylamino, C₁₋₄ dialkylamino, C₁₋₄ alkylamido attached through the nitrogen (—NHC(O)alkyl), and C₁₋₄ alkylester attached through the ester oxygen (—OC(O)alkyl), provided that at least one of R₁, R₂, R₃, and R₄ is other than hydrogen. In some aspects, R₁ is C₁₋₄ alkyl (e.g., methyl) or C₁₋₄ alkoxy (e.g., methoxy). In some aspects, R₁ is C₁₋₄ alkyl (e.g., methyl) or C₁₋₄ alkoxy (e.g., methoxy) and R₂, R₃, and R₄ are each hydrogen.

In some aspects, the nicotinic acid derivative comprises:

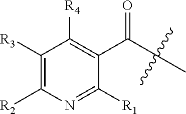

wherein one or more of R₁, R₂, R₃, and R₄ is an electron withdrawing group. In some aspects, each of R₁, R₂, R₃, and R₄ is an electron withdrawing group. In some aspects, R₁, R₂, R₃, and R₄ are individually selected from the group consisting of hydrogen, halo, CN, NO₂, quaternary C₁₋₄ alkylammonium, C₁₋₄ haloalkyl, —CHO, carboxy (—COOH), C₁₋₄ alkylamido attached through the carbonyl carbon (—C(O)NHalkyl), C₁₋₄ alkylester attached through the carbonyl carbon, (—C(O)Oalkyl), C₁₋₄ alkylcarbonyl (—C(O)alkyl), sulfonato (—SO₃H), and C₁₋₄ alkylsulfonato (—SO₃alkyl), provided that at least one of R₁, R₂, R₃, and R₄ is other than hydrogen.

In some aspects, the hydrophobic moiety is capable of inhibiting or reducing an inflammatory response.

In some aspects, the hydrophobic moiety is an amino acid (e.g., lysine) linked to a nicotinic acid derivative.

In some aspects, the hydrophobic moiety comprises at least about two, at least about three, at least about four, at least about five, at least about six, at least about seven, at least about eight, at least about nine, at least about ten, at least about 11, at least about 12, at least about 13, at least about 14, at least about 15, at least about 16, at least about 17, at least about 18, at least about 19, at least about 20, at least about 21, at least about 22, at least about 23, at least about 24, at least about 25, at least about 26, at least about 27, at least about 28, at least about 29, at least about 30, at least about 31, at least about 32, at least about 33, at least about 34, at least about 35, at least about 36, at least about 37, at least about 38, at least about 39, or at least about 40 amino acids (e.g., lysines), each of which is linked to nicotinic acid derivative. In some aspects, the hydrophobic moiety comprises about 30 amino acids (e.g., lysines), each of which is linked to a nicotinic acid derivative. In some aspects, the hydrophobic moiety comprises about 31 amino acids (e.g., lysines), each of which is linked to a nicotinic acid derivative. In some aspects, the hydrophobic moiety comprises about 32 amino acids (e.g., lysines), each of which is linked to a nicotinic acid derivative. In some aspects, the hydrophobic moiety comprises about 33 amino acids (e.g., lysines), each of which is linked to a nicotinic acid derivative. In some aspects, the hydrophobic moiety comprises about 34 amino acids (e.g., lysines), each of which is linked to a nicotinic acid derivative. In some aspects, the hydrophobic moiety comprises about 35 amino acids (e.g., lysines), each of which is linked to a nicotinic acid derivative. In some aspects, the hydrophobic moiety comprises about 36 amino acids (e.g., lysines), each of which is linked to a nicotinic acid derivative. In some aspects, the hydrophobic moiety comprises about 37 amino acids (e.g., lysines), each of which is linked to a nicotinic acid derivative. In some aspects, the hydrophobic moiety comprises about 38 amino acids (e.g., lysines), each of which is linked to a nicotinic acid derivative. In some aspects, the hydrophobic moiety comprises about 39 amino acids (e.g., lysines), each of which is linked to a nicotinic acid derivative. In some aspects, the hydrophobic moiety comprises about 40 amino acids (e.g., lysines), each of which is linked to a nicotinic acid derivative.

In some aspects the hydrophobic moiety comprises from about 20 to about 25 amino acids (e.g., lysines), each of which is linked to a nicotinic acid derivative, about 25 to about 30 amino acids (e.g., lysines), each of which is linked to a nicotinic acid derivative, about 30 to about 35 amino acids (e.g., lysines), each of which is linked to a nicotinic acid derivative, about 35 to about 40 amino acids (e.g., lysines), each of which is linked to a nicotinic acid derivative, about 40 to about 45 amino acids (e.g., lysines), each of which is linked to a nicotinic acid derivative, or about 45 to about 50 amino acids (e.g., lysines), each of which is linked to a nicotinic acid derivative. In some aspects the hydrophobic moiety comprises from about 20 to about 30 amino acids (e.g., lysines), each of which is linked to a nicotinic acid derivative, about 30 to about 40 amino acids (e.g., lysines), each of which is linked to a nicotinic acid derivative, about 40 to about 50 amino acids (e.g., lysines), each of which is linked to a nicotinic acid derivative, about 25 to about 35 amino acids (e.g., lysines), each of which is linked to a nicotinic acid derivative, or about 35 to about 45 amino acids (e.g., lysines), each of which is linked to a nicotinic acid derivative.

d. Targeting Moiety

In some aspects, the cationic carrier unit comprises a targeting moiety, which is linked to the water-soluble polymer optionally via a linker. As used herein, the term “targeting moiety” refers to a biorecognition molecule that binds to a specific biological substance or site. In some aspects, the targeting moiety is specific for a certain target molecule (e.g., a ligand targeting a receptor, or an antibody targeting a surface protein), tissue (e.g., a molecule that would preferentially carry the micelle to a specific organ or tissue, e.g., liver, brain, or endothelium), or facilitate transport through a physiological barrier (e.g., a peptide or other molecule that may facilitate transport across the brain blood barrier or plasma membrane).

For targeting a payload (e.g., a nucleotide molecule, e.g., an antisense oligonucleotide that binds to a microRNA) according to the present disclosure, a targeting moiety can be coupled to a cationic carrier unit, and therefore, to the external surface of a micelle, whereas the micelle has the payload entrapped within its core.

In some aspects, the targeting moiety is a targeting moiety capable of targeting the micelle of the present disclosure to a tissue. In some aspects, the tissue is liver, brain, kidney, lung, ovary, pancreas, thyroid, breast, stomach, or any combination thereof. In some aspects, the tissue is cancer tissue, e.g., liver cancer, brain cancer, kidney cancer, lung cancer, ovary cancer, pancreas cancer, thyroid cancer, breast cancer, stomach cancer, or any combination thereof.

In a specific aspect, the tissue is liver. In a specific aspect, the targeting moiety targeting liver is cholesterol. In other aspects, the targeting moiety targeting liver is a ligand that binds an asialoglycoprotein receptor-targeting moiety. In some aspects, the asialoglycoprotein receptor-targeting moiety comprises a GalNAc cluster. In some aspects, the GalNAc cluster is a monovalent, divalent, trivalent, or tetravalent GalNAc cluster.

In another aspect, the tissue is pancreas. In some aspects, the targeting moiety targeting pancreas comprises a ligand targeting αvβ3 integrin receptors on pancreatic cells. In some aspects, the targeting moiety comprises an arginylglycylaspartic acid (RGD) peptide sequence (L-Arginyl-Glycyl-L-Aspartic acid; Arg-Gly-Asp).

In some aspects, the tissue is a tissue in the central nervous system, e.g., neural tissue. In some aspects, the targeting moiety targeting the central nervous system is capable being transported by Large-neutral Amino Acid Transporter 1 (LAT1). LAT1 (SLC7A5) is a transporter for both the uptake of large neutral amino acids and a number of pharmaceutical drugs. LAT1 can transport drugs such as L-dopa or gabapentin.

In some aspects, a targeting moiety comprises glucose, e.g., D-glucose, which can bind to Glucose transporter 1 (or GLUT1) and cross BBB. GLUT1, also known as solute carrier family 2, facilitated glucose transporter member 1 (SLC2A1), is a uniporter protein that in humans is encoded by the SLC2Al gene. GLUT1 facilitates the transport of glucose across the plasma membranes of mammalian cells. This gene encodes a major glucose transporter in the mammalian blood-brain barrier.

In some aspects, a targeting moiety comprises galactose, e.g., D-galactose, which can bind to GLUT1 transporter to cross BBB. In some aspects, a targeting moiety comprises glutamic acid, which can bind to acetylcholinesterase inhibitor (AChEI) and/or EAATs inhibitors and cross BBB. Acetylcholinesterase is the enzyme that is the primary member of the cholinesterase enzyme family. An acetylcholinesterase inhibitor (AChEI) is the inhibitor that inhibits acetylcholinesterase from breaking down acetylcholine into choline and acetate, thereby increasing both the level and duration of action of the neurotransmitter acetylcholine in the central nervous system, autonomic ganglia and neuromuscular junctions, which are rich in acetylcholine receptors. Acetylcholinesterase inhibitors are one of two types of cholinesterase inhibitors; the other being butyryl-cholinesterase inhibitors.

In some aspects, a targeting moiety is GABA, which can bind to GABA receptors to cross BBB. The GABA receptors are a class of receptors that respond to the neurotransmitter gamma-aminobutyric acid (GABA), the chief inhibitory compound in the mature vertebrate central nervous system. There are two classes of GABA receptors: GABAA and GABAB. GABAA receptors are ligand-gated ion channels (also known as ionotropic receptors); whereas GABAB receptors are G protein-coupled receptors, also called metabotropic receptors.

In some aspects, a targeting moiety comprises tyrosine, which can bind to LAT1 and cross BBB. In some aspects, a targeting moiety comprises lysine, which can bind to LAT1 and cross BBB. In some aspects, a targeting moiety comprises glutamine, which can bind to LAT1 and cross BBB. In some aspects, a targeting moiety comprises phenylalanine, which can bind to GABA receptors, LAT1, CNS reverse transcriptase inhibitors, and/or dopamine (DA) receptors and cross BBB. Dopamine receptors are a class of G protein-coupled receptors that are prominent in the vertebrate central nervous system (CNS). Dopamine receptors activate different effectors through not only G-protein coupling, but also signaling through different protein (dopamine receptor-interacting proteins) interactions. The neurotransmitter dopamine is the primary endogenous ligand for dopamine receptors.

Dopamine receptors are implicated in many neurological processes, including motivation, pleasure, cognition, memory, learning, and fine motor control, as well as modulation of neuroendocrine signaling. Abnormal dopamine receptor signaling and dopaminergic nerve function is implicated in several neuropsychiatric disorders. Thus, dopamine receptors are common neurologic drug targets; antipsychotics are often dopamine receptor antagonists while psychostimulants are typically indirect agonists of dopamine receptors.

In some aspects, a targeting moiety comprises valine, which can bind to CNS reverse transcriptase inhibitors and cross BBB. In some aspects, a targeting moiety comprises tryptophan, which can bind to GABA receptors and/or CNS reverse transcriptase inhibitors and cross BBB. In some aspects, a targeting moiety comprises leucine, which can bind to GABA receptors and/or CNS reverse transcriptase inhibitors and cross BBB. In some aspects, a targeting moiety comprises methionine, which can bind to GABA receptors and/or CNS reverse transcriptase inhibitors and cross BBB. In some aspects, a targeting moiety comprises histidine, which can bind to GABA receptors and cross BBB. In some aspects, a targeting moiety comprises isoleucine, which can bind to CNS reverse transcriptase inhibitors and cross BBB. In some aspects, a targeting moiety comprises Glutathione, which can bind to GSH transporter and cross BBB. In some aspects, a targeting moiety comprises Glutathione-Met, which can bind to GSH transporter and cross BBB. In some aspects, a targeting moiety comprises Urea/Thiourea, which can bind to Nitric oxide synthase (NOS) and bind to BBB. In some aspects, a targeting moiety comprises NAD+/NADH, which is capable of crossing BBB by REDOX mechanism. In some aspects, a targeting moiety comprises purine and can cross BBB. Additional examples of targeting moieties for CNS targeting are shown in Sutera et al. (2016): Small endogenous molecules as moiety to improve targeting of CNS drugs, Expert Opinion on Drug Delivery, DOI: 10.1080/17425247.2016.1208651, which is incorporated herein by reference in its entirety.

In some aspects, the tissue targeted by a targeting moiety is a skeletal muscle. In some aspects, the targeting moiety targeting skeletal muscle is capable being transported by Large-neutral Amino Acid Transporter 1 (LAT1).

It is expressed in numerous cell types including T-cells, cancer cells and brain endothelial cells. LAT1 is consistently expressed at high levels in brain microvessel endothelial cells. Being a solute carrier located primarily in the BBB, targeting the micelles of the present disclosure to LAT1 allows delivery through the BBB. In some aspects, the targeting moiety targeting a micelle of the present disclosure to the LAT1 transporter is an amino acid, e.g., a branched-chain or aromatic amino acid. In some aspects, the amino acid is valine, leucine, and/or isoleucine. In some aspects, the amino acid is tryptophan and/or tyrosine. In some aspects, the amino acid is tryptophan. In other aspects, the amino acid is tyrosine.

In some aspects, the targeting moiety is a LAT1 ligand selected from tryptophan, tyrosine, phenylalanine, tryptophan, methionine, thyroxine, melphalan, L-DOPA, gabapentin, 3,5-I-diiodotyrosine, 3-iodo-I-tyrosine, fenclonine, acivicin, leucine, BCH, methionine, histidine, valine, or any combination thereof.

In some aspects, the LAT1 ligand is [1] 1-Phenylalanine, [2] o-Sarcolysin, [3] m-Sarcolysin. [4] Melphalan. [5] 2-Amino-2-norbornanecarboxylic acid (BCH). [6] (±)-2-Amino-1,2,3,4-tetrahydro-2-naphthoic acid, [7] dl-2-NAM-5, [8] dl-2-NAM-6, [9] dl-2-NAM-7, [10] dl-2-NAM-8, [11] dl-dechlorinated-NAM, [12] dl-1-NAM-7, [13] (±)-2-Aminoindane-2 carboxylic acid, [14] (±)-2-Aminobenzo-bicyclo-[2.2.1]heptane-2′-exo-carboxylic acid, [15] (±)-2-amino-(bis-2-chloroethyl)-5-aminoindane-2-carboxylic acid, [16] (+)-2-endo-amino-bis(2-chloroethyl)-7′-aminobenzobicyclo[2.2.1]heptane-2-exo-carboxylic acid, [17] 1-6-diazo-5-oxo-norleucine (1-DON), [18] Acivicin, [19] Azaserine, [20] Buthionine Sulfoximine (BSO), [21] 1-1-naphthylalanine, [22] o-benzyl-l-tyrosine, [23] 1-2-amino-nonanoic acid, [24] 1-Tyrosine, [25] α-methyltyrosine, [26] 1-DOPA, [27] α-methyldopa, [28] 3-o-methyldopa, [29] Droxidopa, [30] Carbidopa, [31] Dopamine, [32] Tyramine, [33] α-methylphenylalanine, [34] N-methylphenylalanine, [35] Phenylalanine methyl ester, [36] Gabapentin, [37] 3,3′-diiodothyronine, [38] 1-T3, [39] 3′,5′,3-triiodothyronine (r1-T3), or [40], 1-T4, or any combination thereof, as shown below.

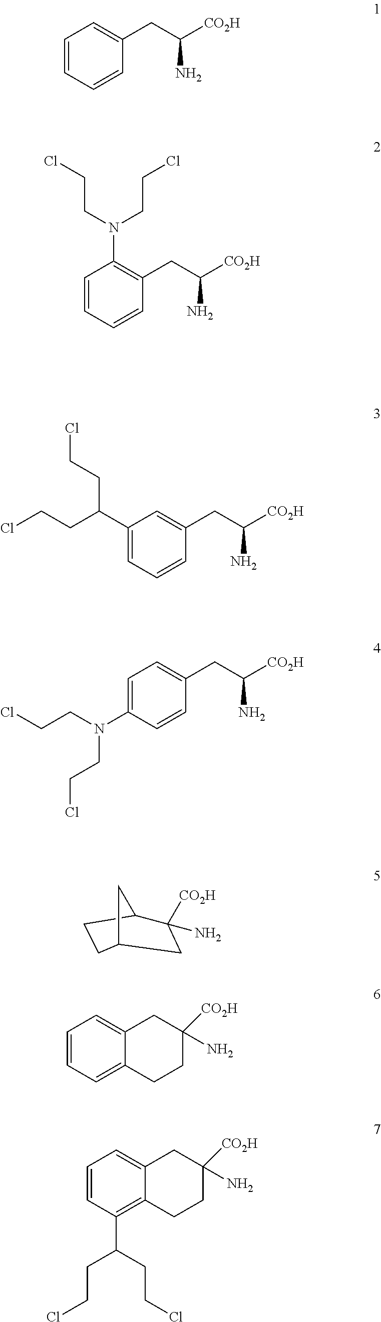

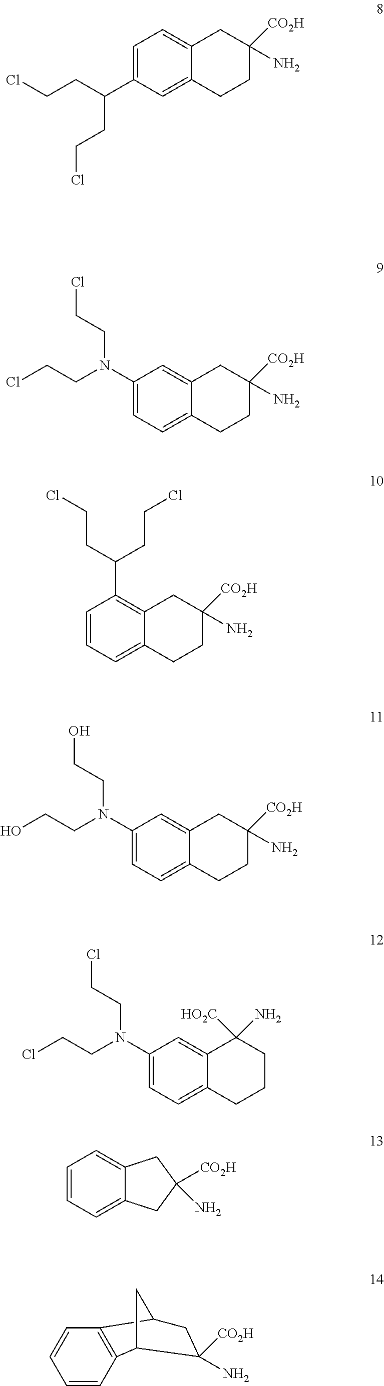

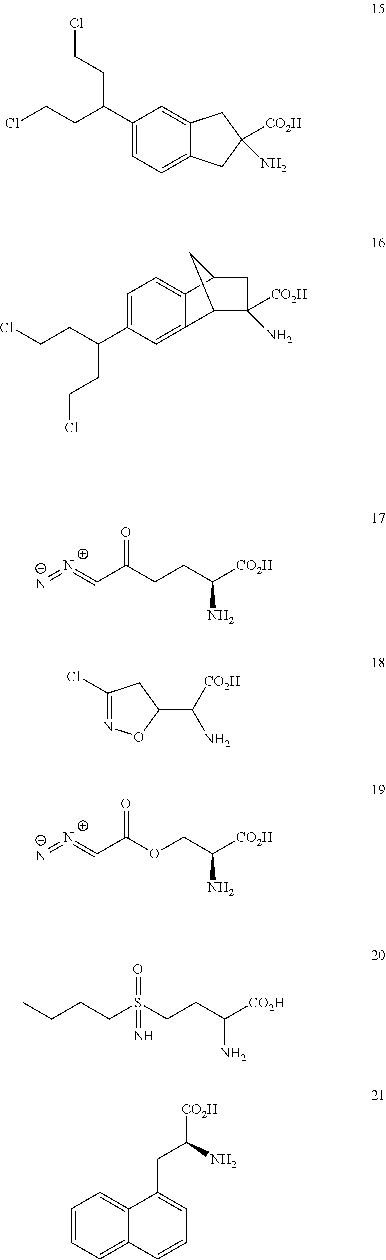

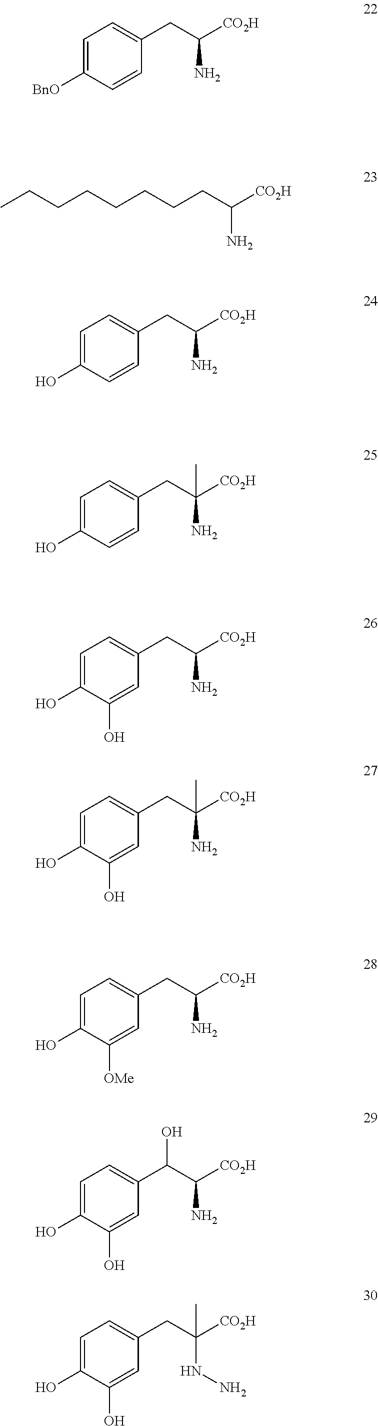

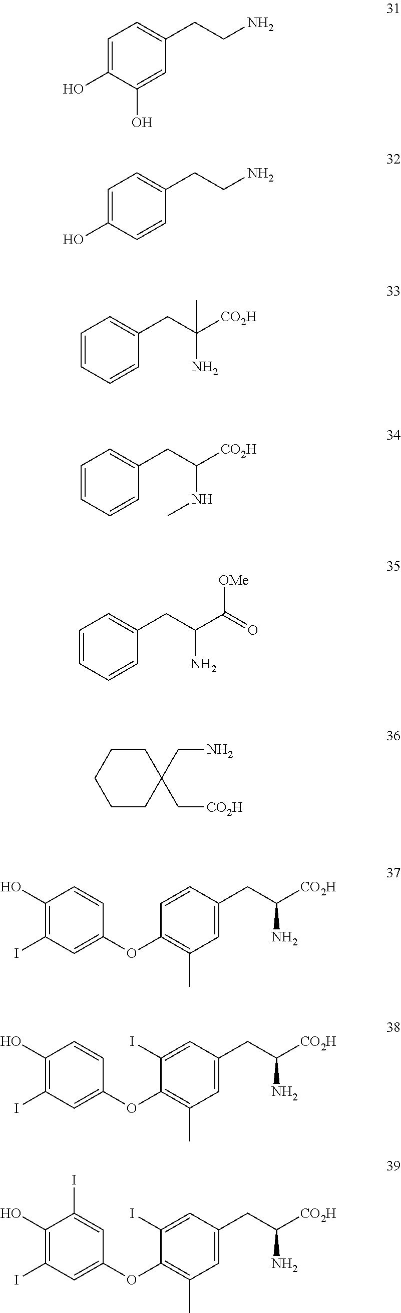

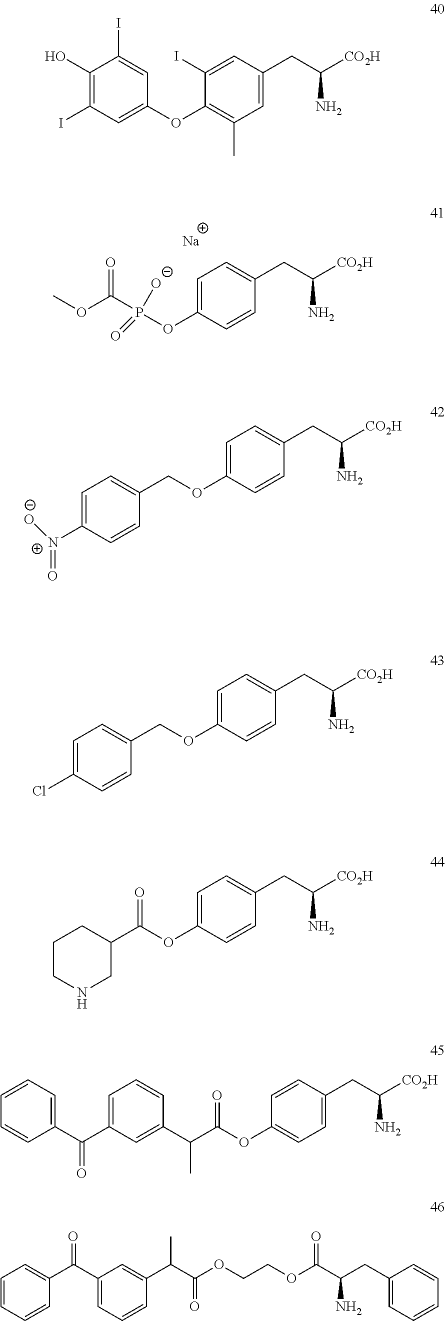

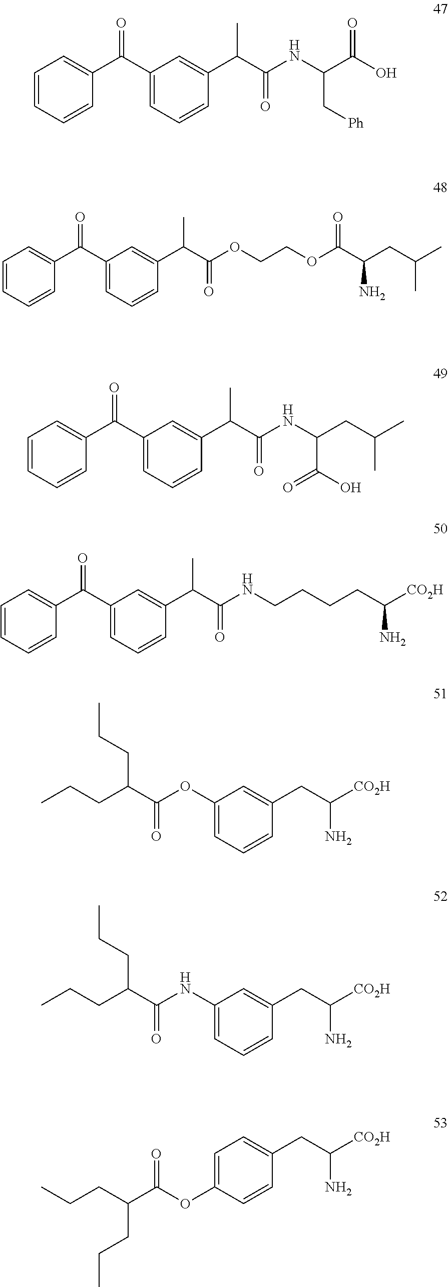

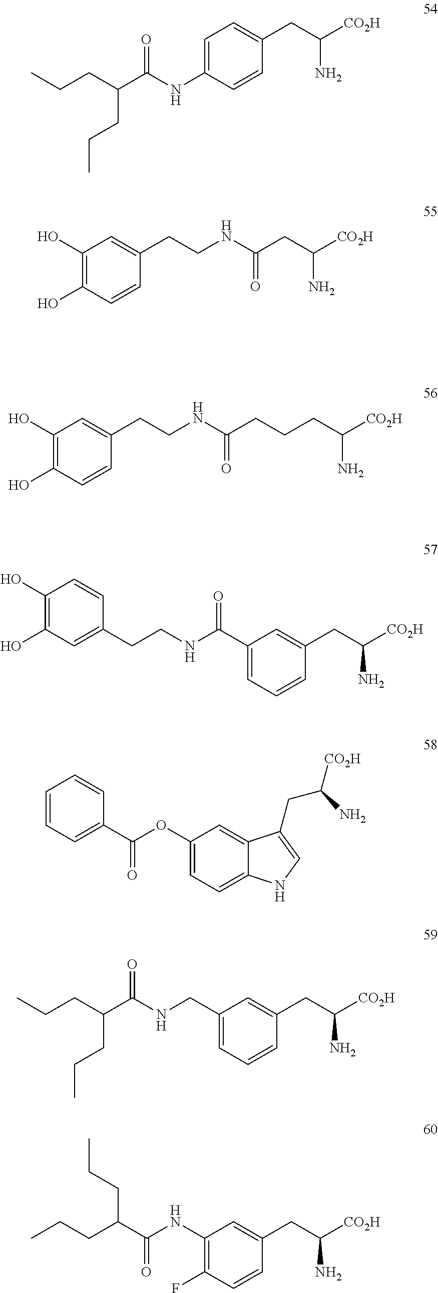

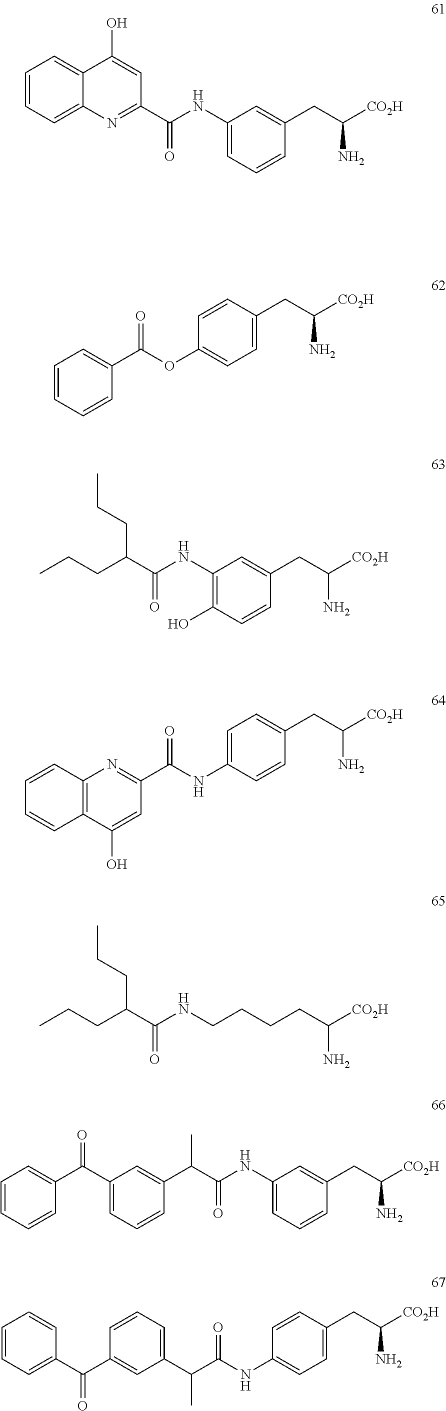

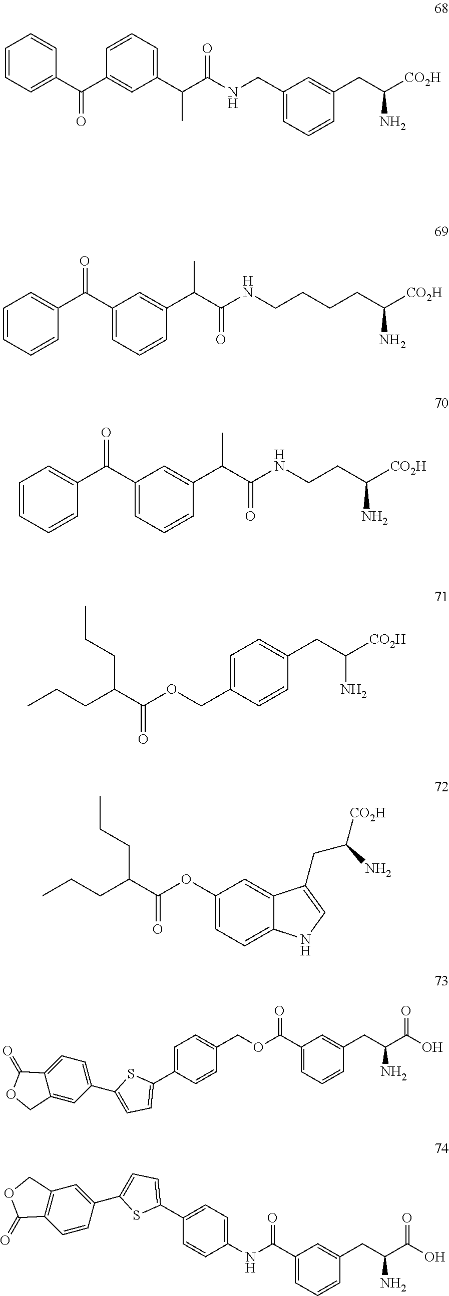

or any combination thereof.

In some aspects, the LAT1 ligand is a LAT1-targeting prodrug shown below.

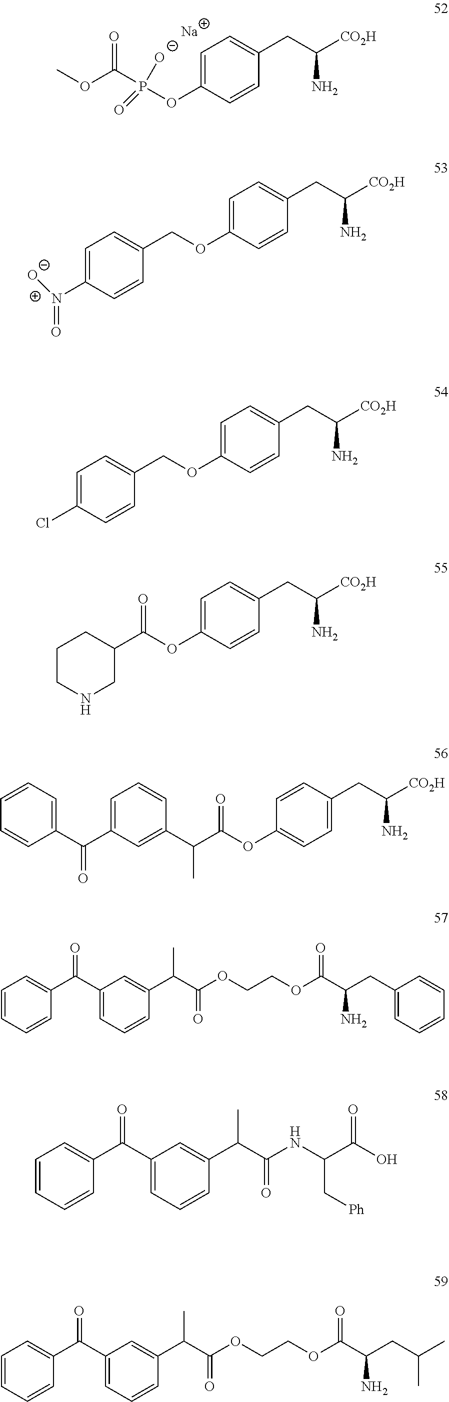

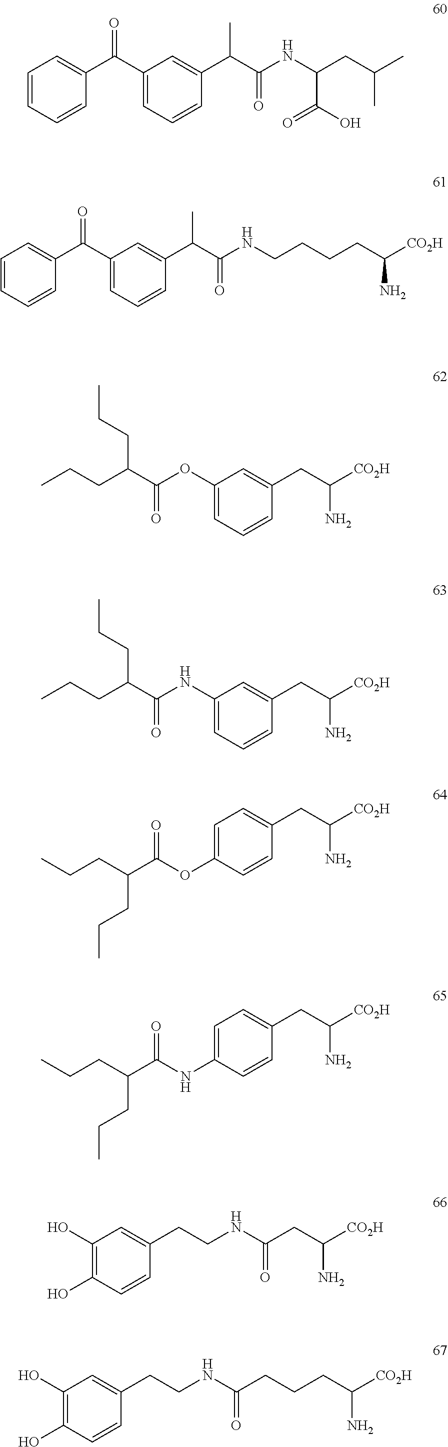

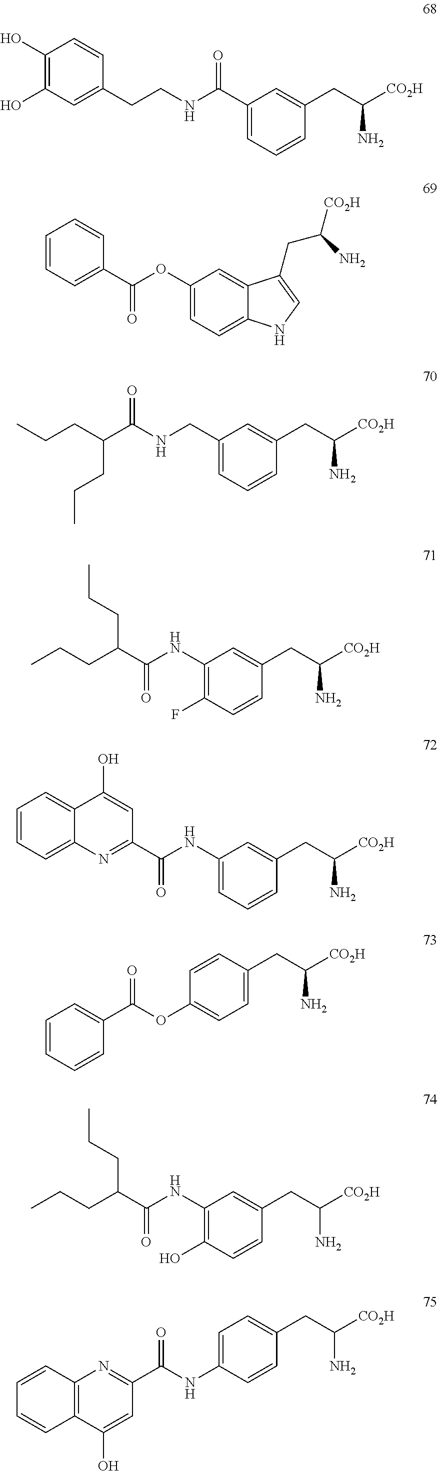

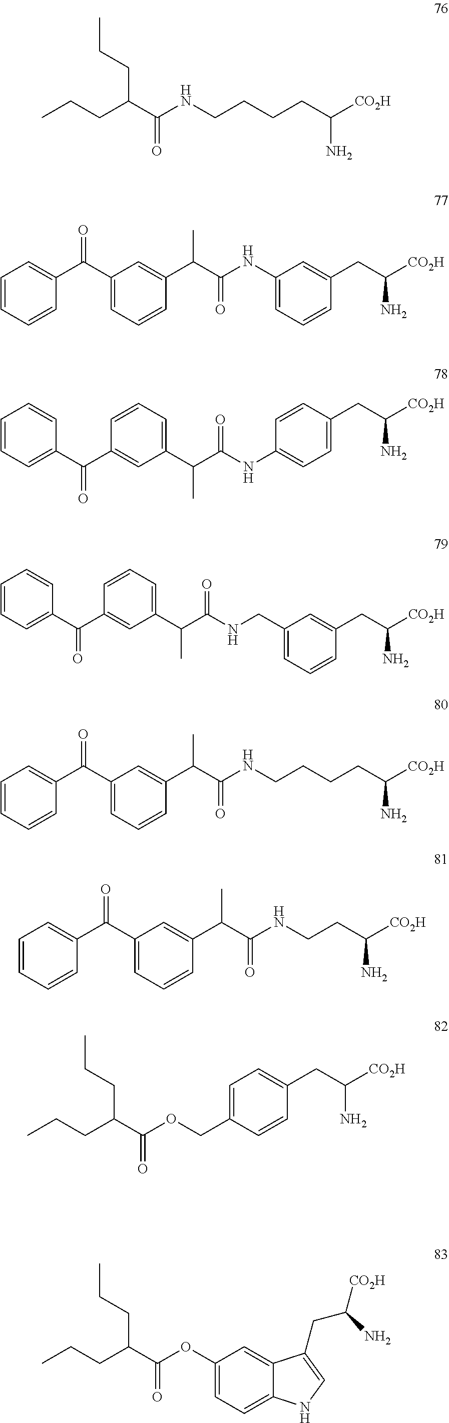

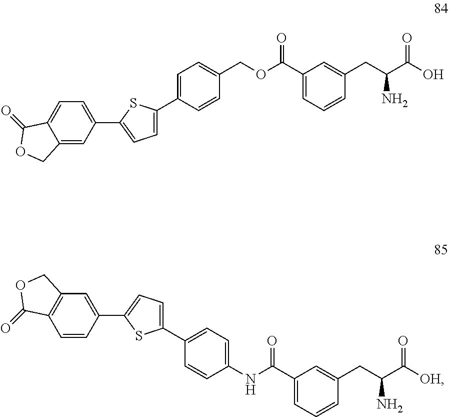 [SMILES: N[C@@H](Cc1cccc(C(=O)OCc2ccc(-c3ccc(-c4ccc5c(c4)COC5=O)s3)cc2)c1)C(=O)O ; N[C@@H](Cc1cccc(C(=O)Nc2ccc(-c3ccc(-c4ccc5c(c4)COC5=O)s3)cc2)c1)C(=O)O]

or any combination thereof.

See Singh & Ecker (2018) “Insights into the Structure, Function, and Ligand Discovery of the Large Neutral Amino Acid Transporter 1, LAT1,” Int. J. Mol. Sci. 19:1278; Geier et al. (2013) “Structure-based ligand discovery for the Large-neutral Amino Acid Transporter 1, LAT-1,” Proc. Natl. Acad. Sci. USA 110:5480-85; and Chien et al. (2018) “Reevaluating the Substrate Specificity of the L-type Amino Acid Transporter (LAT1),” J. Med. Chem. 61:7358-73, which are herein incorporated by reference in their entireties.

Non-limiting examples of targeting moieties are described below.

i. Ligands

A ligand functions as a type of targeting moiety defined as a selectively bindable material that has a selective (or specific), affinity for another substance. The ligand is recognized and bound by a usually, but not necessarily, larger specific binding body or “binding partner,” or “receptor.” Examples of ligands suitable for targeting are antigens, haptens, biotin, biotin derivatives, lectins, galactosamine and fucosylamine moieties, receptors, substrates, coenzymes and cofactors among others.

When applied to the micelles of the present disclosure a ligand includes an antigen or hapten that is capable of being bound by, or to, its corresponding antibody or fraction thereof. Also included are viral antigens or hemagglutinins and neuraminidases and nucleocapsids including those from any DNA and RNA viruses, AIDS, HIV and hepatitis viruses, adenoviruses, alphaviruses, arenaviruses, coronaviruses, flaviviruses, herpesviruses, myxoviruses, oncornaviruses, papovaviruses, paramyxoviruses, parvoviruses, picornaviruses, poxviruses, reoviruses, rhabdoviruses, rhinoviruses, togaviruses and viroids; any bacterial antigens including those of gram-negative and gram-positive bacteria, Acinetobacter, Achromobacter, Bacteroides, Clostridium, Chlamydia, enterobacteria, Haemophilus, Lactobacillus, Neisseria, Staphyloccus, or Streptoccocus; any fungal antigens including those of Aspergillus, Candida, Coccidiodes, mycoses, phycomycetes, and yeasts; any mycoplasma antigens; any rickettsial antigens; any protozoan antigens; any parasite antigens; any human antigens including those of blood cells, virus infected cells, genetic markers, heart diseases, oncoproteins, plasma proteins, complement factors, rheumatoid factors. Included are cancer and tumor antigens such as alpha-fetoproteins, prostate specific antigen (PSA) and CEA, cancer markers and oncoproteins, among others.

Other substances that can function as ligands for targeting a micelle of the present disclosure are certain vitamins (i.e. folic acid, B12), steroids, prostaglandins, carbohydrates, lipids, antibiotics, drugs, digoxins, pesticides, narcotics, neuro-transmitters, and substances used or modified such that they function as ligands.

In some aspects, the targeting moiety comprises a protein or protein fragment (e.g., hormones, toxins), and synthetic or natural polypeptides with cell affinity. Ligands also include various substances with selective affinity for ligators that are produced through recombinant DNA, genetic and molecular engineering. Except when stated otherwise, ligands of the instant disclosure also include ligands as defined in U.S. Pat. No. 3,817,837, which is herein incorporated by reference in its entirety.

ii. Ligators

A ligator functions as a type of targeting moiety defined for this disclosure as a specific binding body or “partner” or “receptor,” that is usually, but not necessarily, larger than the ligand it can bind to. For the purposes of this disclosure, it can be a specific substance or material or chemical or “reactant” that is capable of selective affinity binding with a specific ligand. A ligator can be a protein such as an antibody, a nonprotein binding body, or a “specific reactor.”

When applied to this disclosure, a ligator includes an antibody, which is defined to include all classes of antibodies, monoclonal antibodies, chimeric antibodies, Fab fractions, fragments and derivatives thereof. The term “antibody” encompasses an immunoglobulin whether natural or partly or wholly synthetically produced, and fragments thereof. The term also covers any protein having a binding domain that is homologous to an immunoglobulin binding domain. “Antibody” further includes a polypeptide comprising a framework region from an immunoglobulin gene or fragments thereof that specifically binds and recognizes an antigen. Use of the term antibody is meant to include whole antibodies, polyclonal, monoclonal and recombinant antibodies, fragments thereof, and further includes single-chain antibodies, humanized antibodies, murine antibodies, chimeric, mouse-human, mouse-primate, primate-human monoclonal antibodies, anti-idiotype antibodies, antibody fragments, such as, e.g., scFv, scFab, (scFab)₂, (scFv)₂, Fab, Fab′, and F(ab′)₂, F(ab1)₂, Fv, dAb, and Fd fragments, diabodies, and antibody-related polypeptides. Antibody includes bispecific antibodies and multispecific antibodies so long as they exhibit the desired biological activity or function. In some aspects of the present disclosure, the targeting moiety is an antibody or a molecule comprising an antigen-binding fragment thereof. In some aspects, the antibody is a nanobody. In some aspects, the antibody is an ADC. The terms “antibody-drug conjugate” and “ADC” are used interchangeably and refer to an antibody linked, e.g., covalently, to a therapeutic agent (sometimes referred to herein as agent, drug, or active pharmaceutical ingredient) or agents. In some aspects of the present disclosure, the targeting moiety is an antibody-drug conjugate.

Under certain conditions, the instant disclosure is also applicable to using other substances as ligators. For instance, other ligators suitable for targeting include naturally occurring receptors, any hemagglutinins and cell membrane and nuclear derivatives that bind specifically to hormones, vitamins, drugs, antibiotics, cancer markers, genetic markers, viruses, and histocompatibility markers. Another group of ligators includes any RNA and DNA binding substances such as polyethylenimine (PEI) and polypeptides or proteins such as histones and protamines.

Other ligators also include enzymes, especially cell surface enzymes such as neuraminidases, plasma proteins, avidins, streptavidins, chalones, cavitands, thyroglobulin, intrinsic factor, globulins, chelators, surfactants, organometallic substances, staphylococcal protein A, protein G, ribosomes, bacteriophages, cytochromes, lectins, certain resins, and organic polymers.

Targeting moieties also include various substances such as any proteins, protein fragments or polypeptides with affinity for the surface of any cells, tissues or microorganisms that are produced through recombinant DNA, genetic and molecular engineering. Thus, in some aspects, the targeting moiety directs a micelle of the present disclosure to a specific tissue (i.e., liver tissue or brain tissue), to a specific type of cell (e.g., a certain type of cancer cells), or to a physiological compartment or physiological barrier (e.g., the BBB).

e. Linkers

As described above, a cationic carrier unit disclosed herein can comprise one or more linkers. As used herein, the term “linker” refers to a peptide or polypeptide sequence (e.g., a synthetic peptide or polypeptide sequence), or a non-peptide linker for which its main function is to connect two moieties in a cationic carrier unit disclosed herein. In some aspects, cationic carrier units of the present disclosure can comprise at least one linker connecting a tissue-specific targeting moiety (TM) with a water-soluble polymer (WS), at least one linker connecting a water-soluble biopolymer (WP) with cationic carrier (CC) or a hydrophobic moiety (HM) or a crosslinking moiety (CM), at least one linker connecting a cationic carrier (CC) with a hydrophobic moiety (HM), or any combination thereof. In some aspects, two or more linkers can be linked in tandem.

When multiple linkers are present in a cationic carrier unit disclosed herein, each of the linkers can be the same or different. Generally, linkers provide flexibility to the cationic carrier unit. Linkers are not typically cleaved; however, in certain aspects, such cleavage can be desirable. Accordingly, in some aspects a linker can comprise one or more protease-cleavable sites, which can be located within the sequence of the linker or flanking the linker at either end of the linker sequence.

In one aspect, the linker is a peptide linker. In some aspects, the peptide linker can comprise at least about two, at least about three, at least about four, at least about five, at least about 10, at least about 15, at least about 20, at least about 25, at least about 30, at least about 35, at least about 40, at least about 45, at least about 50, at least about 55, at least about 60, at least about 65, at least about 70, at least about 75, at least about 80, at least about 85, at least about 90, at least about 95, or at least about 100 amino acids.

In some aspects, the peptide linker can comprise at least about 110, at least about 120, at least about 130, at least about 140, at least about 150, at least about 160, at least about 170, at least about 180, at least about 190, or at least about 200 amino acids.

In other aspects, the peptide linker can comprise at least about 200, at least about 250, at least about 300, at least about 350, at least about 400, at least about 450, at least about 500, at least 550, at least about 600, at least about 650, at least about 700, at least about 750, at least about 800, at least about 850, at least about 900, at least about 950, or at least about 1,000 amino acids.

The peptide linker can comprise between 1 and about 5 amino acids, between 1 and about 10 amino acids, between 1 and about 20 amino acids, between about 10 and about 50 amino acids, between about 50 and about 100 amino acids, between about 100 and about 200 amino acids, between about 200 and about 300 amino acids, between about 300 and about 400 amino acids, between about 400 and about 500 amino acids, between about 500 and about 600 amino acids, between about 600 and about 700 amino acids, between about 700 and about 800 amino acids, between about 800 and about 900 amino acids, or between about 900 and about 1000 amino acids.

Examples of peptide linkers are well known in the art. In some aspects, the linker is a glycine/serine linker. In some aspects, the peptide linker is glycine/serine linker according to the formula [(Gly)ₙ-Ser]ₘ where n is any integer from 1 to 100 and m is any integer from 1 to 100. In other aspects the glycine/serine linker is according to the formula [(Gly)ₓ-Ser_{y}]_{z} (SEQ ID NO: 1) wherein x in an integer from 1 to 4, y is 0 or 1, and z is an integer from 1 to 50. In one aspect, the peptide linker comprises the sequence Gₙ, where n can be an integer from 1 to 100. In a specific aspect, the sequence of the peptide linker is GGGG (SEQ ID NO: 2).

In some aspects, the peptide linker can comprise the sequence (GlyAla)ₙ (SEQ ID NO: 3), wherein n is an integer between 1 and 100. In other aspects, the peptide linker can comprise the sequence (GlyGlySer)ₙ (SEQ ID NO: 4), wherein n is an integer between 1 and 100.

In other aspects, the peptide linker comprises the sequence (GGGS)ₙ (SEQ ID NO: 5). In still other aspects, the peptide linker comprises the sequence (GGS)ₙ(GGGGS)ₙ (SEQ ID NO: 6). In these instances, n can be an integer from 1 to 100. In other instances, n can be an integer from 1 to 20, i.e., 1, 2, 3, 4, 5, 6, 7, 8, 9, 10, 11, 12, 13, 14, 15, 16, 17, 18, 19, or 20.

Examples of linkers include, but are not limited to, GGG, SGGSGGS (SEQ ID NO: 7), GGSGGSGGSGGSGGG (SEQ ID NO: 8), GGSGGSGGGGSGGGGS (SEQ ID NO: 9), GGSGGSGGSGGSGGSGGS (SEQ ID NO: 10), or GGGGSGGGGSGGGGS (SEQ ID NO: 11).

In other aspects, the linker is a poly-G sequence (GGGG)ₙ (SEQ ID NO: 12), where n can be an integer from 1 to 100.

In one aspect, the peptide linker is synthetic, i.e., non-naturally occurring. In one aspect, a peptide linker includes peptides (or polypeptides) (e.g., natural or non-naturally occurring peptides) which comprise an amino acid sequence that links or genetically fuses a first linear sequence of amino acids to a second linear sequence of amino acids to which it is not naturally linked or genetically fused in nature. For example, in one aspect the peptide linker can comprise non-naturally occurring polypeptides that are modified forms of naturally occurring polypeptides (e.g., comprising a mutation such as an addition, substitution or deletion). In another aspect, the peptide linker can comprise non-naturally occurring amino acids. In another aspect, the peptide linker can comprise naturally occurring amino acids occurring in a linear sequence that does not occur in nature. In still another aspect, the peptide linker can comprise a naturally occurring polypeptide sequence.

In some aspects, the linker comprises a non-peptide linker. In other aspects, the linker consists of a non-peptide linker. In some aspects, the non-peptide linker can comprise, e.g., maleimido caproyl (MC), maleimido propanoyl (MP), methoxyl polyethyleneglycol (MPEG), succinimidyl 4-(N-maleimidomethyl)-cyclohexane-1-carboxylate (SMCC), m-maleimidobenzoyl-N-hydroxysuccinimide ester (MBS), succinimidyl 4-(p-maleimidophenyl) butyrate (SMPB), N-succinimidyl (4-iodoacetyl)aminobenzonate (SIAB), succinimidyl 6-[3-(2-pyridyldithio)-propionamide] hexanoate (LC-SPDP), 4-succinimidyloxycarbonyl-alpha-methyl-alpha-(2-pyridyldithio) toluene (SMPT), etc., including residues thereof (see, e.g., U.S. Pat. No. 7,375,078).

Linkers can be introduced into polypeptide sequences using techniques known in the art (e.g., chemical conjugation, recombinant techniques, or peptide synthesis). Modifications can be confirmed by DNA sequence analysis. In some aspects, the linkers can be introduced using recombinant techniques. In other aspects, the linkers can be introduced using solid phase peptide synthesis. In certain aspects, a cationic carrier unit disclosed herein can contain simultaneously one or more linkers that have been introduced using recombinant techniques and one or more linkers that have been introduced using solid phase peptide synthesis or methods of chemical conjugation known in the art. In some aspects, the linker comprises a cleavage site.

### III. Payloads

As used herein the term “payload” refers to a biologically active molecule, e.g., a therapeutic agent that can interactive by itself or via an adapter with a cationic carrier unit of the present disclosure, and be included within the core of a micelle of the present disclosure.

Other biologically active molecules are anti-viral drugs, nucleic acids and other anti-viral substances including those against any DNA and RNA viruses, acquired immunodeficiency syndrome (AIDS), human immunodeficiency virus (HIV), hepatitis viruses, adenoviruses, alphaviruses, arenaviruses, coronaviruses, flaviviruses, herpesviruses, myxoviruses, oncornaviruses, papovaviruses, paramyxoviruses, parvoviruses, picomaviruses, poxviruses, reoviruses, thabdoviruses, rhinoviruses, togaviruses and viriods; any anti-bacterial drugs, nucleic acids and other anti-bacterial substances including those against gram-negative and grampositive bacteria, Acinetobacter, Achromobacter, Bacteroides, Clostridium, Chlamydia, enterobacteria, Haemophilus, Lactobacillus, Neisseria, Staphyloccus, or Streptoccocus; any antifungal drugs, nucleic acids and other anti-fungal substances including those against Aspergillus, Candida, Coccidiodes, mycoses, phycomycetes, and yeasts; any drugs, nucleic acids and other substances against mycoplasma and rickettsia; any anti-protozoan drugs, nucleic acids and other substances; any anti-parasitic drugs, nucleic acids and other substances; any drugs, nucleic acids and other substances against heart diseases, tumors, and virus infected cells, among others.

### (a) Nucleic Acids

In some aspects, the biologically active molecule (payload) is a nucleic acid, e.g., an RNA or a DNA. Nucleic acid active agents suitable for delivery using the micelles of the present disclosure include all types of RNA and all types of DNA, including also oligonucleotides such as probes and primers used in the polymerase chain reaction (PCR), hybridizations, or DNA sequencing. In some aspects, the nucleic acid comprises mRNA, miRNA, miRNA sponge, tough decoy miRNA (TD), antimir (antagomir), small RNA, IRNA, siRNA, shRNA, gDNA, cDNA, pDNA, PNA, BNA, antisense oligonucleotide (ASO), aptamer, cyclic dinucleotide, or any combination thereof.

In some aspects, the biologically active molecule (payload) comprises a short interfering RNA (siRNA), which is a double-stranded RNA that can induce sequence-specific post-transcriptional gene silencing, thereby decreasing or even inhibiting gene expression. For example, siRNAs can trigger the specific degradation of homologous RNA molecules, such as mRNAs, within the region of sequence identity between both the siRNA and the target RNA. Non-limiting exemplary siRNAs are disclosed in WO 02/44321, which is incorporated by reference in its entirety. In some aspects, siRNA can be about 20-27 base pairs in length.

In some aspects, the siRNA can be chemically modified. In some aspects, the siRNA can be conjugated to cholesterol. In some aspects, the cholesterol can be conjugated to a 3′ end of a sense or antisense strand of the siRNA. In some aspects, the cholesterol can be conjugated to a 5′ end of a sense or antisense strand of the siRNA. In some aspects, the cholesterol can be conjugated to both the 3′ and 5′ end of a sense or antisense strand of the siRNA.

As used herein, the number of charge in siRNA is the number of nucleotides-2.

In some aspects, the biologically active molecule (e.g., anionic payload) comprises a short hairpin RNAs (shRNAs). In some aspects, the biologically active molecule comprises an miRNA or a miRNA inhibitor (antimiR). In some aspects, the biologically active molecule (e.g., anionic payload) can be 10-30 nucleotides in length, for example from 14-25 nucleotides in length. In some aspects, the biologically active molecule (e.g. anionic payload) has a length of 16-30 nucleotides, 18-25 nucleotides, particularly 18, 19, 20, 21, 22, 23, 24, or 25 nucleotides. In some aspects, the biologically active molecule (e.g., anionic payload) comprises a nucleotide sequence having less than 200 nucleotides in length. In some aspects, the anionic payload comprises a nucleotide sequence having less than about 150, about 140, about 130, about 120, about 110, about 100, about 90, about 80, about 70, about 60, about 50, about 40, about 30, about 25, about 24, about 23, about 22, about 21, about 20, about 19, about 18, about 17, about 16, about 15, about 14, about 13, about 12, about 11, or about 10 nucleotides in length. In some aspects, the anionic payload comprises a nucleotide sequence having from about 30 to about 10, from about 25 to about 11, from about 30 to about 15, from about 25 to about 15, from about 24 to about 15, or from about 23 to about 15 nucleotides in length. In some aspects, the anionic payload comprises a nucleotide sequence having about 30, about 29, about 28, about 27, about 26, about 25, about 24, about 23, about 22, about 21, about 20, about 19, about 18, about 17, about 16, about 15, about 14, or about 13 nucleotides in length. In some aspects, the anionic payload comprises a nucleotide sequence having about 22 nucleotides in length.

Sequences for miRNAs are available publicly, for example, through the miRBase registry (Griffiths-Jones, et al., Nucleic Acids Res., 36 (Database Issue): D154-D158 (2008); Griffiths-Jones, et al., Nucleic Acids Res., 36 (Database Issue): D140-D144 (2008); Griffiths-Jones, et al., Nucleic Acids Res., 36 (Database Issue): D109-D111 (2008)) and other publically accessible databases.

In some aspects, the miRNA inhibitors are oligomers or polymers of ribonucleic acid (RNA) or deoxyribonucleic acid (DNA) or modifications thereof. In some aspects, the miRNA antagonists are antimir. Antimirs are a specific class of miRNA inhibitors that are described, for example, in U.S. Patent Application Publication 2007/0213292. Antimirs are RNA-like oligonucleotides that contain various modifications for RNase protection and pharmacologic properties such as enhanced tissue and cellular uptake. Antimirs differ from normal RNA by having complete 2′—O-methylation of sugar, a phosphorothioate backbone, and a cholesterol-moiety at 3′-end.

Non-limiting examples of antimirs and other miRNA inhibitors are described in WO2009/020771, WO2008/091703, WO2008/046911, WO2008/074328, WO2007/090073, WO2007/027775, WO2007/027894, WO2007/021896, WO2006/093526, WO2006/112872, WO2007/112753, WO2007/112754, WO2005/023986, or WO2005/013901, all of which are hereby incorporated by reference.

In some aspects, the anionic paylod comprises mRNA, miRNA, miRNA sponge, tough decoy miRNA, antimir, small RNA, rRNA, siRNA, shRNA, gDNA, cDNA, pDNA, PNA, BNA, antisense oligonucleotide (ASO), aptamer, cyclic dinucleotide, or any combination thereof. In some aspects, the anionic payload is an siRNA. In some aspects, the anionic payload is mRNA. In some aspects, the anionic payload is a PNA.

In some aspects, the nucleic acids are phosphodiester antisense oligonucleotides, and any oligonucleotides where the sugar-phosphate “backbone” has been derivatized or replaced with “backbone analogues” such as with phosphorothioate, phosphorodithioate, phosphoroamidate, alkyl phosphotriester, or methylphosphonate linkages. In some aspects, the nucleic acids active agents are antisense oligonucleotides, and any oligonucleotides or oligodeoxynucleotides with non-phosphorous backbone analogues such as sulfamate, 3′-thioformacetal, methylene(methylimino) (MMI), 3′-N-carbamate, or morpholino carbamate.

In some aspects, the biologically active molecule (payload) is an antimir. As used herein, the terms “antimir,” “anti microRNA,” “anti miRNA,” and variants thereof refer to molecules (e.g., synthetically generated molecules) that are used to neutralize microRNA (miRNA) function in cells for desired responses. miRNA are complementary sequences (approx. 20-22 base pairs (bp)) to mRNA that are involved in the cleavage of RNA or the suppression of the translation. By controlling the miRNA that regulate mRNAs in cells, antimirs (also called anti-miRNA oligonucleotides, AMOs, or antagomirs) can be used as further regulation as well as for therapeutic for certain cellular disorders. This regulation can occur through a steric blocking mechanism as well as hybridization to miRNA.

These interactions within the body between antimirs and a miRNA can be for therapeutics in disorders in which over/under expression occurs or aberrations in miRNA lead to coding issues. Some of the miRNA linked disorders that are encountered in the humans include cancers, muscular diseases, autoimmune disorders, and viruses.

Various components of antimirs can be manipulated to affect the binding affinity and potency of the antimir. The 2′-sugar of the antimirs can be modified to be substituted with fluorine and various methyl groups, almost all with an increase in binding affinity. However, some of these modified 2′-sugar antimirs lead to negative effects on cell growth. Modifying the 5′-3′ phosphodiester backbone linkage to a phosphorothioate (P-S) backbone linkage is also known to have an effect on target affinity. Using the P-S mutation was shown to decrease the Tm of the oligonucleotide, which leads to a lower target affinity. A final requirement for antimirs is mismatch specificity and length restrictions. Due to miRNAs in the same families sharing “seed” (shared) sequences and differ by only a couple of additional nucleotides; one antimir can potentially target multiple miRNA sequences. One or more examples of antimirs or miRNA sequences are shown in the following table.

TABLE 1

| SEQ ID | | | | SEQ ID | Artificial miRNA |
|---|---|---|---|---|---|
| NO for | Target | miRNA | | NO for | inhibitor |
| miRNA | Score | Name | Mature miRNA sequence | antimir | sequence (antimir) |
| 13 | 95 | hsa-miR- | UUCCCUUUGUCAUCCUAUGCCU | 15 | AGGCAUAGGAUGACAAAGGGAA |
| | | 204-5p | | | |
| 14 | 89 | hsa-miR- | UAACAGUCUACAGCCAUGGUCG | 16 | CGACCAUGGCUGUAGACUGUUA |
| | | 132-3p | | | |

In some aspects, the payload is a polynucleotide comprising a nucleotide sequence having 5 to 30 nucleotides in length. In some aspects, the polynucleotide has 5, 6, 7, 8, 9, 10, 11, 12, 13, 14, 15, 16, 17, 18, 19, 20, 21, 22, 23, 24, 25, 26, 27, 28, 29, or 30 nucleotides in length. In some aspects, the nucleotide sequence has 15, 16, 17, 18, 19, 20, 21, 22, 23, 24, 25, or 26 nucleotides in length. In some aspects, the payload comprises a nucleotide sequence having less than 200 nucleotides in length. In some aspects, the payload comprises a nucleotide sequence having less than about 150, about 140, about 130, about 120, about 110, about 100, about 90, about 80, about 70, about 60, about 50, about 40, about 30, about 25, about 24, about 23, about 22, about 21, about 20, about 19, about 18, about 17, about 16, about 15, about 14, about 13, about 12, about 11, or about 10 nucleotides in length. In some aspects, the payload comprises a nucleotide sequence having from about 30 to about 10, from about 25 to about 11, from about 30 to about 15, from about 25 to about 15, from about 24 to about 15, or from about 23 to about 15 nucleotides in length. In some aspects, the payload comprises a nucleotide sequence having about 30, about 29, about 28, about 27, about 26, about 25, about 24, about 23, about 22, about 21, about 20, about 19, about 18, about 17, about 16, about 15, about 14, or about 13 nucleotides in length.

In some aspects, the payload (e.g., antimir) is a nucleotide sequence targeting hsa-miR-485, e.g., hsa-miR-485-3p. In some aspects, the hsa-miR-485-3p has the sequence GUCAUACACGGCUCUCCUCUCU (SEQ ID NO: 17). In some aspects, the payload (e.g., antimir) is a nucleotide sequence comprising, consisting essentially of, or consisting of AGAGAGGAGAGCCGUGUAUGAC (SEQ ID NO: 18), wherein U can be optionally T. In some aspects, the payload (e.g., antimir) is a nucleotide sequence comprising, consisting essentially of, or consisting of AGAGAGGAGAGCCGUGUAUGAC (SEQ ID NO: 18), wherein the nucleotide sequence has one mismatch, two mismatches, three mismatches, or four mismatches. In some aspects, the payload (e.g., antimir) is a nucleotide sequence comprising, consisting essentially of, or consisting of AGAGAGGAGAGCCGUGUAUGAC (SEQ ID NO: 18), wherein the nucleotide sequence has one or two mismatches. In other aspects, the payload (e.g., antimir) is a nucleotide sequence targeting the seed sequence of hsa-miR-485-3p (UCAUACA; SEQ ID NO: 19). In some aspects, the payload (e.g., antimir) is a nucleotide sequence comprising UCAUACA (SEQ ID NO: 19), wherein U can be optionally T (complement of the seed), wherein the nucleotide sequence is about 10 nucleotides to 30 nucleotides (e.g., 10 to 25, 10 to 24, 10 to 23, 10 to 22, 10 to 21, 10 to 20, 10 to 19, or 10 to 18) in length. In some aspects, the payload (e.g., antimir) is a nucleotide sequence comprising UGUAUGA (SEQ ID NO: 20), wherein U can be optionally T (complement of the seed), wherein the nucleotide sequence comprises one, two three, four, five, six, seven, eight, nine, ten, 11, 12, 13, 14, 15, 16, 17, 18, 19, or 20 nucleic acids at the 5′ terminus of the complement of the seed sequence and/or one, two three, four, five, six, seven, eight, nine, ten, 11, 12, 13, 14, 15, 16, 17, 18, 19, or 20 nucleic acids at the 3′ terminus of the complement of the seed sequence.

In some aspects, the payload is a nucleotide sequence selected from the group consisting of: 5′-UGUAUGA-3′ (SEQ ID NO: 23), 5′-GUGUAUGA-3′ (SEQ ID NO: 24), 5′-CGUGUAUGA-3′ (SEQ ID NO: 25), 5′-CCGUGUAUGA-3′ (SEQ ID NO: 26), 5′-GCCGUGUAUGA-3′ (SEQ ID NO: 27), 5′-AGCCGUGUAUGA-3′ (SEQ ID NO: 28), 5′-GAGCCGUGUAUGA-3′ (SEQ ID NO: 29), 5′-AGAGCCGUGUAUGA-3′ (SEQ ID NO: 30), 5′-GAGAGCCGUGUAUGA-3′ (SEQ ID NO: 31), 5′-GGAGAGCCGUGUAUGA-3′ (SEQ ID NO: 32), 5′-AGGAGAGCCGUGUAUGA-3′ (SEQ ID NO: 33), 5′-GAGGAGAGCCGUGUAUGA-3′ (SEQ ID NO: 34), 5′-AGAGGAGAGCCGUGUAUGA-3′ (SEQ ID NO: 35), 5′-GAGAGGAGAGCCGUGUAUGA-3′ (SEQ ID NO: 36); 5′-UGUAUGAC-3′ (SEQ ID NO: 37), 5′-GUGUAUGAC-3′ (SEQ ID NO: 38), 5′-CGUGUAUGAC-3′ (SEQ ID NO: 39), 5′-CCGUGUAUGAC-3′ (SEQ ID NO: 40), 5′-GCCGUGUAUGAC-3′ (SEQ ID NO: 41), 5′-AGCCGUGUAUGAC-3′ (SEQ ID NO: 42), 5′-GAGCCGUGUAUGAC-3′ (SEQ ID NO: 43), 5′-AGAGCCGUGUAUGAC-3′ (SEQ ID NO: 44), 5′-GAGAGCCGUGUAUGAC-3′ (SEQ ID NO: 45), 5′-GGAGAGCCGUGUAUGAC-3′ (SEQ ID NO: 46), 5′-AGGAGAGCCGUGUAUGAC-3′(SEQ ID NO: 47), 5′-GAGGAGAGCCGUGUAUGAC-3′ (SEQ ID NO: 48), 5′-AGAGGAGAGCCGUGUAUGAC-3′ NO: (SEQ ID 49), or 5′-GAGAGGAGAGCCGUGUAUGAC-3′ (SEQ ID NO: 50).

In some aspects, the payload is a nucleotide sequence comprising 5′-TGTATGA-3′ (SEQ ID NO: 51), 5′-GTGTATGA-3′ (SEQ ID NO: 52), 5′-CGTGTATGA-3′ (SEQ ID NO: 53), 5′-CCGTGTATGA-3′ (SEQ ID NO: 54), 5′-GCCGTGTATGA-3′ (SEQ ID NO: 55), 5′-AGCCGTGTATGA-3′ (SEQ ID NO: 56), 5′-GAGCCGTGTATGA-3′ (SEQ ID NO: 57), 5′-AGAGCCGTGTATGA-3′ (SEQ ID NO: 58), 5′-GAGAGCCGTGTATGA-3′ (SEQ ID NO: 59), 5′-GGAGAGCCGTGTATGA-3′ (SEQ ID NO: 60), 5′-AGGAGAGCCGTGTATGA-3′ (SEQ ID NO: 61), 5′-GAGGAGAGCCGTGTATGA-3′ (SEQ ID NO: 62), 5′-AGAGGAGAGCCGTGTATGA-3′ (SEQ ID NO: 63), 5′-GAGAGGAGAGCCGTGTATGA-3′ (SEQ ID NO: 64); 5′-TGTATGAC-3′ (SEQ ID NO: 65), 5′-GTGTATGAC-3′ (SEQ ID NO: 66), 5′-CGTGTATGAC-3′ (SEQ ID NO: 67), 5′-CCGTGTATGAC-3′ (SEQ ID NO: 68), 5′-GCCGTGTATGAC-3′ (SEQ ID NO: 69), 5′-AGCCGTGTATGAC-3′ (SEQ ID NO: 70), 5′-GAGCCGTGTATGAC-3′ (SEQ ID NO: 71), 5′-AGAGCCGTGTATGAC-3′ (SEQ ID NO: 72), 5′-GAGAGCCGTGTATGAC-3′ (SEQ ID NO: 73), 5′-GGAGAGCCGTGTATGAC-3′ (SEQ ID NO: 74), 5′-AGGAGAGCCGTGTATGAC-3′ (SEQ ID NO: 75), 5′-GAGGAGAGCCGTGTATGAC-3′ (SEQ ID NO: 76), 5′-AGAGGAGAGCCGTGTATGAC-3′ (SEQ ID NO: 77), or 5′-GAGAGGAGAGCCGTGTATGAC-3′ (SEQ ID NO: 78).

In some aspects, the payload (e.g., antimir) is a nucleotide sequence targeting hsa-miR-204, e.g., hsa-miR-204-5p. The hsa-miR-204-5p is shown at TABLE 1 as UUCCCUUUGUCAUCCUAUGCCU (SEQ ID NO: 13). In some aspects, the payload (e.g., antimir) is a nucleotide sequence comprising, consisting essentially of, or consisting of AGGCAUAGGAUGACAAAGGGAA (SEQ ID NO: 15), wherein U can be optionally T. In some aspects, the payload (e.g., antimir) is a nucleotide sequence comprising, consisting essentially of, or consisting of AGGCAUAGGAUGACAAAGGGAA (SEQ ID NO: 15), wherein U can be optionally T and wherein the nucleotide sequence has one mismatch, two mismatches, three mismatches, or four mismatches. In some aspects, the payload (e.g., antimir) is a nucleotide sequence comprising, consisting essentially of, or consisting of AGGCAUAGGAUGACAAAGGGAA (SEQ ID NO: 15), wherein U can be optionally T and wherein the nucleotide sequence has one or two mismatches. In other aspects, the payload (e.g., antimir) is a nucleotide sequence targeting the seed sequence of hsa-miR-204-5p (UCCCUUU; SEQ ID NO: 21). In some aspects, the payload (e.g., antimir) is a nucleotide sequence comprising AAAGGGA (SEQ ID NO: 22) (complement of the seed), wherein U can be optionally T and wherein the nucleotide sequence is about 10 nucleotides to 30 nucleotides (e.g., 10 to 25, 10 to 24, 10 to 23, 10 to 22, 10 to 21, 10 to 20, 10 to 19, or 10 to 18) in length. In some aspects, the payload (e.g., antimir) is a nucleotide sequence comprising AAAGGGA (SEQ ID NO: 22) (complement to the seed), wherein the nucleotide sequence comprises one, two three, four, five, six, seven, eight, nine, ten, 11, 12, 13, 14, 15, 16, 17, 18, 19, or 20 nucleic acids at the 5′ terminus of the complement of the seed sequence and/or one, two three, four, five, six, seven, eight, nine, ten, 11, 12, 13, 14, 15, 16, 17, 18, 19, or 20 nucleic acids at the 3′ terminus of the complement of the seed sequence.

i. Chemically Modified Polynucleotides

In some aspects, a polynucleotide of the present disclosure (e.g., an antimir, e.g., an miR485 antimir) comprises at least one chemically modified nucleoside and/or nucleotide. When the polynucleotides of the present disclosure are chemically modified, the polynucleotides can be referred to as “modified polynucleotides.”

A “nucleoside” refers to a compound containing a sugar molecule (e.g., a pentose or ribose) or a derivative thereof in combination with an organic base (e.g., a purine or pyrimidine) or a derivative thereof (also referred to herein as “nucleobase”).

A “nucleotide” refers to a nucleoside including a phosphate group. Modified nucleotides can be synthesized by any useful method, such as, for example, chemically, enzymatically, or recombinantly, to include one or more modified or non-natural nucleosides.

Polynucleotides can comprise a region or regions of linked nucleosides. Such regions can have variable backbone linkages. The linkages can be standard phosphodiester linkages, in which case the polynucleotides would comprise regions of nucleotides.

The modified polynucleotides disclosed herein can comprise various distinct modifications. In some aspects, the modified polynucleotides contain one, two, or more (optionally different) nucleoside or nucleotide modifications. In some aspects, a modified polynucleotide can exhibit one or more desirable properties, e.g., improved thermal or chemical stability, reduced immunogenicity, reduced degradation, increased binding to the target microRNA, reduced non-specific binding to other microRNA or other molecules, as compared to an unmodified polynucleotide.

In some aspects, a polynucleotide of the present disclosure is chemically modified. As used herein in reference to a polynucleotide, the terms “chemical modification” or, as appropriate, “chemically modified” refer to modification with respect to adenosine (A), guanosine (G), uridine (U), thymidine (T) or cytidine (C) ribo- or deoxyribonucleosides in one or more of their position, pattern, percent or population, including, but not limited to, its nucleobase, sugar, backbone, or any combination thereof.

In some aspects, a polynucleotide of the present disclosure (e.g., an antimir) can have a uniform chemical modification of all or any of the same nucleoside type or a population of modifications produced by downward titration of the same starting modification in all or any of the same nucleoside type, or a measured percent of a chemical modification of all any of the same nucleoside type but with random incorporation In another aspect, the polynucleotide of the present disclosure (e.g., an antimir) can have a uniform chemical modification of two, three, or four of the same nucleoside type throughout the entire polynucleotide (such as all uridines and/or all cytidines, etc. are modified in the same way).

Modified nucleotide base pairing encompasses not only the standard adenine-thymine, adenine-uracil, or guanine-cytosine base pairs, but also base pairs formed between nucleotides and/or modified nucleotides comprising non-standard or modified bases, wherein the arrangement of hydrogen bond donors and hydrogen bond acceptors permits hydrogen bonding between a non-standard base and a standard base or between two complementary non-standard base structures. One example of such non-standard base pairing is the base pairing between the modified nucleobase inosine and adenine, cytosine, or uracil. Any combination of base/sugar or linker can be incorporated into polynucleotides of the present disclosure.

The skilled artisan will appreciate that, except where otherwise noted, polynucleotide sequences set forth in the instant application will recite “T”s in a representative DNA sequence but where the sequence represents RNA, the “T”s would be substituted for “U”s. For example, TD's of the present disclosure can be administered as RNAs, as DNAs, or as hybrid molecules comprising both RNA and DNA units.

In some aspects, the polynucleotide (e.g., an antimir, e.g., an miR485 antimir) includes a combination of at least two (e.g., 2, 3, 4, 5, 6, 7, 8, 8, 10, 11, 12, 13, 14, 15, 16, 17, 18, 18, 20 or more) modified nucleobases.

In some aspects, the nucleobases, sugar, backbone linkages, or any combination thereof in a polynucleotide (e.g., an antimir, e.g., an miR485 antimir) are modified by at least about 5%, at least 10%, at least 15%, at least 20%, at least 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99% or 100%.

### 1. Base Modifications

In certain aspects, the chemical modification is at nucleobases in a polynucleotide of the present disclosure (e.g., an antimir, e.g., an miR485 antimir). In some aspects, the at least one chemically modified nucleoside is a modified uridine (e.g., pseudouridine (ψ), 2-thiouridine (s2U), 1-methyl-pseudouridine (m1ψ), 1-ethyl-pseudouridine (e1ψ), or 5-methoxyuridine (mo5U)), a modified cytosine (e.g., 5-methyl-cytidine (m5C)) a modified adenosine (e.g, 1-methyl-adenosine (m1A), N6-methyl-adenosine (m6A), or 2-methyl-adenine (m2A)), a modified guanosine (e.g., 7-methyl-guanosine (m7G) or 1-methyl-guanosine (m1G)), or a combination thereof.

In some aspects, the polynucleotide of the present disclosure (e.g., an antimir, e.g., an miR485 antimir) is uniformly modified (e.g., fully modified, modified throughout the entire sequence) for a particular modification. For example, a polynucleotide can be uniformly modified with the same type of base modification, e.g., 5-methyl-cytidine (m5C), meaning that all cytosine residues in the polynucleotide sequence are replaced with 5-methyl-cytidine (m5C). Similarly, a polynucleotide can be uniformly modified for any type of nucleoside residue present in the sequence by replacement with a modified nucleoside such as any of those set forth above.

In some aspects, the polynucleotide of the present disclosure (e.g., an antimir, e.g., an miR485 antimir) includes a combination of at least two (e.g., 2, 3, 4 or more) of modified nucleobases. In some aspects, at least about 5%, at least 10%, at least 15%, at least 20%, at least 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99% or 100% of a type of nucleobases in a polynucleotide of the present disclosure (e.g., an antimir, e.g., an miR485 antimir) are modified nucleobases.

### 2. Backbone Modifications

In some aspects, the payload can comprise a “polynucleotide of the present disclosure” (for example comprising an antimir, e.g., an miR485 antimir), wherein the polynucleotide includes any useful modification to the linkages between the nucleosides. Such linkages, including backbone modifications, that are useful in the composition of the present disclosure include, but are not limited to the following: 3′-alkylene phosphonates, 3′-amino phosphoramidate, alkene containing backbones, aminoalkylphosphoramidates, aminoalkylphosphotriesters, boranophosphates, —CH₂—O—N(CH₃)—CH₂—, —CH₂—N(CH₃)—N(CH₃)—CH₂—, —CH₂—NH—CH₂—, chiral phosphonates, chiral phosphorothioates, formacetyl and thioformacetyl backbones, methylene(methylimino), methylene formacetyl and thioformacetyl backbones, methyleneimino and methylenehydrazino backbones, morpholino linkages, —N(CH₃)—CH₂—CH₂—, oligonucleosides with heteroatom internucleoside linkage, phosphinates, phosphoramidates, phosphorodithioates, phosphorothioate internucleoside linkages, phosphorothioates, phosphotriesters, PNA, siloxane backbones, sulfamate backbones, sulfide sulfoxide and sulfone backbones, sulfonate and sulfonamide backbones, thionoalkylphosphonates, thionoalkylphosphotriesters, and thionophosphoramidates.

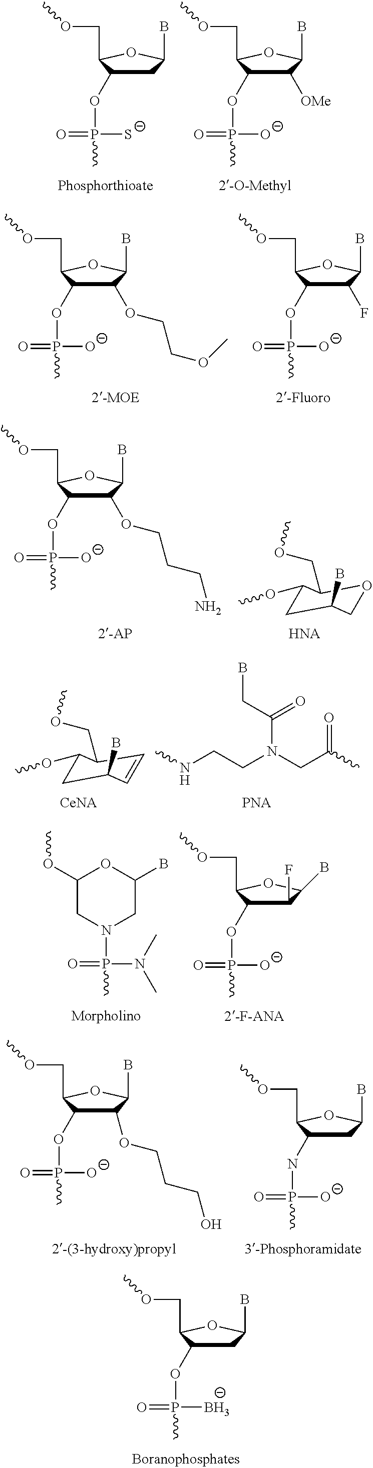

In some aspects, the presence of a backbone linkage disclosed above increase the stability (e.g., thermal stability) and/or resistance to degradation (e.g., enzyme degradation) of a polynucleotide of the present disclosure (e.g., an antimir, e.g., an miR485 antimir). In some aspects, the stability and/or resistance to degradation increases by at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 100% in the modified polynucleotide compared to a corresponding polynucleotide without the modification (reference or control polynucleotide)

In some aspects, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99% or 100% of the backbone linkages in a polynucleotide of the present disclosure ((e.g., an antimir, e.g., an miR485 antimir) are modified (e.g., all of them are phosphorothioate).

In some aspects, 1, 2, 3, 4, 5, 6, 7, 8, 9, 10, 11, 12, 13, 14, 15, 16, 17, 18, 19, 20, or 21 backbone linkages in a polynucleotide of the present disclosure (e.g., an antimir, e.g., an miR 485 antimir) are modified (e.g., phosphorothioate).

In some aspects, the backbone comprises linkages selected from the group consisting of phosphodiester linkage, phosphotriesters linkage, methylphosphonate linkage, phosphoramidate linkage, phosphorothioate linkage, and combinations thereof.

### 3. Sugar Modifications

The modified nucleosides and nucleotides that can be incorporated into a polynucleotide of the present disclosure (e.g., an antimir, e.g., an miR485 antimir), can be modified on the sugar of the nucleic acid. Thus, in some aspects, the payload comprises a nucleic acid, wherein the nucleic comprises at least one nucleoside analog (e.g., a nucleoside with a sugar modification).

In some aspects, the sugar modification increases the affinity of the binding of a polynucleotide to its target miRNA. Incorporating affinity-enhancing nucleotide analogues in the polynucleotide, such as LNA or 2′-substituted sugars can allow the length of polynucleotide to be reduced, and also may reduce the upper limit of the size a polynucleotide before non-specific or aberrant binding takes place.

In some aspects, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or 100% of the nucleotides in a polynucleotide of the present disclosure (e.g., an antimir, e.g., an miR485 antimir) contain sugar modifications (e.g., LNA).

In some aspects, 1, 2, 3, 4, 5, 6, 7, 8, 9, 10, 11, 12, 13, 14, 15, 16, 17, 18, 19, 20, 21, or 22 nucleotide units in a polynucleotide of the present disclosure (e.g., an antimir, e.g., an miR485 antimir) are sugar modified (e.g., LNA).

Generally, RNA includes the sugar group ribose, which is a 5-membered ring having an oxygen. Exemplary, non-limiting modified nucleotides include replacement of the oxygen in ribose (e.g., with S, Se, or alkylene, such as methylene or ethylene); addition of a double bond (e.g., to replace ribose with cyclopentenyl or cyclohexenyl); ring contraction of ribose (e.g., to form a 4-membered ring of cyclobutane or oxetane); ring expansion of ribose (e.g., to form a 6- or 7-membered ring having an additional carbon or heteroatom, such as for anhydrohexitol, altritol, mannitol, cyclohexanyl, cyclohexenyl, and morpholino that also has a phosphoramidate backbone); multicyclic forms (e.g., tricyclo; and “unlocked” forms, such as glycol nucleic acid (GNA) (e.g., R-GNA or S-GNA, where ribose is replaced by glycol units attached to phosphodiester bonds), threose nucleic acid (TNA, where ribose is replace with α-L-threofuranosyl-(3′→2′)), and peptide nucleic acid (PNA, where 2-amino-ethyl-glycine linkages replace the ribose and phosphodiester backbone). The sugar group can also contain one or more carbons that possess the opposite stereochemical configuration than that of the corresponding carbon in ribose. Thus, a polynucleotide molecule can include nucleotides containing, e.g., arabinose, as the sugar.

The 2′ hydroxyl group (OH) of ribose can be modified or replaced with a number of different substituents. Exemplary substitutions at the 2′-position include, but are not limited to, H, halo, optionally substituted C₁₋₆ alkyl; optionally substituted C₁₋₆ alkoxy; optionally substituted C₆₋₁₀ aryloxy; optionally substituted C₃₋₈ cycloalkyl; optionally substituted C₃₋₈ cycloalkoxy; optionally substituted C₆₋₁₀ aryloxy; optionally substituted C₆₋₁₀ aryl-C₁₋₆ alkoxy, optionally substituted C₁₋₁₂ (heterocyclyl)oxy; a sugar (e.g., ribose, pentose, or any described herein); a polyethyleneglycol (PEG), —O(CH₂CH₂O)ₙCH₂CH₂OR, where R is H or optionally substituted alkyl, and n is an integer from 0 to 20 (e.g., from 0 to 4, from 0 to 8, from 0 to 10, from 0 to 16, from 1 to 4, from 1 to 8, from 1 to 10, from 1 to 16, from 1 to 20, from 2 to 4, from 2 to 8, from 2 to 10, from 2 to 16, from 2 to 20, from 4 to 8, from 4 to 10, from 4 to 16, and from 4 to 20); “locked” nucleic acids (LNA) in which the 2′-hydroxyl is connected by a C₁₋₆ alkylene or C₁₋₆ heteroalkylene bridge to the 4′-carbon of the same ribose sugar, where exemplary bridges include methylene, propylene, ether, amino bridges, aminoalkyl, aminoalkoxy, amino, and amino acid.

In some aspects, nucleoside analogues present in a polynucleotide of the present disclosure (e.g., an antimir, e.g., an miR485 antimir) comprise, e.g., 2′—O-alkyl-RNA units, 2′-OMe-RNA units, 2′—O-alkyl-SNA, 2′-amino-DNA units, 2′-fluoro-DNA units, LNA units, arabino nucleic acid (ANA) units, 2′-fluoro-ANA units, HNA units, INA (intercalating nucleic acid) units, 2′-MOE units, or any combination thereof. In some aspects, the LNA is, e.g., oxy-LNA (such as beta-D-oxy-LNA, or alpha-L-oxy-LNA), amino-LNA (such as beta-D-amino-LNA or alpha-L-amino-LNA), thio-LNA (such as beta-D-thio0-LNA or alpha-L-thio-LNA), ENA (such a beta-D-ENA or alpha-L-ENA), or any combination thereof.

In some aspects, nucleoside analogs present in a polynucleotide of the present disclosure comprise Locked Nucleic Acid (LNA); 2′—O-alkyl-RNA; 2′-amino-DNA; 2′-fluoro-DNA; arabino nucleic acid (ANA); 2′-fluoro-ANA, hexitol nucleic acid (HNA), intercalating nucleic acid (INA), constrained ethyl nucleoside (cEt), 2′—O-methyl nucleic acid (2′—OMe), 2′—O-methoxyethyl nucleic acid (2′-MOE), or any combination thereof.

In some aspects, a polynucleotide of the present disclosure (e.g., an antimir, e.g., an miR485 antimir) can comprise both modified RNA nucleotide analogues (e.g., LNA) and DNA units. In some aspects, a polynucleotide of the present disclosure is a gapmer. See, e.g., U.S. Pat. Nos. 8,404,649; 8,580,756; 8,163,708; 9,034,837; all of which are herein incorporated by reference in their entireties. In some aspects, a polynucleotide of the present disclosure is a micromir. See U.S. Patent Application Publication 2018/0201928, which is herein incorporated by reference in its entirety.

### IV. Micelles

The present disclosure also provides micelles comprising the cationic carrier units of the present disclosure. The micelles of the present disclosure comprise cationic carriers unit of the present disclosure and negatively charged payload, wherein the negatively charged payload and the cationic carrier unit are associate with each other. In some aspects, the association is comprises a covalent bond. In other aspects, the association does not comprise a covalent bond. In other aspects, the association is via an ionic bond, i.e., via electrostatic interaction. In some aspects, the negatively charged payload (e.g., a DNA and/or RNA) is not conjugated to the cationic carrier unit by a covalent bond and/or the negatively charged payload interacts with the cationic carrier moiety of the cationic carrier unit only via an ionic interaction.

In some aspects, the cationic carrier units and micelles of the present disclosure protect the payload (e.g., a DNA and/or RNA) from degradation (e.g., by a DNase and/or an RNase). First, the cationic carrier unit is capable of protecting the payload through electrostatic interaction. Secondly, the micelle sequesters the payload to the core of the micelle, i.e., out of the reach of DNases and/or an RNases. In some aspects, the protection of the payload from circulating enzymes (e.g., nucleases) can increase the half-life of the negatively charged payload (e.g., a DNA and/or RNA) compared to the free payload. In some aspects, encapsulation of the payload in a micelle of the present disclosure can increase the plasma half-life of the payload at least about 2-fold, at least about 3-fold, at least about 4-fold, at least about 5-fold, at least about 6-fold, at least about 7-fold, at least about 8-fold, at least about 9-fold, at least about 10-fold, at least about 11-fold, at least about 12-fold, at least about 13-fold, at least about 14-fold, at least about 15-fold, at least about 16-fold, at least about 17-fold, at least about 18-fold, at least about 19-fold, at least about 20-fold, at least about 21-fold, at least about 22-fold, at least about 23-fold, at least about 24-fold, at least about 25-fold, at least about 26-fold, at least about 27-fold, at least about 28-fold, at least about 29-fold, or at least about 30-fold compared to the free payload.

In some aspects, the positive charge of the cationic carrier unit, and in particular the charge of the cationic carrier moiety is sufficient to form a micelle when mixed with a negatively charged payload (e.g., a nucleic acid) in a solution, wherein the overall ionic ratio between the cationic carrier unit, in particular its cationic carrier moiety, and the negatively charged payload (e.g., a nucleic acid) is about 1:1. In some aspects, the positive charge of the cationic carrier unit, and in particular the charge of the cationic carrier moiety is sufficient to form a micelle when mixed with a negatively charged payload (e.g., a nucleic acid) in a solution, wherein the overall ionic ratio between the cationic carrier unit, in particular its cationic carrier moiety, and the negatively charged payload (e.g., a nucleic acid) is about 2:1. In some aspects, the overall ionic ratio between the cationic carrier unit, in particular its cationic carrier moiety, and the negatively charged payload (e.g., a nucleic acid) is higher than 1:1, i.e., an excess of cationic carrier unit is used. In some aspects, the overall ionic ratio between the cationic carrier unit, in particular its cationic carrier moiety, and the negatively charged payload (e.g., a nucleic acid) is lower than 1:1, i.e., an excess of negatively change payload is used.

In some aspects, upon combination with a suitable buffer (e.g., phosphate-buffered saline (PBS)), the complexes formed between the cationic carrier units of the present disclosure and payload (e.g., an antisense oligonucleotides such as an antimir), self-organize to yield micelles.

A micelle is a water-soluble or colloidal structure or aggregate composed of one or more amphiphilic molecules. Amphiphilic molecules are those that contain at least one hydrophilic (polar) moiety and at least one hydrophobic (nonpolar) moiety. “Classic micelles” have a single, central and primarily hydrophobic zone or “core” surrounded by a hydrophilic layer or “shell.” In aqueous solution, the micelle forms an aggregate with the hydrophilic “head” regions of the amphiphilic molecule in contact with the surrounding solvent, sequestering the hydrophobic single-tail regions of the amphiphilic molecule in the micelle core. Micelles are approximately spherical in shape. Other shapes, e.g., ellipsoids, cylinders, rod-like structures, or polymersomes are also possible. The shape and size, and therefore loading capacity, of the micelles disclosed can be modified by altering the ratio between water-soluble biopolymer (e.g., PEG) and cationic carrier (e.g., poly lysine). Depending on the ratio, the carrier units can organize as small particles, small micelles, micelles, rod-like structures, or polymersomes. Thus, the term “micelles of the present disclosure” encompasses not only classic micelles but also small particles, small micelles, micelles, rod-like structures, or polymersomes.

The micelles of the present disclosure can be composed of either a single monomolecular polymer containing hydrophobic and hydrophilic moieties or an aggregate mixture containing many amphiphilic (i.e. surfactant) molecules formed at or above the critical micelle concentration (CMC), in a polar (i.e. aqueous) solution. The micelle is self-assembled from one or more amphiphilic molecules where the moieties are oriented to provide a primarily hydrophobic interior core and a primarily hydrophilic exterior.

Micelles of the present disclosure can range in size from 5 to about 2000 nanometers. In some aspects, the diameter of the micelle is between about 10 nm and about 200 nm. In some aspects, the diameter of the micelle is between about 1 nm and about 100 nm, between about 10 nm and about 100 nm, between about 10 nm and about 90 nm, between about 10 nm and about 80 nm, between about 10 nm and about 70 nm, between about 20 nm and about 100 nm, between about 20 nm and about 90 nm, between about 20 nm and about 80 nm, between about 20 nm and about 70 nm, between about 30 nm and about 100 nm, between about 30 nm and about 90 nm, between about 30 nm and about 80 nm, between about 30 nm and about 70 nm, between about 40 nm and about 100 nm, between about 40 nm and about 90 nm, between about 40 nm and about 80 nm, or between about 40 nm and about 70 nm. In some aspects, the diameter of the micelles of the present disclosure is between about 30 nm and about 60 nm. In some aspects, the diameter of the micelles of the present disclosure is between about 15 nm and about 90 nm. In some aspects, the diameter of the micelles of the present disclosure is between about 15 nm and about 80 nm. In some aspects, the diameter of the micelles of the present disclosure is between about 15 nm and about 70 nm. In some aspects, the diameter of the micelles of the present disclosure is between about 15 nm and about 60 nm. In some aspects, the diameter of the micelles of the present disclosure is between about 15 nm and about 50 nm. In some aspects, the diameter of the micelles of the present disclosure is between about 20 nm and about 60 nm. In some aspects, the diameter of the micelles of the present disclosure is between about 20 nm and about 50 nm. In some aspects, the diameter of the micelles of the present disclosure is between about 20 nm and about 40 nm. In some aspects, the diameter of the micelles of the present disclosure is between about 25 nm and about 35 nm. In some aspects, the diameter of the micelles of the present disclosure is about 32 nm.

In some aspects, the micelle can comprise a single type of antimir, e.g., miR485 antimir. In other aspects, the micelle can comprise more than one type antimir, e.g., (i) antimir with different architectures targeting the same miRNA; (ii) antimir with different architectures targeting different miRNAs; (iii) antimir with the same architecture targeting the same miRNA; or, (iv) combinations thereof.

In some aspects, the micelles of the present disclosure comprise a single type of cationic carrier unit. In other aspects, the micelles of the present disclosure comprise more than one type of cationic carrier unit (e.g., targeting different receptor on the surface of a target cell). In some aspects, micelles of the present disclosure can comprise cationic carrier units with different targeting moieties, different cationic carrier moieties (e.g., to accommodate different payloads), and/or different hydrophobic and/or crosslinking units.

In order to form a micelle with a payload, different types of cationic or anionic carrier unit can be combined together. For example, in order to target blood brain barrier, the micelle of the present disclosure can comprise a cationic (or an anionic) carrier unit linked to a targeting moiety and a cationic (or an anionic) carrier unit not linked to a targeting moiety. In some aspects, a micelle comprises about 50 to about 200 cationic or anionic carrier units. In other aspects, a micelle comprises about 50 to about 150, about 50 to about 140, about 50 to about 130, about 50 to about 120, about 50 to about 110, or about 50 to about 100 cationic or anionic carrier units. In some aspects, a micelle comprises about 60 to about 200 cationic or anionic carrier units. In other aspects, a micelle comprises about 60 to about 150, about 60 to about 140, about 60 to about 130, about 60 to about 120, about 60 to about 110, about 60 to about 100, about 60 to about 90, about 60 to about 80, or about 60 to about 70 cationic or anionic carrier units. In some aspects, a micelle comprises about 70 to about 200 cationic or anionic carrier units. In other aspects, a micelle comprises about 70 to about 150, about 70 to about 140, about 70 to about 130, about 70 to about 120, about 70 to about 110, about 70 to about 100, about 70 to about 90, or about 70 to about 80 cationic or anionic carrier units. In some aspects, a micelle comprises about 80 to about 200 cationic or anionic carrier units. In other aspects, a micelle comprises about 80 to about 150, about 80 to about 140, about 80 to about 130, about 80 to about 120, about 80 to about 110, about 80 to about 100, or about 80 to about 90 cationic or anionic carrier units. In some aspects, a micelle comprises about 90 to about 200 cationic or anionic carrier units. In other aspects, a micelle comprises about 90 to about 150, about 90 to about 140, about 90 to about 130, about 90 to about 120, about 90 to about 110, or about 90 to about 100 cationic or anionic carrier units. In some aspects, a micelle comprises about 100 to about 200 cationic or anionic carrier units. In other aspects, a micelle comprises about 100 to about 150, about 100 to about 140, about 100 to about 130, about 100 to about 120, about 100 to about 110, or about 100 to about 100 cationic or anionic carrier units.

The present disclosure also includes a micelle comprising (i) a nucleotide sequence (e.g., an oligonucleotide about 14, 15, 16, 17, 18, 19, 20, 21, 22, 23, or 24 nucleotides in length) and (ii) a cationic carrier unit described herein. In some aspects, the disclosure is directed to a micelle comprising (i) a nucleotide sequence, e.g., miRNA, or a miRNA inhibitor (e.g., an oligonucleotide about 14, 15, 16, 17, 18, 19, 20, 21, 22, 23, or 24 nucleotides in length), and (ii) about 80 to about 120 (e.g., about 85 to about 115, about 90 to about 110, about 95 to about 105) cationic carrier units described herein, e.g., Schemas I-VI, Schemas I′-VI′, or a combination thereof.

In some aspects, the micelle comprises (i) a nucleotide sequence, e.g., miRNA, or a miRNA inhibitor (e.g., an oligonucleotide about 14, 15, 16, 17, 18, 19, 20, 21, 22, 23, or 24 nucleotides in length), and (ii) about 80 to about 120 (e.g., about 80, about 85, about 90, about 95, about 100, about 105, or about 110) of a cationic carrier unit described herein, e.g., optional [CC]-L1-[CM]-L2-[HM]. In some aspects, the micelle comprises (i) a nucleotide sequence, e.g., miRNA, or a miRNA inhibitor (e.g., an oligonucleotide about 14, 15, 16, 17, 18, 19, 20, 21, 22, 23, or 24 nucleotides in length), and (ii) about 90 to about 110, e.g., about 100, cationic carrier units, wherein (a) about 45 to about 90, e.g., about 80 of the cationic carrier units comprise [CC]-L1-[CM]-L2-[HM] and (b) about 45 to about 55, e.g., about 50 of the cationic carrier units comprise WP-CC-CM, wherein TM is phenyl alanine, WP is (PEG) 5000, and CC is about 40 to about 50 lysines, e.g., about 45, about 46, about 47, about 48, about 49, or about 50 lysines, and wherein each of about 5 to about 15 of lysines, about 10 lysines, is fused to a nicotinic acid derivative.

In some aspects, the disclosure is directed to a micelle comprising (i) a nucleotide sequence, e.g., siRNA (e.g., 21-mer), and (ii) about 35 to about 60 (e.g., about 35, about 40, about 45, about 50, about 55 or about 60) cationic carrier units described herein, wherein the charge ratio between the cationic carrier and the siRNA is about 2.0.

In some aspects, the disclosure is directed to a micelle comprising (i) a nucleotide sequence, e.g., cholesterol conjugated siRNA (e.g., 21-mer), and (ii) about 35 to about 60 (e.g., about 35, about 40, about 45, about 50, about 55 or about 60) cationic carrier units described herein, wherein the charge ratio between the cationic carrier and the siRNA is about 1.0.

In some aspects, a micelle of the present disclosure comprises (i) a nucleotide sequence, e.g., a miR485-3p inhibitor, e.g., 5′-AGAGAGGAGAGCCGUGUAUGAC-3′ (SEQ ID NO: 18), and (ii) about 100 cationic carrier units, wherein (a) about 50 of the cationic carrier units comprise TM-WP-CC-AM and (b) about 50 of the cationic carrier units comprise WP-CC-CM, wherein TM is phenyl alanine, WP is (PEG) 5000, and CC is about 47 lysines, and wherein each of about 10 lysines is fused to a nicotinic acid derivative (nicotinamide).

In some aspects, the micelle can comprise a single payload (e.g., a single oligonucleotide, e.g., an antimir). In other aspects, the micelle can comprise more than one payload (e.g., multiple oligonucleotides, e.g., multiple antimirs).

### V. Methods of Manufacture

The present disclosure also provides methods of making the cationic carrier units and micelles of the present disclosure. In general, the present disclosure provides a method of preparing a cationic carrier unit of the present disclosure comprising synthesizing the cationic carrier unit as described, e.g., in the Examples section. As used herein, the term “synthesizing” refers the assembling the cationic carrier unit using methods known in the art. For example, protein components (e.g., an antibody targeting moiety) can be prepared recombinantly and subsequently conjugated to the other components of the cationic carrier units. In some aspects, each one of the components of the cationic carrier unit can be prepared using methods known in the art, e.g., recombinant protein production, solid phase peptide or nucleic acid synthesis, chemical synthesis, enzymatic synthesis, or any combination thereof, and the resulting component can be conjugated using chemical and/or enzymatic methods known in the art.

The cationic carrier units of the present disclosure can be purified to remove contaminants. In some aspects, the cationic carrier unit comprises a uniform population of cationic carrier units. However, in other aspects, the cationic carrier unit can comprise multiple species (e.g., some of them comprising a targeting moiety, and some comprising the remaining moieties but without a targeting moiety). In some aspects, the manufacture of the cationic carrier units of the present disclosure comprise lyophilization or any other form of dry storage suitable for reconstitution. In some aspects, the preparation of the cationic carrier unit in a dry form takes place after combination of the cationic carrier units with the payload (e.g., a nucleic acid).

In some aspects, the method of preparing a micelle of the present disclosure comprises mixing the cationic carrier unit with the negatively charged payload (e.g., a nucleic acid such an antisense oligonucleotide, e.g., an antimir) at an ionic ratio of 1:1. In some aspects, the cationic carrier unit and the negatively charged payload are combined in solution. In some aspects, after combination of the cationic carrier and the negative charged payload in solution, the resulting solution is lyophilized or dried. In some aspects, the combination of the cationic carrier and the negative charged payload is conducted in dry form.

The ratio of number n of monomer units in the water-soluble polymer (A, e.g., PEG) to the number m of monomer units (e.g., lysines) in the cationic carrier moiety (B, e.g., poly lysine), wherein the number of units n or m in each case can be up to 1,000 units affects the size and shape of the resulting micelles. At mB/(nA+mB) ratios of 0.5, the micelles obtained are classic micelles. If mB/(nA+mB) is above 0.5, the micelles obtained are rod like micelles or polymersomes. If mB/(nA+mB) is below 0.5, the micelles obtained are small micelles or small particles.

The micelles of the present disclosure can be generation using any of the techniques known in the art, for example, vortexing, extrusion, or sonication. The formation of micelles depends on applying conditions that are above the critical micelle concentration (CMC) of a solution comprising the cationic carrier units of the present disclosure. After they reach a certain value of concentration, surfactants begin to associate and to organize themselves into more complex units, such as micelles. The CMC of a solution comprising the cationic carriers of the present disclosure can be determined by any physical property (e.g., surface tension) that shows a distinct transition around the CMC.

The well-known Smith-Ewart theory predicts that the number of particles nucleated leading to the formation of micelles above the CMC is proportional to the surfactant (in the present disclosure, the cationic carrier units complexed or associated to the anionic payload) concentration to the 0.6 power. This is so because for a given surfactant the number of micelles formed generally increases with an increase in the surfactant concentration.

In some aspects, the micelles of the present disclosure can be purified, e.g., to remove contaminants and/or to generate an uniform population of micelles (e.g., micelles having the same size, or micelles having the same payload or the same targeting moiety).

### VI. Pharmaceutical Compositions

The present disclosure also provides pharmaceutical compositions comprising cationic carrier units and/or micelles of the present disclosure (i.e., micelles comprising cationic carrier units of the present disclosure) that are suitable for administration to a subject. As discussed above, micelles of the present disclosure can be homogeneous (i.e., all micelles comprise the same type of cationic carrier unit, with the same targeting moiety and the same payload). However, in other aspects, the micelles can comprise multiple targeting moieties, multiple payloads, etc.

The pharmaceutical compositions generally comprise a cationic carrier unit and/or micelle of the present disclosure and a pharmaceutically-acceptable excipient or carrier in a form suitable for administration to a subject. Pharmaceutically acceptable excipients or carriers are determined in part by the particular composition being administered, as well as by the particular method used to administer the composition.

There is a wide variety of suitable formulations of pharmaceutical compositions comprising micelles of the present disclosure (See, e.g., Remington's Pharmaceutical Sciences, Mack Publishing Co., Easton, Pa. 18th ed. (1990)). The pharmaceutical compositions are generally formulated sterile and in full compliance with all Good Manufacturing Practice (GMP) regulations of the U.S. Food and Drug Administration. In some aspects, the pharmaceutical composition comprises one or more micelles described herein.

In certain aspects, the micelles described herein are co-administered with one or more additional therapeutic agents, in a pharmaceutically acceptable carrier. In some aspects, the pharmaceutical composition comprising the micelles described herein is administered prior to administration of the additional therapeutic agent(s). In other aspects, the pharmaceutical composition comprising the micelles described herein is administered after the administration of the additional therapeutic agent(s). In further aspects, the pharmaceutical composition comprising the micelles described herein is administered concurrently with the additional therapeutic agent(s).

In some aspects, the pharmaceutical carrier is added following micelle formation. In other aspects, the pharmaceutical carrier is added before micelle formation.

Acceptable carriers, excipients, or stabilizers are nontoxic to recipients (e.g., animals or humans) at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as a polysorbate, such as polysorbate 20, polysorbate 60, polysorbate 65, or polysorbate 80 (e.g., TWEEN™), a poloxamer, including a block copolymer of PEO-PPO-PEO or PEO-PLGA-PEO, in which PEO is polyethylene oxide, PPO is polypropylene oxide, and PLGA is poly(lactic-co-glycolic) acid (e.g., PLURONIC™), or polyethylene glycol (PEG).

Examples of carriers or diluents include, but are not limited to, water, saline, Ringer's solutions, dextrose solution, and 5% human serum albumin. The use of such media and compounds for pharmaceutically active substances is well known in the art. Except insofar as any conventional media or compound is incompatible with the cationic carrier units or micelles disclosed herein, use thereof in the compositions is contemplated.

Supplementary therapeutic agents can also be incorporated into the compositions of the present disclosure. Typically, a pharmaceutical composition is formulated to be compatible with its intended route of administration. The micelles described herein can be administered by parenteral, topical, intravenous, oral, subcutaneous, intra-arterial, intradermal, transdermal, rectal, intracranial, intraperitoneal, intranasal, intratumoral, intramuscular route or as inhalants. In certain aspects, the pharmaceutical composition micelles described herein is administered intravenously, e.g. by injection. The micelles described herein can optionally be administered in combination with other therapeutic agents that are at least partly effective in treating the disease, disorder, or condition for which the micelles described herein are intended.

Solutions or suspensions can include the following components: a sterile diluent such as water, saline solution, fixed oils, polyethylene glycols, glycerine, propylene glycol or other synthetic solvents; antibacterial compounds such as benzyl alcohol or methyl parabens; antioxidants such as ascorbic acid or sodium bisulfite; chelating compounds such as ethylenediaminetetraacetic acid (EDTA); buffers such as acetates, citrates, or phosphates, and compounds for the adjustment of tonicity such as sodium chloride or dextrose. The pH can be adjusted with acids or bases, such as hydrochloric acid or sodium hydroxide. The preparation can be enclosed in ampoules, disposable syringes, or multiple dose vials made of glass or plastic.

Pharmaceutical compositions suitable for injectable use include sterile aqueous solutions (if water-soluble) or dispersions and sterile powders. For intravenous administration, suitable carriers include physiological saline, bacteriostatic water, Cremophor EL™ (BASF, Parsippany, N.J.) or phosphate buffered saline (PBS). The composition is generally sterile and fluid to the extent that easy syringeability exists. The carrier can be a solvent or dispersion medium containing, e.g., water, ethanol, polyol (e.g., glycerol, propylene glycol, and liquid polyethylene glycol, and the like), and suitable mixtures thereof. The proper fluidity can be maintained, e.g., by the use of a coating such as lecithin, by the maintenance of the required particle size in the case of dispersion and by the use of surfactants. Prevention of the action of microorganisms can be achieved by various antibacterial and antifungal compounds, e.g., parabens, chlorobutanol, phenol, ascorbic acid, thimerosal, and the like. If desired, isotonic compounds, e.g., sugars, polyalcohols such as mannitol, sorbitol, and sodium chloride can be added to the composition. Prolonged absorption of the injectable compositions can be brought about by including in the composition a compound which delays absorption, e.g., aluminum monostearate and gelatin.

Pharmaceutical compositions of the present disclosure can be sterilized by conventional, well known sterilization techniques. Aqueous solutions can be packaged for use or filtered under aseptic conditions and lyophilized, the lyophilized preparation being combined with a sterile aqueous solution prior to administration.

Sterile injectable solutions can be prepared by incorporating the micelles described herein in an effective amount and in an appropriate solvent with one or a combination of ingredients enumerated herein, as desired. Generally, dispersions are prepared by incorporating the micelles described herein into a sterile vehicle that contains a basic dispersion medium and any desired other ingredients. In the case of sterile powders for the preparation of sterile injectable solutions, methods of preparation are vacuum drying and freeze-drying that yields a powder of the active ingredient plus any additional desired ingredient from a previously sterile-filtered solution thereof. The micelles described herein can be administered in the form of a depot injection or implant preparation which can be formulated in such a manner to permit a sustained or pulsatile release of the micelles described herein.

Systemic administration of compositions comprising micelles described herein can also be by transmucosal means. For transmucosal administration, penetrants appropriate to the barrier to be permeated are used in the formulation. Such penetrants are generally known in the art, and include, e.g., for transmucosal administration, detergents, bile salts, and fusidic acid derivatives. Transmucosal administration can be accomplished through the use of, e.g., nasal sprays.

In certain aspects the pharmaceutical composition comprising micelles described herein is administered intravenously into a subject that would benefit from the pharmaceutical composition. In certain aspects, the composition is administered to the lymphatic system, e.g., by intralymphatic injection or by intranodal injection (see e.g., Senti et al., PNAS 105 (46): 17908 (2008)), or by intramuscular injection, by subcutaneous administration, by intratumoral injection, by direct injection into the thymus, or into the liver.

In certain aspects, the pharmaceutical composition comprising micelles described herein is administered as a liquid suspension. In certain aspects, the pharmaceutical composition is administered as a formulation that is capable of forming a depot following administration. In certain preferred aspects, the depot slowly releases the micelles described herein into circulation, or remains in depot form.

Typically, pharmaceutically-acceptable compositions are highly purified to be free of contaminants, are biocompatible and not toxic, and are suited to administration to a subject. If water is a constituent of the carrier, the water is highly purified and processed to be free of contaminants, e.g., endotoxins.

The pharmaceutically-acceptable carrier can be lactose, dextrose, sucrose, sorbitol, mannitol, starch, gum acacia, calcium phosphate, alginates, gelatin, calcium silicate, micro-crystalline cellulose, polyvinylpyrrolidone, cellulose, water, syrup, methyl cellulose, methylhydroxy benzoate, propylhydroxy benzoate, talc, magnesium stearate, and/or mineral oil, but is not limited thereto. The pharmaceutical composition can further include a lubricant, a wetting agent, a sweetener, a flavor enhancer, an emulsifying agent, a suspension agent, and/or a preservative.

The pharmaceutical compositions described herein comprise the micelles described herein and optionally a pharmaceutically active or therapeutic agent. The therapeutic agent can be a biological agent, a small molecule agent, or a nucleic acid agent.

Dosage forms are provided that comprise micelles described herein. In some aspects, the dosage form is formulated as a liquid suspension for intravenous injection.

The micelles disclosed herein or pharmaceutical composition comprising the micelles may be used concurrently with other drugs. To be specific, the micelles or pharmaceutical compositions of the present disclosure may be used together with medicaments such as hormonal therapeutic agents, chemotherapeutic agents, immunotherapeutic agents, medicaments inhibiting the action of cell growth factors or cell growth factor receptors and the like.

### VII. Methods of Treatment and Use

The present disclosure also provides methods of treating a disease or condition in a subject in need thereof comprising administering a micelle of the present disclosure or a combination thereof to the subject, e.g., a mammal, such as human subject. In some aspects, the present disclosure provides a method of treating a neurodegenerative disorder or cancer in a subject in need thereof, comprising administering to the subject a therapeutically effective amount of a micelle of the present disclosure, or a pharmaceutical composition of the present disclosure.

In some aspects, the micelles of the present disclosure can administered via intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion.

In some aspects, the micelles of the present disclosure can be used concurrently with other medicaments or treatment suitable for the treatment of the diseases and conditions disclosed herein.

The present disclosure also provides methods to encapsulate a payload for delivery, comprising incorporating the payload, e.g., an anionic payload such as a nucleic acid (e.g., an antimir) into a micelle of the present disclosure.

The present disclosure also provides methods to increase the resistance of a payload to degradation (e.g., nuclease-mediated degradation), comprising incorporating the payload, e.g., an anionic payload such as a nucleic acid (e.g., an antimir) into a micelle of the present disclosure.

In some aspects, the present disclosure provides methods of crossing blood brain barrier (BBB) comprising administering the micelles disclosed herein, e.g., micelles comprising tryptophan and/or tyrosine as a targeting moiety. A micelle of the present disclosure loaded with anti-miRNA can be targeted to a BBB receptor, e.g., LAT1, as disclosed above. Once the micelle is translocated across the BBB via receptor mediate transcytosis and undergoes cellular uptake by brain cells (e.g., neurons, astrocytes or microglia), the payload (e.g., an antimir) would be released and interact with an intracellular target (e.g., the antimir can bind to a target microRNA and trigger RNAse H mediated degradation).

In some aspects, encapsulation of the payload in a micelle of the present disclosure can increase the resistance of the payload to degradation at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 100% compared to the free payload (i.e., not in a micelle, e.g., free in solution).

In some aspects, encapsulation of the payload in a micelle of the present disclosure can increase the resistance of the payload to degradation at least about 2-fold, at least about 3-fold, at least about 4-fold, at least about 5-fold, at least about 6-fold, at least about 7-fold, at least about 8-fold, at least about 9-fold, at least about 10-fold, at least about 11-fold, at least about 12-fold, at least about 13-fold, at least about 14-fold, at least about 15-fold, at least about 16-fold, at least about 17-fold, at least about 18-fold, at least about 19-fold, at least about 20-fold, at least about 21-fold, at least about 22-fold, at least about 23-fold, at least about 24-fold, at least about 25-fold, at least about 26-fold, at least about 27-fold, at least about 28-fold, at least about 29-fold, or at least about 30-fold compared to the free payload.

The present disclosure also provides methods to increase the stability of a payload during administration (e.g., while in the subject's bloodstream) comprising incorporating the payload, e.g., an anionic payload such as a nucleic acid (e.g., an antimir) into a micelle of the present disclosure.

In some aspects, encapsulation of the payload in a micelle of the present disclosure can increase the stability (e.g., increase the resistance to nucleases) of the payload at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 100% compared to the free payload.

In some aspects, encapsulation of the payload in a micelle of the present disclosure can increase the stability (e.g., increase the resistance to nucleases) of the payload at least about 2-fold, at least about 3-fold, at least about 4-fold, at least about 5-fold, at least about 6-fold, at least about 7-fold, at least about 8-fold, at least about 9-fold, at least about 10-fold, at least about 11-fold, at least about 12-fold, at least about 13-fold, at least about 14-fold, at least about 15-fold, at least about 16-fold, at least about 17-fold, at least about 18-fold, at least about 19-fold, at least about 20-fold, at least about 21-fold, at least about 22-fold, at least about 23-fold, at least about 24-fold, at least about 25-fold, at least about 26-fold, at least about 27-fold, at least about 28-fold, at least about 29-fold, or at least about 30-fold compared to the free payload.

The present disclosure also provides methods to increase a payload's plasma half-life comprising incorporating the payload, e.g., an anionic payload such as a nucleic acid (e.g., an antimir) into a micelle of the present disclosure.

In some aspects, encapsulation of the payload in a micelle of the present disclosure can increase the plasma half-life of the payload at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 100%, at least about 200%, at least about 300%, at least about 400%, at least about 500%, at least about 600%, at least about 700%, at least about 800%, at least about 900%, at least about 1000%, at least about 1100%, at least about 1200%, at least about 1300%, at least about 1400%, at least about 1500%, at least about 1600%, at least about 1700%, at least about 1800%, at least about 1900%, or at least about 2000%, compared to the free payload.

In some aspects, encapsulation of the payload in a micelle of the present disclosure can increase the plasma half-life of the payload at least about 2-fold, at least about 3-fold, at least about 4-fold, at least about 5-fold, at least about 6-fold, at least about 7-fold, at least about 8-fold, at least about 9-fold, at least about 10-fold, at least about 11-fold, at least about 12-fold, at least about 13-fold, at least about 14-fold, at least about 15-fold, at least about 16-fold, at least about 17-fold, at least about 18-fold, at least about 19-fold, at least about 20-fold, at least about 21-fold, at least about 22-fold, at least about 23-fold, at least about 24-fold, at least about 25-fold, at least about 26-fold, at least about 27-fold, at least about 28-fold, at least about 29-fold, or at least about 30-fold compared to the free payload.

In some aspects, the encapsulated payload is an antimir disclosed herein, e.g., an antisense oligonucleotide of SEQ ID NO: 18, or a variant or derivative thereof (e.g., an oligonucleotide having at least about 70% identity to the antisense oligonucleotide of SEQ ID NO: 18) wherein the encapsulation of the antimir in a micelle of the present disclosure increases the plasma half-life of the antimir at least about 10-fold, at least about 12-fold, at least about 14-fold, at least about 16-fold, at least about 18-fold, or at least about 20-fold compared to the plasma half-life of the free antimir. In one particular aspect, the encapsulated payload is an antimir disclosed herein, e.g., an antisense oligonucleotide of SEQ ID NO: 18, or a variant or derivative thereof (e.g., an oligonucleotide having at least about 70% identity to the antisense oligonucleotide of SEQ ID NO: 18) wherein the encapsulation of the antimir in a micelle of the present disclosure increases the plasma half-life of the antimir at least about 20-fold compared to the plasma half-life of the free antimir. In some aspects, the plasma half-life of the antimir encapsulated in a micelle of the present disclosure is at least about 30 minutes, at least about 40 minutes, at least about 50 minutes, at least about 60 minutes, at least about 70 minutes, at least about 80 minutes, at least about 90 minutes, at least about 100 minutes, or at least about 120 minutes. In one particular aspects, the plasma half-life of the antimir (e.g., an antisense oligonucleotide of SEQ ID NO: 18) encapsulated in a micelle of the present disclosure is at least about 90 minutes.

The present disclosure also provides methods to increase the permeation, delivery, transit, or transport of a payload through a physiological barrier, e.g., the BBB or the plasma membrane, comprising incorporating the payload, e.g., an anionic payload such as a nucleic acid (e.g., an antimir) into a micelle of the present disclosure.

In some aspects, encapsulation of a payload in a micelle of the present disclosure can increase the permeation, delivery, transit, or transport of the payload through a physiological barrier, e.g., the BBB or the plasma membrane, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 100% compared to the free payload.

In some aspects, encapsulation of a payload in a micelle of the present disclosure can increase the permeation, delivery, transit, or transport of the payload through a physiological barrier, e.g., the BBB or the plasma membrane, at least about 2-fold, at least about 3-fold, at least about 4-fold, at least about 5-fold, at least about 6-fold, at least about 7-fold, at least about 8-fold, at least about 9-fold, at least about 10-fold, at least about 11-fold, at least about 12-fold, at least about 13-fold, at least about 14-fold, at least about 15-fold, at least about 16-fold, at least about 17-fold, at least about 18-fold, at least about 19-fold, at least about 20-fold, at least about 21-fold, at least about 22-fold, at least about 23-fold, at least about 24-fold, at least about 25-fold, at least about 26-fold, at least about 27-fold, at least about 28-fold, at least about 29-fold, or at least about 30-fold compared to the free payload.

In some aspects, the micelles of the present disclosure can be used to target stem cells, e.g., to deliver therapeutic molecules (e.g., therapeutic polynucleotides) or gene therapy components. In other aspects, the micelles of the present disclosure can be used to treat cancer. For example, micelles of the present disclosure can target a marker specific for a certain type of cancer, e.g., a glioma, breast cancer, pancreatic cancer, liver cancer, skin cancer, or cervical cancer, and carry as payload a therapeutic molecule (e.g., a therapeutic polynucleotide, a peptide, or a small molecule).

In specific aspects, the micelles of the present disclosure can be used to treat pancreatic cancer. In some aspects, the targeting moiety directing the micelles of the present disclosure to pancreatic tissues is a cyclic RGD peptide. In other aspects, the targeting moiety directing the micelles of the present disclosure to pancreatic tissues is a biomarker predominantly or exclusively expressed on the surface of normal or cancerous pancreatic cells. In some aspects, the payload of the micelle of the present disclosure is an oligonucleotide targeting K-Ras, wherein the delivery of the payload to pancreatic tissue effectively reduces the expression of K-Ras.

In some aspects, the micelles of the present disclosure can be used to treat or ameliorate the symptoms of a neurodegenerative disease, e.g., Alzheimer's disease. In some aspects, the micelles of the present disclosure comprise a payload, e.g., an antimir, targeting a molecule overexpressed in Alzheimer's disease neuronal tissue, e.g., miRNA-485-3p. Accordingly, in some aspects, the administration of a micelle of the present disclosure (e.g., a micelle comprising a LAT1 targeting moiety to effectively transport the micelle across the BBB and an antimir payload targeting miRNA-485-3p) to an Alzheimer's disease patient can prevent or ameliorate symptoms of Alzheimer's disease such as apoptosis, loss of mitochondrial function, or inflammation.

In some aspects, the present disclosure provides a method to reduce inflammation, e.g., neuroinflammation, in a subject suffering from a neurodegenerative disease (e.g., Alzheimer's disease) comprising administering to the subject a therapeutically effective amount of a micelle of the present disclosure, wherein the micelle comprises a therapeutic agent capable of effectively reducing inflammation, e.g., neuroinflammation, in the subject. In some aspects, the neuroinflammation is cortex inflammation. In some aspects, the neuroinflammation is hippocampus inflammation. In some aspects, the therapeutic agent is an antimir targeting miRNA-485-3p (e.g., an antimir of SEQ ID NO:18 or fragment or variant thereof) wherein the antimir can reduce the levels of miRNA-485-3p in the subject.

In some aspects, the administration of a micelle of the present disclosure to a subject suffering from a neurodegenerative disease (e.g., Alzheimer's disease) can decrease the level of neuroinflammation by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or about 100% of the neuroinflammation compared to the level of neuroinflammation observed in a subject or a population of subjects not treated with a micelle of the present disclosure.

In some aspects, the present disclosure provides a method to reduce amyloid plaque burden in a subject suffering from Alzheimer's disease comprising administering to the subject a therapeutically effective amount of a micelle of the present disclosure, wherein the micelle comprises a therapeutic agent capable of effectively reducing amyloid plaque burden in the subject. In some aspects, the therapeutic agent is an antimir targeting miRNA-485-3p (e.g., an antimir of SEQ ID NO:18 or fragment or variant thereof) wherein the antimir can reduce the levels of miRNA-485-3p in the subject.

In some aspects, the administration of a micelle of the present disclosure to a subject suffering from a neurodegenerative disease (e.g., Alzheimer's disease) can decrease at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or about 100% of the amyloid plaque burden in the subject compared to the amyloid plaque burden observed in a subject or a population of subjects not treated with a micelle of the present disclosure.

In some aspects, the present disclosure provides a method to recover and/or induce neurogenesis in a subject suffering from a neurodegenerative disease (e.g., Alzheimer's disease) comprising administering to the subject a therapeutically effective amount of a micelle of the present disclosure, wherein the micelle comprises a therapeutic agent capable of effectively recovering and/or inducing neurogenesis in the subject. In some aspects, the therapeutic agent is an antimir targeting miRNA-485-3p (e.g., an antimir of SEQ ID NO:18 or fragment or variant thereof) wherein the antimir can reduce the levels of miRNA-485-3p in the subject.

In some aspects, the administration of a micelle of the present disclosure to a subject suffering from a neurodegenerative disease (e.g., Alzheimer's disease) can recover and/or induce neurogenesis in the subject by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or about 100% compared to the level of neurogenesis observed in a subject or a population of subjects not treated with a micelle of the present disclosure.

In some aspects, the present disclosure provides a method to improve cognitive function in a subject suffering from a neurodegenerative disease (e.g., Alzheimer's disease) comprising administering to the subject a therapeutically effective amount of a micelle of the present disclosure, wherein the micelle comprises a therapeutic agent capable of effectively improving cognitive function in the subject. In some aspects, the therapeutic agent is an antimir targeting miRNA-485-3p (e.g., an antimir of SEQ ID NO:18 or fragment or variant thereof) wherein the antimir can reduce the levels of miRNA-485-3p in the subject.

In some aspects, the administration of a micelle of the present disclosure to a subject suffering from a neurodegenerative disease (e.g., Alzheimer's disease) can increase the cognitive function of the subject by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or about 100% compared to the cognitive function observed in a subject or a population of subjects not treated with a micelle of the present disclosure.

### VIII. Kits

The present disclosure also provides kits, or products of manufacture, comprising a cationic carrier unit, a micelle, or a pharmaceutical composition of the present disclosure and optionally instructions for use. In some aspects, the kit or product of manufacture comprises a cationic carrier unit, a micelle, or a pharmaceutical composition of the present disclosure in one or more containers. In some aspects, the kit or product of manufacture comprises a cationic carrier unit, a micelle, or a pharmaceutical composition of the present disclosure and a brochure. In some aspects, the kit or product of manufacture comprises a cationic carrier unit, a micelle, or a pharmaceutical composition of the present disclosure and instructions for use. One skilled in the art will readily recognize that a cationic carrier unit, a micelle, or a pharmaceutical composition of the present disclosure, or combinations thereof, can be readily incorporated into one of the established kit formats which are well known in the art.

In some aspects, the kit or product of manufacture comprises a cationic carrier unit of the present disclosure in dry form in a container (e.g., a glass vial), and optionally a vial with a solvent suitable to hydrate the dry the cationic carrier unit, and optionally instructions for the hydration of the cationic carrier unit and the formation of micelles. In some aspects, the kit or product of manufacture further comprises at least one additional container (e.g., a glass vial) with the micelle's anionic payload (e.g., an antisense oligonucleotide). In some aspects, the kit or product of manufacture comprises a cationic carrier unit of the present disclosure in a dry form and the micelle's anionic payload also in dry form in the same container, or in different containers. In some aspects, the kit or product of manufacture comprises a cationic carrier unit of the present disclosure in solution and the micelle's anionic payload also in solution in the same container, or in different containers. In some aspects, the kit or product of manufacture comprises a micelle of the present disclosure in solution, and instructions for use. In some aspects, the kit or product of manufacture comprises a micelle of the present disclosure in dry form, and instructions for use (e.g., instructions for reconstitution and administration).

The practice of the present disclosure will employ, unless otherwise indicated, conventional techniques of cell biology, cell culture, molecular biology, transgenic biology, microbiology, recombinant DNA, and immunology, which are within the skill of the art. Such techniques are explained fully in the literature. See, for example, Sambrook et al., ed. (1989) Molecular Cloning A Laboratory Manual (2nd ed.; Cold Spring Harbor Laboratory Press); Sambrook et al., ed. (1992) Molecular Cloning: A Laboratory Manual, (Cold Springs Harbor Laboratory, NY); D. N. Glover ed., (1985) DNA Cloning, Volumes I and II; Gait, ed. (1984) Oligonucleotide Synthesis; Mullis et al. U.S. Pat. No. 4,683,195; Hames and Higgins, eds. (1984) Nucleic Acid Hybridization; Hames and Higgins, eds. (1984) Transcription And Translation; Freshney (1987) Culture Of Animal Cells (Alan R. Liss, Inc.); Immobilized Cells And Enzymes (IRL Press) (1986); Perbal (1984) A Practical Guide To Molecular Cloning; the treatise, Methods In Enzymology (Academic Press, Inc., N.Y.); Miller and Calos eds. (1987) Gene Transfer Vectors For Mammalian Cells, (Cold Spring Harbor Laboratory); Wu et al., eds., Methods In Enzymology, Vols. 154 and 155; Mayer and Walker, eds. (1987) Immunochemical Methods In Cell And Molecular Biology (Academic Press, London); Weir and Blackwell, eds., (1986) Handbook Of Experimental Immunology, Volumes I-IV; Manipulating the Mouse Embryo, Cold Spring Harbor Laboratory Press, Cold Spring Harbor, N.Y., (1986); Crooke, Antisense drug Technology: Principles, Strategies and Applications, 2nd Ed. CRC Press (2007) and in Ausubel et al. (1989) Current Protocols in Molecular Biology (John Wiley and Sons, Baltimore, Md.).

All of the references cited above, as well as all references cited herein, are incorporated herein by reference in their entireties.

The following examples are offered by way of illustration and not by way of limitation.

### EXAMPLES

### Example 1

### (a) Synthesis of alkyne modified tyrosine

A mixture of N-(tert-butoxycarbonyl)-L-tyrosine methyl ester (Boc-Tyr-OMe) (0.5 g, 1.69 mmol) and K₂CO₃ (1.5 equiv., 2.54 mmol) in acetonitrile (4.0 ml) was added as dropwise to propargyl bromide (1.2 equiv., 2.03 mmol). The reaction mixture was heated at 60° C. for 16 hrs. After the reaction, the reaction mixture extracted by water with ethyl acetate (EA). Then, the organic layer was washed with brine solution. The crude material was purified by flash column (EA in hexane 10%). Next, the resulting product was dissolved in tetrahydrofuran (7.0 ml) and 6.0 M HCl (7.0 ml), and heated at 65° C. for 16 hrs. After, the dioxane was removed and extracted by EA. Then, aqueous NaOH (1.0 M) solution was added to the mixture until the pH value became 7. The reactant was concentrated by evaporator and centrifuged at 12,000 rpm at 0° C. The precipitate was washed with deionized water and lyophilized prior to use.

### (b) Synthesis of (methoxy or) azido-poly(ethylene glycol)-b-poly(L-lysine) (PEG-PLL)

Poly(ethylene glycol)-b-poly(L-lysine) was synthesized by ring opening polymerization of Lys(TFA)-NCA (N-trifluoroacetyl-L-lysine N-carboxyanhydride) with azido PEG (N₃-PEG) as a macroinitiator. In brief, N₃-PEG (600 mg, 0.12 mmol) and Lys(TFA)-NCA (1447 mg, 5.4 mmol) were separately dissolved in dimethylformamide (DMF) containing 1 M thiourea and DMF. A Lys(TFA)-NCA solution was dropped into the N₃-PEG solution by micro syringe needle, and the reaction mixture were stirred at 37° C. for 3 days. The reaction bottles were purged with argon (Ar) and vacuum, and all reactions were conducted under Ar atmosphere. After reaction, the mixture was precipitated into an excess amount of diethyl ether. Then, it was filtered and white powder was obtained after drying in vacuo. For the deprotection of the TFA group in PEG-PLL (TFA), N₃-PEG-PLL (500 mg) was dissolved in methanol (60 mL) and 1 N NaOH (6 mL) was dropped into the polymer solution with stirring. The mixture was maintained for 1 day with stirring at 37° C. The reaction mixture was dialyzed against 10 mM HEPES (N-2-hydroxyethylpiperazine-N′-2-ethanesulfonic acid) for 4 times and distilled water. White powder of N₃-PEG-PLL (NH₂) was obtained after lyophilization. PEG_{5K}-PLL (NH₃⁺/Cl⁻)₄₆, PEG_{5K}-PLL (NH₃⁺/Cl⁻)₅₈ and PEG_{5K}-PLL (NH₃⁺/Cl⁻)₈₀ were also polymerized though this synthetic method by controlling the feed amounts of reagents.

### Synthesis of (methoxy or) azido poly(ethylene glycol)-b-poly(L-lysine/nicotinamide/mercaptopropanamide) (PEG_{5K}-PLL₈₀ (NA₃₀/MPA₁₅)) (Compound A)

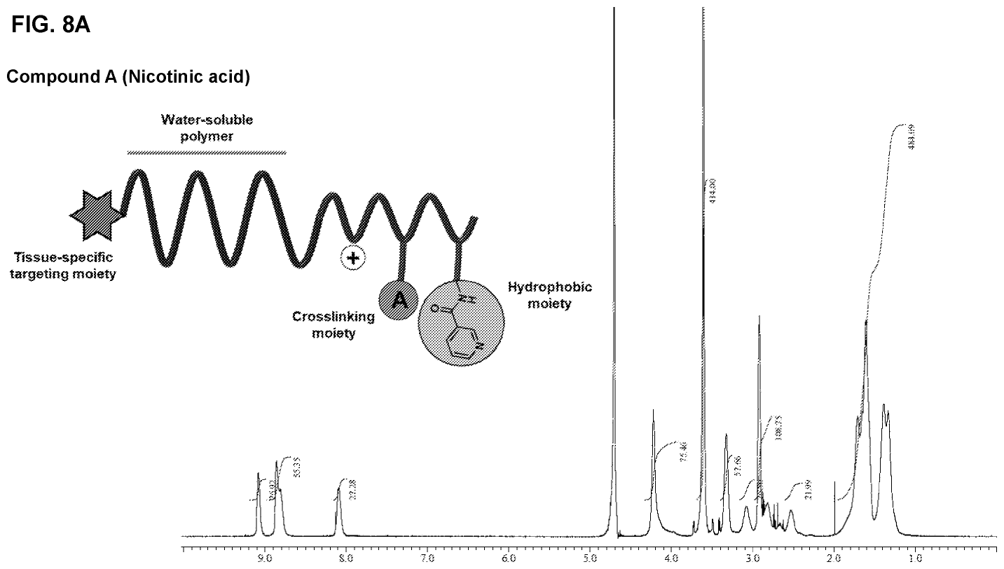

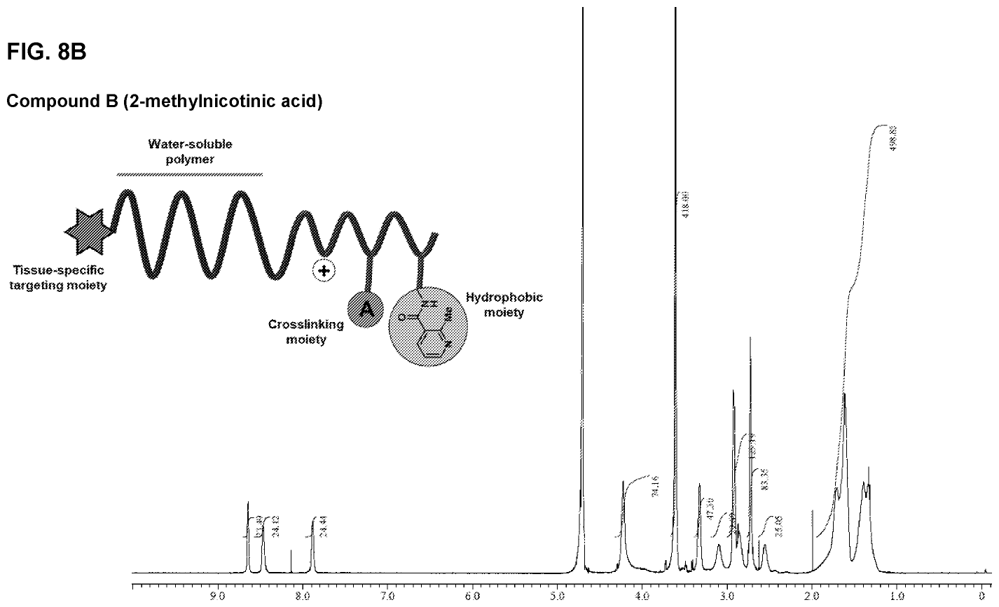

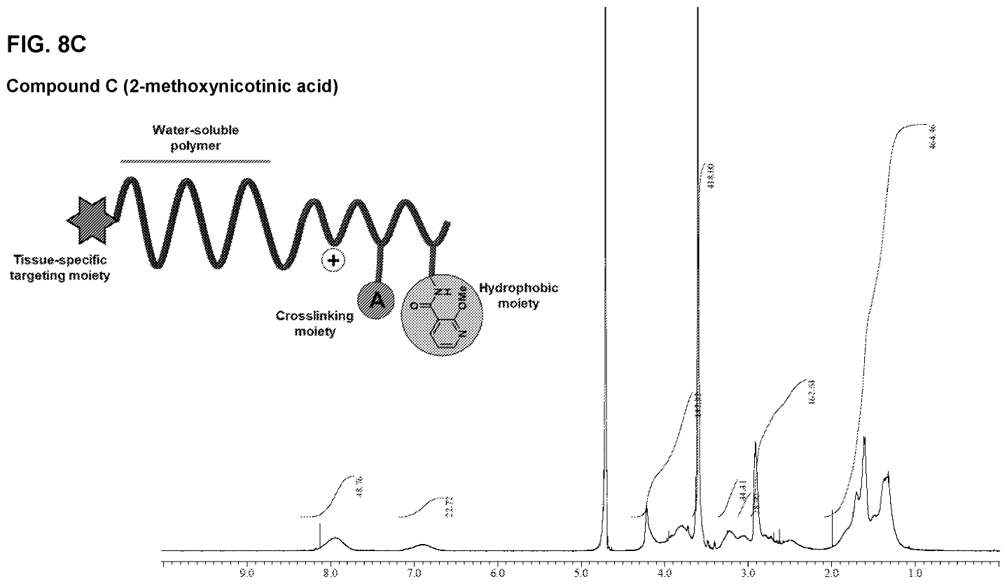

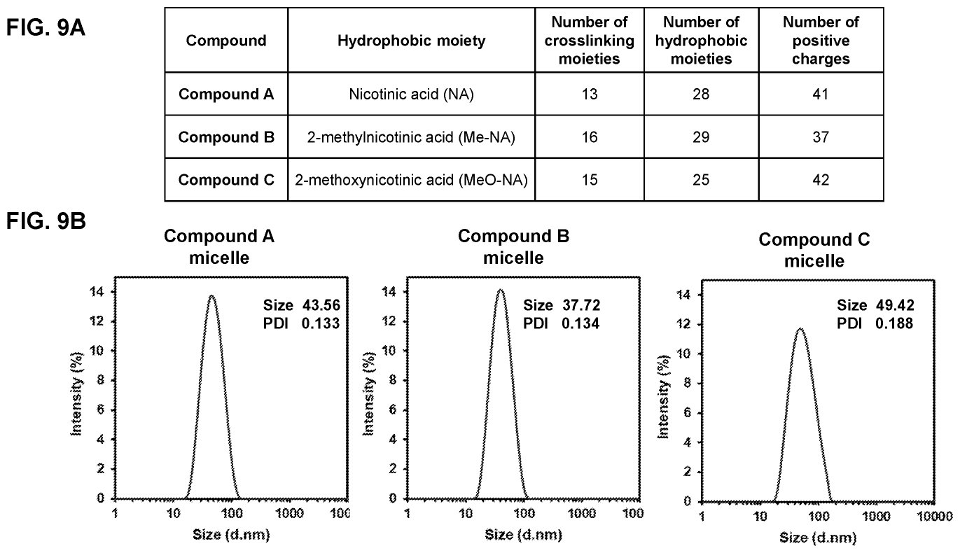

To enhance the stability of the micelle, nicotinamide and thiolated end groups were introduced into the polymer backbone. First, (methoxy or) azido-poly(ethylene glycol)-b-poly(L-lysine/nicotinamide/mercaptopropanamide) ((MeO— or) N₃-PEG_{5K}-PLL₈₀ (NA₃₀/MPA₁₅)) was synthesized by chemical modification of PEG_{5K}-PLL₈₀-NH₂ from nicotinic acid (NA) and 3,3′-dithiodipropionic acid (DTDPA) in the presence of EDC/NHS ((1-ethyl-3-(3-dimethylaminopropyl)carbodiimide/N-hydroxysuccinimide). PEG_{5K}-PLL₈₀-NH₂ (500.0 mg), NA (134.5 mg, 33 equiv.), DTDPA (66.2 mg, 10 equiv.), EDC (323.9 mg, 52 equiv.), NHS (194.5 mg, 52 equiv.), and triethylamine (TEA, 166.2 μL, 40 equiv.) were separately dissolved in 50 mL of dimethyl sulfoxide. The reaction mixture was maintained at 37° C. for 16 hours with stirring. After, the crude product was dialyzed (Molecular Weight Cut Off (MWCO)=7,000˜8,000 g/mol) against dionized water for 2 hours. 1,4-Dithiotritol (DTT, 145.8 mg, 31 equiv.) was directly added into the membrane to cleave the disulfide bond of the polymer side chain. The membrane was incubated for 30 min, dialyzed against deionized water for 2 hours, and dialyzed against deionized water for 24 hours. The solution was filtered with a syringe filter (0.45 μm) and lyophilized for 2 days. A schematic of PEG_{5K}-PLL₈₀ (NA₂₈/MPA₁₃) is shown as Compound A in FIG. 8A. The number of cross-linking moieties, hydrophobic moieties, and positive charges are set forth in FIG. 9A.

### Synthesis of (methoxy or) azido poly(ethylene glycol)-b-poly(L-lysine/2-methylnicotinamide/mercaptopropanamide) (PEG_{5K}-PLL₈₀ (Me-NA₃₀/MPA₁₅)) (Compound B)

To enhance the stability of the micelle, a nicotinamide derivative and thiolated end groups were introduced into the polymer backbone. First, (methoxy or) azido-poly(ethylene glycol)-b-poly(L-lysine/2-methylnicotinamide/mercaptopropanamide) ((MeO— or) N₃-PEG_{5K}-PLL₈₀ (Me-NA₃₀/MPA₁₅)) was synthesized by chemical modification of PEG_{5K}-PLL₈₀-NH₂ from 2-methylpyridine-3-carboxylic acid (Me—NA) and 3,3′-dithiodipropionic acid (DTDPA) in the presence of EDC/NHS. PEG_{5K}-PLL₈₀-NH₂ (300.0 mg), Me—NA (108.3 mg, 39 equiv.), DTDPA (43.7 mg, 10 equiv.), EDC (198.7 mg, 52 equiv.), NHS (119.3 mg, 52 equiv.), and triethylamine (TEA, 100 μL, 40 equiv.) were separately dissolved in 30 mL of dimethyl sulfoxide. The reaction mixture was maintained at 37° C. for 16 hours with stirring. After, the crude product was dialyzed (MWCO=7,000˜8,000 g/mol) against deionized water for 2 hours. 1,4-Dithiothreitol (DTT, 87.5 mg, 31 equiv.) was directly added into the membrane to cleave the disulfide bond of the polymer side chain. The membrane was incubated for 30 min, dialyzed against deionized water for 2 hours, and dialyzed against deionized water for 24 hours. The solution was filtered with syringe filter (0.45 μm) and lyophilized for 2 days. A schematic of PEG_{5K}-PLL₈₀ (Me—NA₂₉/MPA₁₆) is shown as Compound B (FIG. 8B). The number of cross-linking moieties, hydrophobic moieties, and positive charges are set forth in FIG. 9A.

### Synthesis of (methoxy or) azido poly(ethylene glycol)-b-poly(L-lysine/2-methoxynicotinamide/mercaptopropanamide) (PEG_{5K}-PLL₈₀ (MeO-NA₃₀/MPA₁₅)) (Compound C)

To enhance the stability of the micelle, a nicotinamide derivative and thiolated end groups were introduced into the polymer backbone. First, (methoxy or) azido-poly(ethylene glycol)-b-poly(L-lysine/2-methoxynicotinamide/mercaptopropanamide) ((MeO— or) N₃-PEG_{5K}-PLL₈₀ (MeO-NA₃₀/MPA₁₅)) was synthesized by chemical modification of PEG_{5K}-PLL₈₀-NH₂ from 2-methoxypyridine-3-carboxylic acid (MeO-NA) and 3,3′-dithiodipropionic acid (DTDPA) in the presence of EDC/NHS. PEG_{5K}-PLL₈₀-NH₂ (300.0 mg), MeO-NA (99.7 mg, 33 equiv.), DTDPA (43.7 mg, 10 equiv.), EDC (198.7 mg, 52 equiv.), NHS (119.3 mg, 52 equiv.), and triethylamine (TEA, 100 μL, 40 equiv.) were separately dissolved in 30 mL of dimethyl sulfoxide. The reaction mixture was maintained at 37° C. for 16 hours with stirring. After, the crude product was dialyzed (MWCO=7,000˜8,000 g/mol) against deionized water for 2 hours. 1,4-Dithiothreitol (DTT, 87.5 mg, 31 equiv.) was directly added into the membrane to cleave the disulfide bond of the polymer side chain. The membrane was incubated for 30 min, dialyzed against deionized water for 2 hours, and dialyzed against deionized water for 24 hours. The solution was filtered with a syringe filter (0.45 μm) and lyophilized for 2 days. A schematic of PEG_{5K}-PLL₈₀ (MeQ-NA₂₅/MPA₁₅) is shown as Compound C in FIG. 8C. The number of cross-linking moieties, hydrophobic moieties, and positive charges are set forth in FIG. 9A.

### Synthesis of (methoxy or) azido poly(ethylene glycol)-b-poly(L-lysine/nicotinamide/mercaptopropanamide) (PEG_{5K}-PLL₈₀ (NA₆/MPA₃₀)) (Compound D)

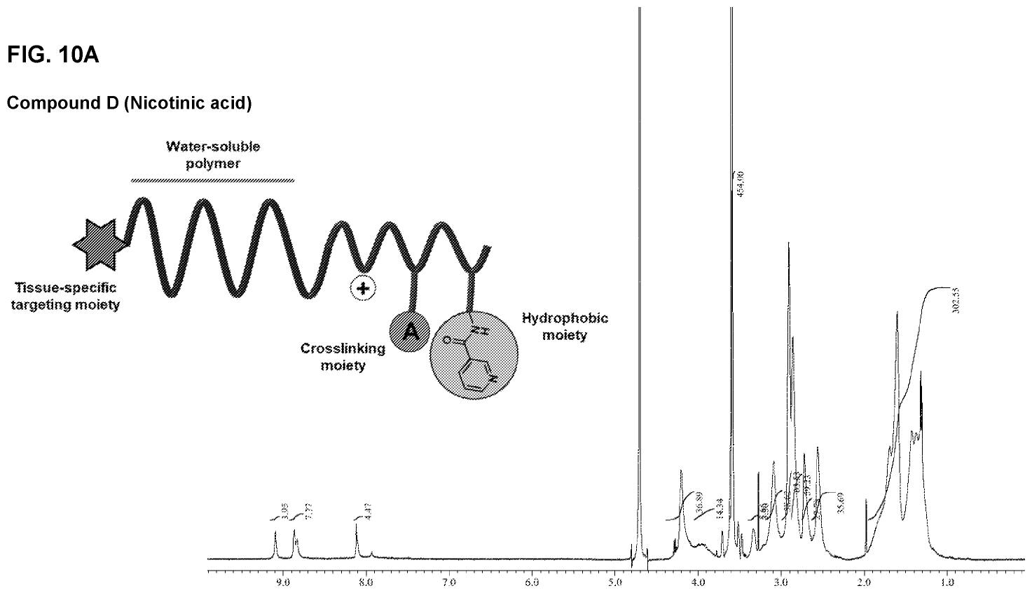

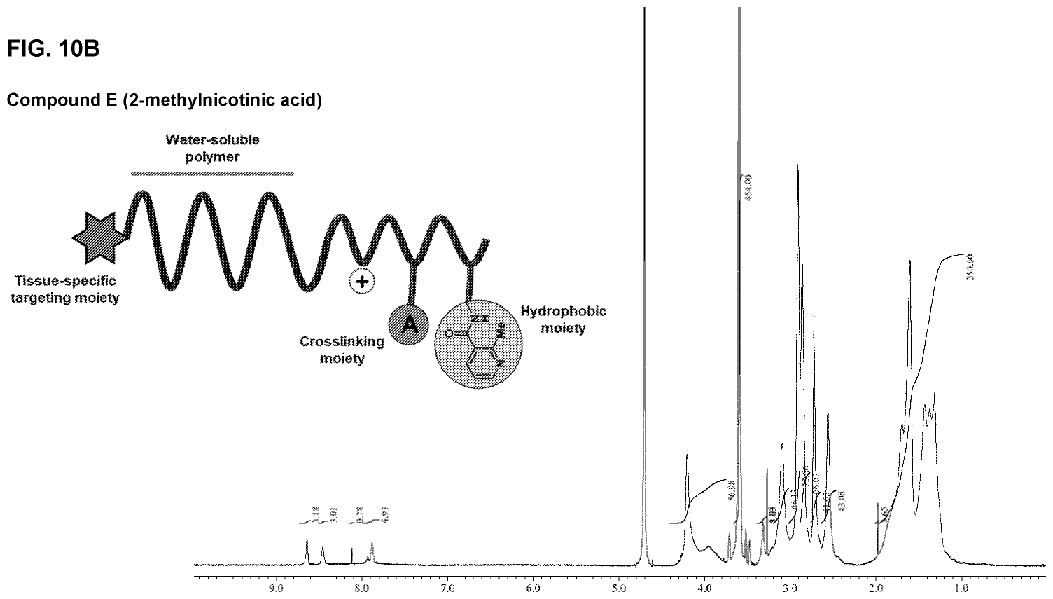

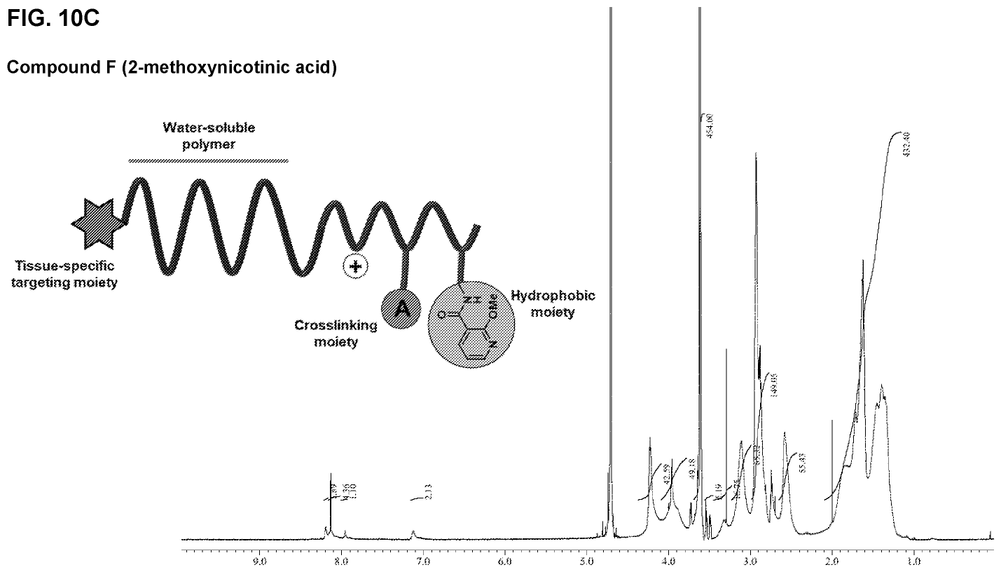

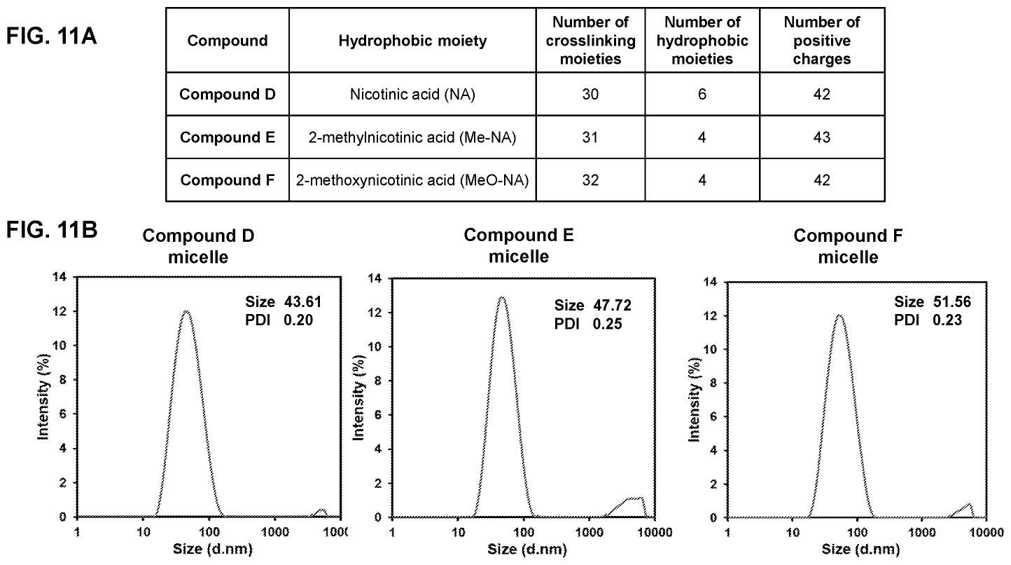

To enhance the stability of the micelle, nicotinamide and thiolated end groups were introduced into the polymer backbone. First, (methoxy or) azido-poly(ethylene glycol)-b-poly(L-lysine/nicotinamide/mercaptopropanamide) ((MeO— or) N₃-PEG_{5K}-PLL₈₀ (NA₅/MPA₃₀)) was synthesized by chemical modification of PEG_{5K}-PLL₈₀-NH₂ from nicotinic acid (NA) and 3,3′-dithiodipropionic acid (DTDPA) in the presence of EDC/NHS. PEG_{5K}-PLL₈₀-NH₂ (500.0 mg), NA (20.2 mg, 5 equiv.), DTDPA (184.3 mg, 26.7 equiv.), EDC (260.3 mg, 41.3 equiv.), NHS (156.1 mg, 41.3 equiv.), and triethylamine (TEA, 178 μL, 36 equiv.) were separately dissolved in 50 mL of dimethyl sulfoxide. The reaction mixture was maintained at 37° C. for 16 hours with stirring. After, the crude product was dialyzed (MWCO=7,000˜8,000 g/mol) against deionized water for 2 hours. 1,4-Dithiotritol (DTT, 145.8 mg, 31 equiv.) was directly added into the membrane to cleave the disulfide bond of the polymer side chain. The membrane was incubated for 30 min, dialyzed against deionized water for 2 hours, and dialyzed against deionized water for 24 hours. The solution was filtered with a syringe filter (0.45 μm) and lyophilized for 2 days. A schematic of PEG_{5K}-PLL₈₀ (NA₂₈/MPA₁₃) is shown as Compound D in FIG. 10A. The number of cross-linking moieties, hydrophobic moieties, and positive charges are set forth in FIG. 11A.

### Synthesis of (methoxy or) azido poly(ethylene glycol)-b-poly(L-lysine/2-methylnicotinamide/mercaptopropanamide) (PEG_{5K}-PLL₈₀ (Me—NA₄/MPA₃₁)) (Compound E)

To enhance the stability of the micelle, a nicotinamide derivative and thiolated end groups were introduced into the polymer backbone. First, (methoxy or) azido-poly(ethylene glycol)-b-poly(L-lysine/2-methylnicotinamide/mercaptopropanamide) ((MeO— or) N₃-PEG_{5K}-PLL₈₀ (Me—NAs/MPA₃₀)) was synthesized by chemical modification of PEG_{5K}-PLL₈₀-NH₂ from 2-methylpyridine-3-carboxylic acid (Me—NA) and 3,3′-dithiodipropionic acid (DTDPA) in the presence of EDC/NHS. PEG_{5K}-PLL₈₀-NH₂ (500.0 mg), NA (22.5 mg, 5 equiv.), DTDPA (184.3 mg, 26.7 equiv.), EDC (260.3 mg, 41.3 equiv.), NHS (156.1 mg, 41.3 equiv.), and triethylamine (TEA, 178 μL, 36 equiv.) were separately dissolved in 30 mL of dimethyl sulfoxide. The reaction mixture was maintained at 37° C. for 16 hours with stirring. After, the crude product was dialyzed (MWCO=7,000˜8,000 g/mol) against deionized water for 2 hours. 1,4-Dithiotritol (DTT, 145.8 mg, 31 equiv.) was directly added into the membrane to cleave the disulfide bond of the polymer side chain. The membrane was incubated for 30 min, dialyzed against deionized water for 2 hours, and dialyzed against deionized water for 24 hours. The solution was filtered with a syringe filter (0.45 μm) and lyophilized for 2 days. A schematic of PEG_{5K}-PLL₈₀ (Me—NA₂₉/MPA₁₆) is shown as Compound E in FIG. 10B. The number of cross-linking moieties, hydrophobic moieties, and positive charges are set forth in FIG. 11A.

### Synthesis of (methoxy or) azido poly(ethylene glycol)-b-poly(L-lysine/2-methoxynicotinamide/mercaptopropanamide) (PEG_{5K}-PLL₈₀ (MeO-NA₄/MPA₃₂)) (Compound F)

To enhance the stability of the micelle, a nicotinamide derivative and thiolated end groups were introduced into the polymer backbone. First, (methoxy or) azido-poly(ethylene glycol)-b-poly(L-lysine/2-methoxynicotinamide/mercaptopropanamide) ((MeO— or) N₃-PEG_{5K}-PLL₈₀ (MeO-NA₅/MPA₃₀)) was synthesized by chemical modification of PEG_{5K}-PLL₈₀-NH₂ from 2-methoxypyridine-3-carboxylic acid (MeO-NA) and 3,3′-dithiodipropionic acid (DTDPA) in the presence of EDC/NHS. PEG_{5K}-PLL₈₀-NH₂ (500.0 mg), MeO-NA (99.7 mg, 33 equiv.), DTDPA (184.3 mg, 26.7 equiv.), EDC (260.3 mg, 41.3 equiv.), NHS (156.1 mg, 41.3 equiv.), and triethylamine (TEA, 178 μL, 36 equiv.) were separately dissolved in 30 mL of dimethyl sulfoxide. The reaction mixture was maintained at 37° C. for 16 hours with stirring. After, the crude product was dialyzed (MWCO=7,000˜8,000 g/mol) against deionized water for 2 hours. 1,4-Dithiothreitol (DTT, 145.8 mg, 31 equiv.) was directly added into the membrane to cleave the disulfide bond of the polymer side chain. The membrane was incubated for 30 min, dialyzed against deionized water for 2 hours, and dialyzed against deionized water for 24 hours. The solution was filtered with a syringe filter (0.45 μm) and lyophilized for 2 days. A schematic of PEG_{5K}-PLL₈₀ (MeO-NA₂₅/MPA₁₅) is shown as Compound F in FIG. 10C. The number of cross-linking moieties, hydrophobic moieties, and positive charges are set forth in FIG. 11A.

### (c) Synthesis of phenylalanine-poly(ethylene glycol)-b-poly(L-lysine/nicotinamide/mercaptopropanamide) (Phe-PEG-PLL (NA/MPA))

In order to target brain endothelial tissue in the blood stream, as a LAT1 targeting amino acid, phenyl alanine was introduced by click reaction between N₃-PEG-PLL (NA/MPA) and alkyne-modified tyrosine in the presence of a copper catalyst. In brief, N₃-PEG-PLL (Nic/SH) (30 mg, 3.2 μmol) and alkyne-modified phenylalanine (1.31 mg, 6.4 μmol) were dissolved in deionized water. Then, CuSO₄·H₂O (0.172 mg, 0.69 μmol) and ascorbic acid (0.3 mg, 1.7 μmol) were added into the mixture solution. The reaction mixture was maintained with stirring for 16 hours at room temperature. After the reaction, the mixture was transfered into a dialysis membrane (MWCO=7,000 g/mol) and dialyzed against deionized water for 1 day. The final product was obtained after lyophilization.

### Example 2

Once the cationic carrier units of Compounds A, B, and C were generated as described in Example 1, micelles were produced. The micelles described in the present example comprised cationic carrier units combined with an antisense oligonucleotide (ASO) as a payload.

### (a) Micelle formulated with ASO and PEG_{5K}-PLL₈₀ (NA₂₈/MPA₁₃) (Compound A)

Nano sized polyion complex (PIC) micelles were prepared by mixing of MeO— or Phe-PEG_{5K}-PLL₈₀ (NA₂₈/MPA₁₃) and ASO. PEG_{5K}-PLL₈₀ (NA₂₈/MPA₁₃) was dissolved in 200 mM of DTT solution (in 10 mM of HEPES buffer) at 0.78 mg/mL concentration. Then, the ASO solution (22.5 μM) in RNase free water was mixed with the polymer solution at 2:1 (v/v) ratio of ASO to polymer. The mixing ratios of the polymer to the ASO were determined by the optimization of micelle forming conditions between amine in the polymer to phosphate in the ASO (N to P ratio was 1.6). The mixture of the polymer and the ASO was vigorously mixed for 1 min by multi-vortex at 3000 rpm, and kept at room temperature for 30 min to stabilize the micelles. Particle size distribution was measured by Zeta-sizer with 634 nm wavelength (FIG. 9B). Final micelles (15 μM of ASO Conc.) were stored at 4° C. prior to use.

### (b) Micelle formulated with ASO and PEG_{5K}-PLL₈₀ (Me—NA₂₉/MPA₁₆) (Compound B)

Nano sized PIC micelles were prepared by mixing of MeO— or Phe-PEG_{5K}-PLL₈₀ (Me—NA₂₉/MPA₁₆) and ASO. PEG_{5K}-PLL₈₀ (Me—NA₂₉/MPA₁₆) was dissolved in 200 mM of DTT solution (in 10 mM of HEPES buffer) at 0.86 mg/mL concentration. Then, the ASO solution (22.5 μM) in RNase free water was mixed with the polymer solution at 2:1 (v/v) ratio of ASO to polymer. The mixing ratios of the polymer to the ASO were determined by the optimization of micelle forming condition between amine in the polymer to phosphate in the ASO (N to P ratio was 1.7). The mixture of the polymer and the ASO was vigorously mixed for 1 min by multi-vortex at 3000 rpm, and kept at room temperature for 30 min to stabilize the micelles. Particle size distribution was measured by Zeta-sizer with 634 nm wavelength (FIG. 9B). Final micelles (15 μM of ASO Conc.) were stored at 4° C. prior to use.

### (c) Micelle formulated with ASO and PEG_{5K}-PLL₈₀ (MeO-NA₂₅/MPA₁₅) (Compound C)

Nano sized PIC micelles were prepared by mixing of MeO— or Phe-PEG_{5K}-PLL₈₀ (MeO-NA₂₅/MPA₁₅) and ASO. PEG_{5K}-PLL₈₀ (MeO-NA₂₅/MPA₁₅) was dissolved in 200 mM of DTT solution (in 10 mM of HEPES buffer) at 0.78 mg/mL concentration. Then, the ASO solution (22.5 M) in RNase free water was mixed with the polymer solution at 2:1 (v/v) ratio of ASO to polymer. The mixing ratios of the polymer to the ASO were determined by the optimization of micelle forming condition between amine in the polymer to phosphate in the ASO (N to P ratio was 1.6). The mixture of the polymer and the ASO was vigorously mixed for 1 min by multi-vortex at 3000 rpm and kept at room temperature for 30 min to stabilize the micelles. Particle size distribution was measured by Zeta-sizer with 634 nm wavelength (FIG. 9B). Final micelles (15 μM of ASO Conc.) were stored at 4° C. prior to use.

Once the cationic carrier units of Compounds D, E, and F were generated as described in Example 1, micelles were produced. The micelles described in the present example comprised cationic carrier units combined with an mRNA as payload. The luciferase gene encoding mRNA, which had a length of 1700 nucleotides (nt), was used for mRNA formulation.

### (a) Micelle formulated with mRNA and PEG_{5K}-PLL₈₀ (NA₆/MPA₃₀) (Compound D)

Nano sized PIC micelles were prepared by mixing MeO— or Phe-PEG_{5K}-PLL₈₀ (NA₆/MPA₃₀) and mRNA. PEG_{5K}-PLL₈₀ (NA₆/MPA₃₀) was dissolved in 200 mM of DTT solution (in 10 mM of HEPES buffer) at 2.5 mg/mL concentration. Next, an mRNA solution (0.38 μM) in RNase free water was mixed with the polymer solution at 2:1 (v/v) ratio of mRNA to polymer. The mixing ratios of polymer to mRNA was determined by the optimization of micelle forming conditions between amine in polymer to phosphate in mRNA (N to P ratio was 4). The mixture of polymer and mRNA was vigorously mixed for 1 min by multi-vortex at 3000 rpm, and kept at room temperature for 30 min to stabilize the micelles. Particle size distribution was measured by Zeta-sizer with 634 nm wavelength (FIG. 11B). Final micelles (0.25 μM of mRNA Conc.) were stored at 4° C. prior to use.

### (b) Micelle formulated with mRNA and PEG_{5K}-PLL₈₀ (Me—NA₄/MPA₃₁) (Compound E)

Nano sized PIC micelles were prepared by mixing of MeO— or Phe-PEG_{5K}-PLL₈₀ (Me—NA₄/MPA₃₁) and mRNA. PEG_{5K}-PLL₈₀ (Me—NA₄/MPA₃₁) was dissolved in 200 mM of DTT solution (in 10 mM of HEPES buffer) at 2.58 mg/mL concentration. Next, an mRNA solution (0.38 μM) in RNase free water was mixed with the polymer solution at 2:1 (v/v) ratio of mRNA to polymer. The mixing ratios of polymer to mRNA was determined by the optimization of micelle forming conditions between amine in polymer to phosphate in mRNA (N to P ratio was 4). The mixture of polymer and mRNA was vigorously mixed for 1 min by multi-vortex at 3000 rpm, and kept at room temperature for 30 min to stabilize the micelles. Particle size distribution was measured by Zeta-sizer with 634 nm wavelength (FIG. 11B). Final micelles (0.25 μM of mRNA Conc.) were stored at 4° C. prior to use.

### (c) Micelle formulated with mRNA and PEG_{5K}-PLL₈₀ (MeO-NA₄/MPA₃₂) (Compound F)

Nano sized PIC micelles were prepared by mixing of MeO— or Phe-PEG_{5K}-PLL₈₀ (MeO-NA₄/MPA₃₂) and mRNA. PEG_{5K}-PLL₈₀ (MeO-NA₄/MPA₃₂) was dissolved in 200 mM of DTT solution (in 10 mM of HEPES buffer) at 2.48 mg/mL concentration. Next, an mRNA solution (0.38 μM) in RNase free water was mixed with the polymer solution at 2:1 (v/v) ratio of mRNA to polymer. The mixing ratios of polymer to mRNA was determined by the optimization of micelle forming conditions between amine in polymer to phosphate in mRNA (N to P ratio was 4). The mixture of polymer and mRNA was vigorously mixed for 1 min by multi-vortex at 3000 rpm, and kept at room temperature for 30 min to stabilize micelles. Particle size distribution was measured by Zeta-sizer with 634 nm wavelength (FIG. 11B). Final micelles (0.25 μM of mRNA Conc.) were stored at 4° C. prior to use.

### Example 3

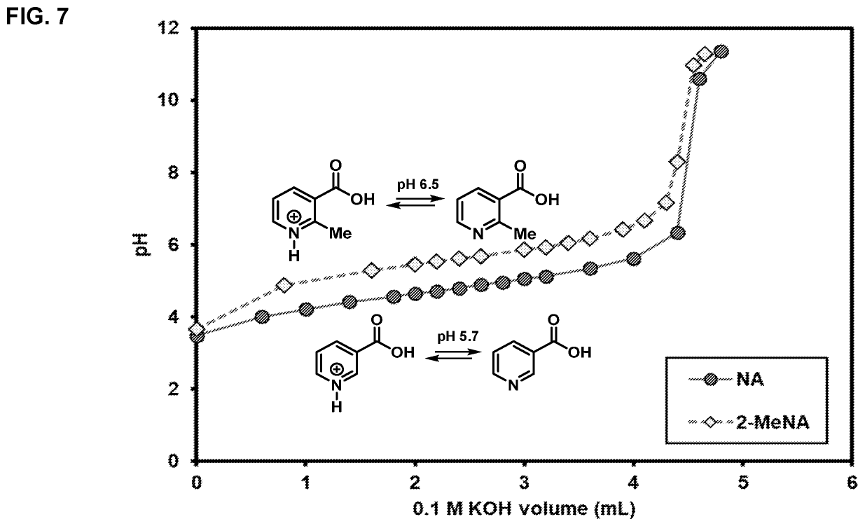

The pKa of nicotinic acid and 2-methylnicotinic acid were calculated by trituration with a 0.1 M KOH solution. 2-Methoxynicotinic acid (MeO-NA) did not dissolve in water, so the MeO-NA pKa could not be determined. The nicotinic acid (NA) and 2-methylnicotinic acid (Me-NA) samples were prepared as a 0.1 M solution, and then each sample was measured for initial pH. The prepared NA and Me—NA solutions were titrated by 0.1 M KOH. The obtained pKa's of the NA and Me—NA solutions following titration were calculated to be 5.7 and 6.5, respectively (FIG. 7).

### Example 4

The SH-SY5Y cells were incubated in Minimal Essential Medium/Earle's Balanced Salts (MEM/EBSS, Cytiva, USA) supplemented with 10% FBS (fetal bovine serum) and antibiotics (100 μg/mL of streptomycin and 100 U/mL of penicillin, Gibco, USA) in a humidified 5% CO₂ incubator at 37° C.

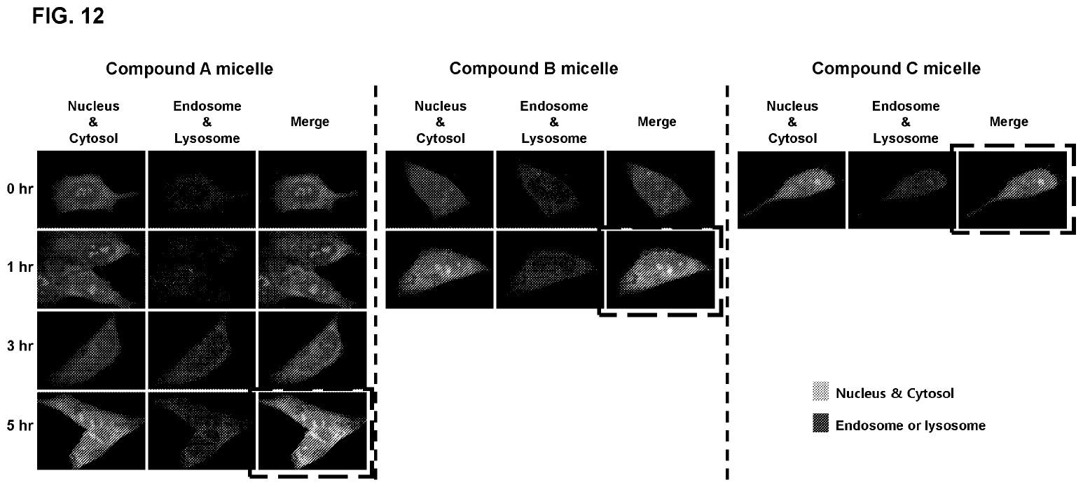

The cells (1×10³) were seeded on a confocal dish (35ϕ) and further incubated in a CO₂ incubator. After 24 hours, each sample (compound A micelles, compound B micelles, compound C micelles) was separately added to the seeded cells at 300 nM of ASO concentration. After 30 min of incubation, the cells were washed with PBS to adjust the uptake amounts of micelles and further incubated for 0, 1, 3, and 5 hours. For observation of endosome or lysosome formation, acridine orange staining solution (5 μM) was added to the cells and fixed with 4% paraformaldehyde. The morphological change of the cells was observed by confocal microscopy (Green: Ex/Em=502-525 nm, Red: Ex/Em=610-640 nm). The results are shown in FIG. 12.

It is to be appreciated that the Detailed Description section, and not the Summary and Abstract sections, is intended to be used to interpret the claims. The Summary and Abstract sections may set forth one or more but not all exemplary aspects of the present disclosure as contemplated by the inventor(s), and thus, are not intended to limit the present disclosure and the appended claims in any way.

The present disclosure has been described above with the aid of functional building blocks illustrating the implementation of specified functions and relationships thereof. The boundaries of these functional building blocks have been arbitrarily defined herein for the convenience of the description. Alternate boundaries can be defined so long as the specified functions and relationships thereof are appropriately performed.

The foregoing description of the specific embodiments will so fully reveal the general nature of the disclosure that others can, by applying knowledge within the skill of the art, readily modify and/or adapt for various applications such specific embodiments, without undue experimentation, without departing from the general concept of the present disclosure. Therefore, such adaptations and modifications are intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

The breadth and scope of the present disclosure should not be limited by any of the above-described exemplary embodiments, but should be defined only in accordance with the following claims and their equivalents.

What is claimed is:

1. A cationic carrier unit comprising 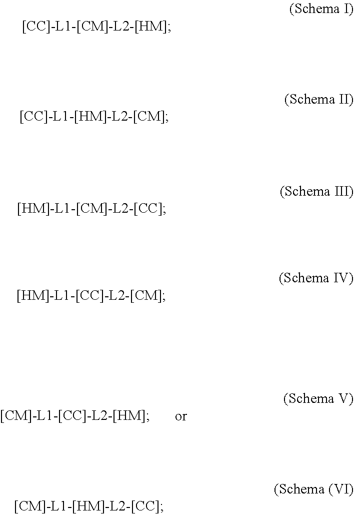
wherein
CC is a positively charged carrier moiety;
CM is a crosslinking moiety;
HM is a nicotinic acid derivative; and,
L1 and L2 are independently optional linkers, and
wherein the number of HM is less than about 50% relative to [CC] and [CM].

2. The cationic carrier unit of claim 1, wherein the nicotinic acid derivative comprises an electron donating group or an electron withdrawing group.

3. The cationic carrier unit of claim 1 or 2, wherein the nicotinic acid derivative comprises 2-methylnicotinic acid (Me—NA), 2-methoxynicotinic acid (MeO-NA), or any combination thereof.

4. The cationic carrier unit of claim 1 or 2, wherein the nicotinic acid derivative comprises 2-methylnicotinic acid (Me—NA).

5. The cationic carrier unit of claim 1 or 2, wherein the nicotinic acid derivative comprises 2-methoxynicotinic acid (MeO-NA).

6. The cationic carrier unit of any one of claims 1 to 5, wherein the number of HM is less than about 45%, about 40%, about 35%, about 30%, about 25%, about 20%, about 15%, about 10%, about 9%, about 8%, about 7%, about 6%, about 5%, about 4%, about 3%, about 2%, or about 1% relative to [CC] and [CM].

7. The cationic carrier unit of any one of claims 1 to 5, wherein the number of HM is between about 50% and about 1%, about 50% and about 5%, about 50% and about 10%, about 50% and about 15%, about 50% and about 20%, about 50% and about 25%, about 50% and about 30%, about 50% and about 35%, about 50% and about 40%, about 50% and about 45%, about 45% and about 1%, about 45% and about 5%, about 45% and about 10%, about 45% and about 15%, about 45% and about 20%, about 45% and about 25%, about 45% and about 30%, about 45% and about 35%, about 45% and about 40%, about 40% and 1%, about 50% and about 5%, about 40% and about 10%, about 40% and about 15%, about 40% and about 20%, about 40% and about 25%, about 40% and about 30%, about 40% and about 35%, about 35% and about 1%, about 35% and about 5%, about 35% and about 10%, about 35% and about 15%, about 35% and about 20%, about 35% and about 25%, about 35% and about 30%, about 30% and about 1%, about 30% and about 5%, about 30% and about 10%, about 30% and about 15%, about 30 and about 20%, about 30% and about 25%, about 25% and about 1%, about 25% and about 5%, about 25% and about 10%, about 25% and about 15%, about 25% and about 20%, about 20% and about 1%, about 20% and about 5%, about 20% and about 10%, about 20% and about 15%, about 15% and about 1%, about 15% and about 5%, about 15% and about 10%, about 10% to about 1%, about 10% to about 5%, or about 5% to about 1% relative to [CC] and [CM].

8. The cationic carrier unit of any one of claims 1 to 5, wherein the number of HM is between about 50% and about 40%, about 40% and about 30%, about 30% and about 20%, about 20% and about 10%, about 10% and about 5%, and about 5% and about 1%.

9. The cationic carrier unit of any one of claims 1 to 5, wherein the number of HM is about 50%, about 45%, about 40%, about 35%, about 30%, about 25%, about 20%, about 15%, about 10%, about 5%, or about 1%.

10. The cationic carrier unit of any one of claims 1 to 9, wherein the cationic carrier unit is capable of interacting with an anionic payload.

11. The cationic carrier unit of claim 10, wherein the anionic payload comprises a nucleotide sequence having less than 200 nucleotides in length.

12. The cationic carrier unit of claim 10, wherein the anionic payload comprises a nucleotide sequence having less than about 150, about 140, about 130, about 120, about 110, about 100, about 90, about 80, about 70, about 60, about 50, about 40, about 30, about 25, about 24, about 23, about 22, about 21, about 20, about 19, about 18, about 17, about 16, about 15, about 14, about 13, about 12, about 11, or about 10 nucleotides in length.

13. The cationic carrier unit of claim 10, wherein the anionic payload comprises a nucleotide sequence having from about 30 to about 10, from about 25 to about 11, from about 30 to about 15, from about 25 to about 15, from about 24 to about 15, or from about 23 to about 15 nucleotides in length.

14. The cationic carrier unit of claim 10, wherein the anionic payload comprises a nucleotide sequence having about 30, about 29, about 28, about 27, about 26, about 25, about 24, about 23, about 22, about 21, about 20, about 19, about 18, about 17, about 16, about 15, about 14, or about 13 nucleotides in length.

15. The cationic carrier unit of claim 10, wherein the anionic payload comprises a nucleotide sequence having about 22 nucleotides in length.

16. The cationic carrier unit of claims 10 to 15, wherein the anionic paylod comprises mRNA, miRNA, miRNA sponge, tough decoy miRNA, antimir, small RNA, IRNA, siRNA, shRNA, gDNA, cDNA, pDNA, PNA, BNA, antisense oligonucleotide (ASO), aptamer, cyclic dinucleotide, or any combination thereof.

17. The cationic carrier unit of any one of claims 1 to 16, which further comprises a water-soluble polymer (WP).

18. The cationic carrier unit of claim 17, wherein the water-soluble polymer is attached to [CC], [HM], or [CM].

19. The cationic carrier unit of claim 17 or 18, wherein the water-soluble polymer is attached to the N terminus of [CC], [HM], or [CM].

20. The cationic carrier unit of claim 17 or 18, wherein the water-soluble polymer is attached to the C terminus of [CC], [HM], or [CM].

21. The cationic carrier unit of any one of claims 17 to 19, which comprises:

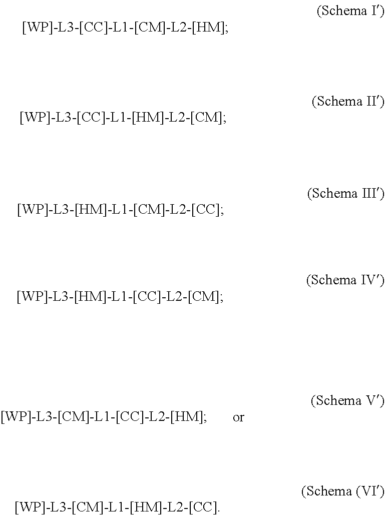

22. The cationic carrier unit of any one of claims 17 to 21, wherein the water-soluble polymer comprises poly(alkylene glycols), poly(oxyethylated polyol), poly(olefinic alcohol), poly(vinylpyrrolidone), poly(hydroxyalkylmethacrylamide), poly(hydroxyalkylmethacrylate), poly(saccharides), poly(α-hydroxy acid), poly(vinyl alcohol), polyglycerol, polyphosphazene, polyoxazolines (POZ) poly(N-acryloylmorpholine), or any combinations thereof.

23. The cationic carrier unit of any one of claims 17 to 21, wherein the water-soluble polymer comprises polyethylene glycol (PEG), polyglycerol, or poly(propylene glycol) (PPG).

24. The cationic carrier unit of any one of claims 17 to 23, wherein the water-soluble polymer comprises: 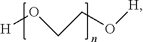
wherein n is 1-1000.

25. The cationic carrier unit of claim 24, wherein the n is at least about 110, at least about 111, at least about 112, at least about 113, at least about 114, at least about 115, at least about 116, at least about 117, at least about 118, at least about 119, at least about 120, at least about 121, at least about 122, at least about 123, at least about 124, at least about 125, at least about 126, at least about 127, at least about 128, at least about 129, at least about 130, at least about 131, at least about 132, at least about 133, at least about 134, at least about 135, at least about 136, at least about 137, at least about 138, at least about 139, at least about 140, or at least about 141.

26. The cationic carrier unit of claim 24, wherein the n is about 80 to about 90, about 90 to about 100, about 100 to about 110, about 110 to about 120, about 120 to about 130, about 140 to about 150, or about 150 to about 160.

27. The cationic carrier unit of any one of claims 17 to 26, wherein the water-soluble polymer is linear, branched, or dendritic.

28. The cationic carrier unit of any one of claims 1 to 27, wherein the cationic carrier moiety comprises one or more basic amino acids.

29. The cationic carrier unit of claim 28, wherein the cationic carrier moiety comprises at least about three, at least about four, at least about five, at least about six, at least about seven, at least about eight, at least about nine, at least about ten, at least about 11, at least about 12, at least about 13, at least about 14, at last 15, at least about 16, at least about 17, at least about 18, at least about 19, at least about 20, at least about 21, at least about 22, at least about 23, at least about 24, at least about 25, at least about 26, at least about 27, at least about 28, at least about 29, at least about 30, at least about 31, at least about 32, at least about 33, at least about 34, at least about 35, at least about 36, at least about 37, at least about 38, at least about 39, at least about 40, at least about 41, at least about 42, at least about 43, at least about 44, at least about 45, at least about 46, at least about 47, at least about 48, at least about 49, at least about 50, at least about 51, at least about 52, at least about 53, at least about 54, at least about 55, at least about 56, at least about 57, at least about 58, at least about 59, at least about 60, at least about 61, at least about 62, at least about 63, at least about 64, at least about 65, at least about 66, at least about 67, at least about 68, at least about 69, at least about 70, at least about 71, at least about 72, at least about 73, at least about 74, at least about 75, at least about 76, at least about 77, at least about 78, at least about 79, or at least about 80 basic amino acids.

30. The cationic carrier unit of claim 28, wherein the cationic carrier moiety comprises at least 20, at least 30, at least 40, at least 50, at least 60, at least about 70, at least about 80, at least about 90, at least about 100, at least about 110, at least about 120, at least about 130, at least about 140, or at least about 150 basic amino acids.

31. The cationic carrier unit of claim 28, wherein the cationic carrier moiety comprises about 10 to about 60, about 15 to about 60, about 20 to about 60, about 25 to about 60, about 30 to about 60, about 35 to about 60, about 40 to about 60, about 10 to about 55, about 15 to about 55, about 20 to about 55, about 25 to about 55, about 30 to about 55, about 35 to about 55, about 40 to about 55, about 10 to about 50, about 15 to about 50, about 20 to about 50, about 25 to about 50, about 30 to about 50, about 35 to about 50, about 40 to about 50, about 10 to about 45, about 15 to about 45, about 20 to about 45, about 25 to about 45, about 30 to about 45, about 35 to about 45, about 40 to about 45, about 10 to about 40, about 15 to about 40, about 20 to about 40, about 25 to about 40, about 30 to about 40, about 35 to about 40, about 10 to about 35, about 15 to about 35, about 20 to about 35, about 25 to about 35, about 30 to about 35, about 35 to about 35, about 40 to about 35, or about 40 to about 35 basic amino acids.

32. The cationic carrier unit of claim 28, wherein the cationic carrier moiety comprises about 10, about 20, about 30, about 40, about 50, or about 60 basic amino acids.

33. The cationic carrier unit of any one of claims 28 to 32, wherein the basic amino acid comprises arginine, lysine, histidine, or any combination thereof.

34. The cationic carrier unit of any one of claims 1 to 33, wherein the cationic carrier moiety comprises about 20, about 30, about 40, about 50, or about 60 lysines.

35. The cationic carrier unit of any one of claims 1 to 33, wherein the cationic carrier moity comprises about 32 lysines.

36. The cationic carrier unit of any one of claims 1 to 35, wherein the crosslinking moiety comprises one or more amino acids linked a crosslinking agent.

37. The cationic carrier unit of claim 36, wherein the crosslinking agent comprises a thiol group, a thiol derivative, or any combination thereof.

38. The cationic carrier unit of claim 36, wherein the crosslinking agent comprises a thiol group.

39. The cationic carrier unit of claims 36 to 38, wherein the amino acids in the crosslinking moiety comprise at least three, at least four, at least five, at least six, at least seven, at least eight, at least nine, at least ten, at least 11, at least 12, at least 13, at least 14, at last 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39 or at least 40 basic amino acids.

40. The cationic carrier unit of any one of claims 36 to 39, wherein the amino acids in the crosslinking moiety comprise about 1 to about 40, about 5 to about 40, about 10 to about 40, about 15 to about 40, about 20 to about 40, about 1 to about 35, about 5 to about 35, about 10 to about 35, about 15 to about 35, about 20 to about 35, about 1 to about 30, about 5 to about 30, about 10 to about 30, about 15 to about 30, about 20 to about 30, about 1 to about 25, about 5 to about 25, about 10 to about 25, about 15 to about 25, about 20 to about 25, about 1 to about 20, about 5 to about 20, about 10 to about 20, about 15 to about 20, about 1 to about 15, about 5 to about 15, about 10 to about 15, about 1 to about 10, about 5 to about 10, or about 1 to about 5 basic amino acids.

41. The cationic carrier unit of claims 36 to 40, wherein the amino acids in the crosslinking moiety comprise about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, or about 30 basic amino acids.

42. The cationic carrier unit of any one of claims 36 to 41, wherein the basic amino acids in the crosslinking moiety comprise arginine, lysine, histidine, or any combination thereof.

43. The cationic carrier unit of any one of claims 36 to 42, wherein the basic amino acids in the crosslinking moiety comprise about 5, about 10, about 11, about 12, about 13, about 14, about 15, about 16, about 17, about 18, about 19, about 20, about 25, about 30, or about 40 lysines.

44. The cationic carrier unit of any one of claims 36 to 43, wherein the basic amino acids in the crosslinking moiety comprise about 10 to about 20 lysines.

45. The cationic carrier unit of any one of claims 36 to 43, wherein the basic amino acids in the crosslinking moiety comprises about 16 lysines.

46. The cationic carrier unit of any one of claims 1 to 45, wherein the hydrophobic moiety is capable of modulating an immune response, an inflammatory response, and/or a tissue microenvironment.

47. The cationic carrier unit of claim 46, wherein the hydrophobic moiety is capable of modulating an immune response.

48. The cationic carrier unit of claim 46, wherein the hydrophobic moiety is capable of modulating a tumor microenvironment in a subject with a tumor.

49. The cationic carrier unit of claim 46, wherein the hydrophobic moiety is capable of inhibiting or reducing hypoxia in the tumor microenvironment.

50. The cationic carrier unit of claim 46, wherein the hydrophobic moiety is capable of inhibiting or reducing an inflammatory response.

51. The cationic carrier unit of claim 47, wherein the hydrophobic moiety is one or more amino acids linked to the nicotinic acid derivative.

52. The cationic carrier unit of claim 51, wherein the nicotinic acid derivative comprises: 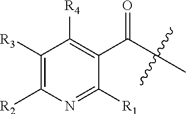
wherein at least one of R₁, R₂, R₃, and R₄ is an electron donating group.

53. The cationic carrier unit of claim 51, wherein the nicotinic acid derivative comprises: 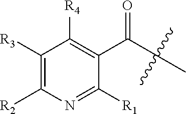
wherein at least one of R₁, R₂, R₃, and R₄ is an electron withdrawing group.

54. The cationic carrier unit of any one of claims 1 to 53, wherein the hydrophobic moiety comprises at least about two, at least about three, at least about four, at least about five, at least about six, at least about seven, at least about eight, at least about nine, at least about ten, at least about 11, at least about 12, at least about 13, at least about 14, at least about 15, at least about 16, at least about 17, at least about 18, at least about 19, at least about 20, at least about 21, at least about 22, at least about 23, at least about 24, at least about 25, at least about 26, at least about 27, at least about 28, at least about 29, at least about 30, at least about 31, at least about 32, at least about 33, at least about 34, at least about 35, at least about 36, at least about 37, at least about 38, at least about 39, at least about 40, at least about 41, at least about 42, at least about 43, at least about 44, at least about 45, at least about 46, at least about 47, at least about 48, at least about 49, at least about 50, at least about 51, at least about 52, at least about 53, at least about 54, at least about 55, at least about 56, at least about 57, at least about 58, at least about 59, at least about 60, at least about 61, at least about 62, at least about 63, at least about 64, at least about 65, at least about 66, at least about 67, at least about 68, at least about 69, at least about 70, at least about 71, at least about 72, at least about 73, at least about 74, at least about 75, at least about 76, at least about 77, at least about 78, at least about 79, or at least about 80 amino acids, each linked to a nicotinic acid derivative.

55. The cationic carrier unit of any one of claims 1 to 53, wherein the hydrophobic moiety comprises at least 20, at least 30, at least 40, at least 50, at least 60, at least about 70, at least about 80, at least about 90, at least about 100, at least about 110, at least about 120, at least about 130, at least about 140, or at least about 150 amino acids, each linked to a nicotinic acid derivative.

56. The cationic carrier unit of any one of claims 1 to 53, wherein the hydrophobic moiety comprises about 10 to about 60, about 15 to about 60, about 20 to about 60, about 25 to about 60, about 30 to about 60, about 35 to about 60, about 40 to about 60, about 10 to about 55, about 15 to about 55, about 20 to about 55, about 25 to about 55, about 30 to about 55, about 35 to about 55, about 40 to about 55, about 10 to about 50, about 15 to about 50, about 20 to about 50, about 25 to about 50, about 30 to about 50, about 35 to about 50, about 40 to about 50, about 10 to about 45, about 15 to about 45, about 20 to about 45, about 25 to about 45, about 30 to about 45, about 35 to about 45, about 40 to about 45, about 10 to about 40, about 15 to about 40, about 20 to about 40, about 25 to about 40, about 30 to about 40, about 35 to about 40, about 10 to about 35, about 15 to about 35, about 20 to about 35, about 25 to about 35, about 30 to about 35, about 35 to about 35, about 40 to about 35, or about 40 to about 35 amino acids, each linked to a nicotinic acid derivative.

57. The cationic carrier unit of any one of claims 1 to 53, wherein the hydrophobic moiety comprises about 10, about 20, about 30, about 40, about 50, or about 60 amino acids, each linked to a nicotinic acid derivative.

58. The cationic carrier unit of any one of claims 54 to 56, wherein the hydrophobic moiety comprises about 10 amino acids, about 20 amino acids, about 30 amino acids, about 40 amino acids, or about 50 amino acids, each linked to nicotinic acid derivatives.

59. The cationic carrier unit of claims 1 to 58, wherein the cationic carrier moiety comprises about 25 to about 40 lysines, the crosslinking moiety comprises about 10 to about 20 lysine-thiol, and the hydrophobic moiety comprises about 25 to about 40 lysine-nicotinic acid derivatives.

60. The cationic carrier unit of any one of claims 1 to 58, wherein the cationic carrier moiety comprises about 30 to about 35 lysines, the crosslinking moiety comprises about 13 to about 20 lysine-thiol, and the hydrophobic moiety comprises about 30 to about 35 lysine-nicotinic acid derivatives.

61. The cationic carrier unit of any one of claims 1 to 58, wherein the cationic carrier moiety comprises about 32 lysines, the crosslinking moiety comprises about 16 lysine-thiol, and the hydrophobic moiety comprises about 32 lysine-nicotinic acid derivatives.

62. The cationic carrier unit of claims 1 to 58, wherein the cationic carrier moiety comprises about 35 to about 60 lysines, the crosslinking moiety comprises about 5 to about 15 lysine-thiol, and the hydrophobic moiety comprises about 15 to about 30 lysine-nicotinic acid derivatives.

63. The cationic carrier unit of any one of claims 1 to 62, wherein the water-soluble biopolymer moiety comprises about 120 to about 130 PEG units.

64. The cationic carrier unit of any one of claims 1 to 63, further comprising a targeting moiety (TM).

65. The cationic carrier unit of claim 64, wherein the targeting moiety is capable of targeting a tissue.

66. The cationic carrier unit of claim 65 wherein the tissue is liver, brain, kidney, lung, ovary, pancreas, thyroid, breast, stomach, or any combination thereof.

67. The cationic carrier unit of claim 65, wherein the targeting moiety is capable of being transported by large neutral amino acid transporter 1 (LAT1).

68. The cationic carrier unit of claim 67, wherein the targeting moiety is an amino acid.

69. The cationic carrier unit of claim 67, wherein the targeting moiety comprises a branched-chain or aromatic amino acid.

70. The cationic carrier unit of claim 68, wherein the targeting moiety is phenylalanine, valine, leucine, and/or isoleucine.

71. The cationic carrier unit of claim 70, wherein the amino acid is phenylalanine.

72. The cationic carrier unit of any one of claims 64 to 71, wherein the targeting moiety is linked to the water-soluble polymer.

73. The cationic carrier unit of claim 71, wherein the targeting moiety is linked to the water-soluble polymer by a linker.

74. A micelle comprising the cationic carrier unit of any one of claims 1 to 73 and an anionic payload, wherein the cationic carrier moiety of the cationic carrier complex and the anionic payload are associated with each other.

75. The micelle of claim 74, wherein the association is a covalent bond.

76. The micelle of claim 74, wherein the association is a non-covalent bond.

77. The micelle of claim 74, wherein the association is an ionic bond.

78. The micelle of any one of claims 74 to 77, wherein the positive charge of the cationic carrier moiety of the cationic carrier unit is sufficient to form a micelle when mixed with an anionic payload in a solution, wherein the overall ionic ratio of the positive charges of the cationic carrier moiety of the cationic carrier unit and the negative charges of the anionic payload in the solution is between about 1:3 and about 3:1.

79. The micelle of claim 78, wherein the positive charge of the cationic carrier moiety of the cationic carrier unit is sufficient to form a micelle when mixed with an anionic payload in a solution, wherein the overall ionic ratio of the positive charges of the cationic carrier moiety of the cationic carrier unit and the negative charges of the anionic payload in the solution is about 1:3, about 1:2.5; about 1:2, about 1:1.5; about 1:1, about 1:0.5; about 0.5:1, about 1.5:1; about 2:1, about 2.5:1, or about 3:1.

80. The micelle of claim 78, wherein the positive charge of the cationic carrier moiety of the cationic carrier unit is sufficient to form a micelle when mixed with an anionic payload in a solution, wherein the overall ionic ratio of the positive charges of the cationic carrier moiety of the cationic carrier unit and the negative charges of the anionic payload in the solution is about 1:1.

81. The micelle of any one of claims 74 to 80, wherein the cationic carrier unit is capable of protecting the anionic payload from degradation by a DNase and/or an RNase.

82. The micelle of any one of claims 74 to 81, wherein the anionic payload is not conjugated to the cationic carrier unit by a covalent bond and/or the anionic payload interacts with the cationic carrier moiety of the cationic carrier unit only via an ionic interaction.

83. The micelle of any one of claims 74 to 82, wherein the half-life of the anionic payload is extended compared to the half-life of a free anionic payload not incorporated into a micelle.

84. The micelle of claim 74, wherein the positive charges of the cationic carrier moiety of the cationic carrier unit and the negative charges of the anionic payload in the micelle are at an ionic ratio of about 2:1, about 1.9:1, about 1.8:1, about 1.7:1, about 1.6:1, about 1.5:1, about 1.4:1, about 1.3:1, about 1.2:1, about 1:1, about 1:1.1, about 1:1.2, about 1:1.3, about 1:1.4, about 1:1.5, about 1:1.6, about 1:1.7, about 1:1.8, about 1:1.9, or about 1:2.

85. The micelle of claim 74, wherein the positive charges of the cationic carrier moiety of the cationic carrier unit and the negative charges of the anionic payload in the micelle are at an ionic ratio of about 3:1, about 2.9:1, about 2.8:1, about 2.7:1, about 2.6:1, about 2.5:1, about 2.4:1, about 2.3:1, about 2.2:1, about 2.1:1, about 1:2.1, about 1:2.2, about 1:2.3, about 1:2.4, about 1:2.5, about 1:2.6, about 1:2.7, about 1:2.8, about 1:2.9, or about 1:3.

86. The micelle of any one of claims 74 to 85, where the diameter of the micelle is between about 1 nm and 100 nm, between about 10 nm and about 100 nm, between about 10 nm and about 90 nm, between about 10 nm and about 80 nm, between about 10 nm and about 70 nm, between about 20 nm and about 100 nm, between about 20 nm and about 90 nm, between about 20 nm and about 80 nm, between about 20 nm and about 70 nm, between about 30 nm and about 100 nm, between about 30 nm and about 90 nm, between about 30 nm and about 80 nm, between about 30 nm and about 70 nm, between about 40 nm and about 100 nm, between about 40 nm and about 90 nm, between about 40 nm and about 80 nm, or between about 40 nm and about 70 nm.

87. The micelle of any one of claims 74 to 86, wherein the anionic payload comprises a nucleic acid.

88. The micelle of claim 87, wherein the nucleic acid comprises mRNA, miRNA, miRNA sponge, tough decoy miRNA, antimir, small RNA, rRNA, siRNA, shRNA, gDNA, cDNA, pDNA, PNA, BNA, antisense oligonucleotide (ASO), aptamer, cyclic dinucleotide, or any combination thereof.

89. The micelle of claim 87 or 88, wherein the nucleic acid comprises at least one nucleoside analog.

90. The micelle of claim 89, wherein the nucleoside analog comprises Locked Nucleic Acid (LNA); 2′—O-alkyl-RNA; 2′-amino-DNA; 2′-fluoro-DNA; arabino nucleic acid (ANA); 2′-fluoro-ANA, hexitol nucleic acid (HNA), intercalating nucleic acid (INA), constrained ethyl nucleoside (cEt), 2′—O-methyl nucleic acid (2′—OMe), 2′—O-methoxyethyl nucleic acid (2′-MOE), or any combination thereof.

91. The micelle of any one of claims 87 to 90, wherein the nucleic acid comprises a nucleotide sequence having 5 to 30 nucleotides in length.

92. The micelle of claim 91, wherein the nucleotide sequence is 7, 8, 9, 10, 11, 12, 13, 14, 15, 16, 17, 18, 19, 20, 21, 22, 23, 24, 25, or 26 nucleotides in length.

93. The micelle of claim 91 or 92, wherein the nucleotide sequence has a backbone, which comprises a phosphodiester linkage, a phosphotriester linkage, a methylphosphonate linkage, a phosphoramidate linkage, a phosphorothioate linkage, and combinations thereof.

94. The micelle of claim 87, wherein the nucleic acid comprises the nucleotide sequence as set forth in SEQ ID NO: 18 (miR-485 3p inhibitor).

95. A composition comprising the cationic carrier unit of any one of claims 1 to 73 and an anionic payload.

96. A pharmaceutical composition comprising the cationic carrier unit of any one of claims 1 to 73, the micelle of any one of claims 74 to 94, the composition of claim 95, and a pharmaceutically acceptable carrier.

97. A method of preparing the cationic carrier unit of any one of claims 1 to 73 comprising linking the cationic carrier moiety to the crosslinking moiety and the hydrophobic moiety.

98. The method of claim 97, further linking a water-soluable polymer and a targeting moiety.

99. The method of preparing the micelle of any one of claims 74 to 94, comprising mixing the cationic carrier unit with the anionic payload at an ionic ratio of 1:1 in solution.

100. The method of preparing the micelle of any one of claims 74 to 94, comprising mixing the cationic carrier unit with the anionic payload at an ionic ratio of 2:1 in solution.

101. The method of preparing the micelle of any one of claims 74 to 94, comprising mixing the cationic carrier unit with the anionic payload at an ionic ratio between about 1:3 and about 3:1 in solution.

102. The method of claim 101, further comprising purifying the micelle.

103. A method of treating a disease or condition in a subject in need thereof comprising administering the micelle of any one of claims 74 to 94 or the pharmaceutical composition of any one of claim 95 to the subject.

104. The method of claim 103, wherein the anionic payload in the core of the micelle exhibits a longer half-life than a corresponding anionic payload not integrated into a micelle.

105. The method of claim 103 or 104, wherein the subject is a mammal.

106. A method to reduce inflammation in a subject suffering from a neurodegenerative disease comprising administering a therapeutically effective amount of a micelle of any one of claims 74 to 94 to the subject.

107. A method to recover and/or induce neurogenesis in a subject suffering from a neurodegenerative disease comprising administering a therapeutically effective amount of a micelle of any one of claims 74 to 94 to the subject.

108. A method to improve cognitive function in a subject suffering from a neurodegenerative disease comprising administering a therapeutically effective amount of a micelle of any one of claims 74 to 94 to the subject.

109. The method of any one of claims 103 to 108, wherein the neurodegenerative disease is Alzheimer's disease.

110. A method to reduce amyloid plaque burden in a subject suffering from Alzheimer's disease comprising administering a therapeutically effective amount of a micelle of any one of claims 74 to 94 to the subject.


SEQUENCE LISTING (80 sequences)

SEQ ID NO: 1 - protein, 4 aa, synthetic construct
  REPEAT 1: note = Wherein Gly can be repeated between 1 to 4 times
  REGION 1..2: note = Linker
  REPEAT 1..2: note = Wherein the amino acids herein can be repeated 1 to 49 times.
  VARIANT 3..4: note = Wherein the amino acids can be present or not present
  VARIANT 2: note = Wherein Ser can be present or not present
  REPEAT 3: note = Wherein Gly can be repeated between 1 to 4 times
  VARIANT 4: note = Wherein Ser can be present or not present
GSGS

SEQ ID NO: 2 - protein, 4 aa, synthetic construct
  REGION 1..4: note = Linker
GGGG

SEQ ID NO: 3 - protein, 4 aa, synthetic construct
  REGION 1..2: note = Linker
  REPEAT 1..2: note = Wherein the amino acids may be repeated between 1 and 99 times.
  VARIANT 3..4: note = Wherein the amino acids can be present or not present
GAGA

SEQ ID NO: 4 - protein, 6 aa, synthetic construct
  REGION 1..6: note = Linker
  REPEAT 1..3: note = Wherein the amino acids may be repeated between 1 and 99 times.
  VARIANT 4..6: note = Wherein the amino acids can be present or not present
GGSGGS

SEQ ID NO: 5 - protein, 4 aa, synthetic construct
  REGION 1..4: note = Linker
  REPEAT 1..4: note = Wherein the amino acids may be repeated between 1 to 100 times.
GGGS

SEQ ID NO: 6 - protein, 8 aa, synthetic construct
  REGION 1..8: note = Linker
  REPEAT 1..3: note = Wherein the amino acids may be repeated between 1 to 100 times.
  REPEAT 4..8: note = Wherein the amino acids may be repeated between 1 to 100 times.
GGSGGGGS

SEQ ID NO: 7 - protein, 7 aa, synthetic construct
  REGION 1..7: note = Linker
SGGSGGS

SEQ ID NO: 8 - protein, 15 aa, synthetic construct
  REGION 1..15: note = Linker
GGSGGSGGSGGSGGG

SEQ ID NO: 9 - protein, 16 aa, synthetic construct
  REGION 1..16: note = Linker
GGSGGSGGGGSGGGGS

SEQ ID NO: 10 - protein, 18 aa, synthetic construct
  REGION 1..18: note = Linker
GGSGGSGGSGGSGGSGGS

SEQ ID NO: 11 - protein, 15 aa, synthetic construct
  REGION 1..15: note = Linker
GGGGSGGGGSGGGGS

SEQ ID NO: 12 - protein, 4 aa, synthetic construct
  REGION 1..4: note = Linker
  REPEAT 1..4: note = Wherein the amino acids may be repeated between 1 to 100 times.
GGGG

SEQ ID NO: 13 - RNA, 22 nt, synthetic construct
  source 1..22: mol_type = other RNA
  misc_feature 1..22: note = human miR-204-5p
ttccctttgtcatcctatgcct

SEQ ID NO: 14 - RNA, 22 nt, synthetic construct
  source 1..22: mol_type = other RNA
  misc_feature 1..22: note = human miR-132-3p
taacagtctacagccatggtcg

SEQ ID NO: 15 - RNA, 22 nt, synthetic construct
  source 1..22: mol_type = other RNA
  misc_feature 1..22: note = Antisense Oligonucleotide
aggcataggatgacaaagggaa

SEQ ID NO: 16 - RNA, 22 nt, synthetic construct
  source 1..22: mol_type = other RNA
  misc_feature 1..22: note = Antisense Oligonucleotide
cgaccatggctgtagactgtta

SEQ ID NO: 17 - RNA, 22 nt, synthetic construct
  source 1..22: mol_type = other RNA
  misc_feature 1..22: note = human miR-485-3p
gtcatacacggctctcctctct

SEQ ID NO: 18 - RNA, 22 nt, synthetic construct
  source 1..22: mol_type = other RNA
  misc_feature 1..22: note = Antisense Oligonucleotide
agagaggagagccgtgtatgac

SEQ ID NO: 19-25 - skipped (7 entries)

SEQ ID NO: 26 - RNA, 10 nt, synthetic construct
  source 1..10: mol_type = other RNA
  misc_feature 1..10: note = Antisense Oligonucleotide
ccgtgtatga

SEQ ID NO: 27 - RNA, 11 nt, synthetic construct
  source 1..11: mol_type = other RNA
gccgtgtatga

SEQ ID NO: 28 - RNA, 12 nt, synthetic construct
  source 1..12: mol_type = other RNA
  misc_feature 1..12: note = Antisense Oligonucleotide
agccgtgtatga

SEQ ID NO: 29 - RNA, 13 nt, synthetic construct
  source 1..13: mol_type = other RNA
  misc_feature 1..13: note = Antisense Oligonucleotide
gagccgtgtatga

SEQ ID NO: 30 - RNA, 14 nt, synthetic construct
  source 1..14: mol_type = other RNA
  misc_feature 1..14: note = Antisense Oligonucleotide
agagccgtgtatga

SEQ ID NO: 31 - RNA, 15 nt, synthetic construct
  source 1..15: mol_type = other RNA
  misc_feature 1..15: note = Antisense Oligonucleotide
gagagccgtgtatga

SEQ ID NO: 32 - RNA, 16 nt, synthetic construct
  source 1..16: mol_type = other RNA
  misc_feature 1..16: note = Antisense Oligonucleotide
ggagagccgtgtatga

SEQ ID NO: 33 - RNA, 17 nt, synthetic construct
  source 1..17: mol_type = other RNA
  misc_feature 1..17: note = Antisense Oligonucleotide
aggagagccgtgtatga

SEQ ID NO: 34 - RNA, 18 nt, synthetic construct
  source 1..18: mol_type = other RNA
  misc_feature 1..18: note = Antisense Oligonucleotide
gaggagagccgtgtatga

SEQ ID NO: 35 - RNA, 19 nt, synthetic construct
  source 1..19: mol_type = other RNA
  misc_feature 1..19: note = Antisense Oligonucleotide
agaggagagccgtgtatga

SEQ ID NO: 36 - RNA, 20 nt, synthetic construct
  source 1..20: mol_type = other RNA
  misc_feature 1..20: note = Antisense Oligonucleotide
gagaggagagccgtgtatga

SEQ ID NO: 37-38 - skipped (2 entries)

SEQ ID NO: 39 - RNA, 10 nt, synthetic construct
  source 1..10: mol_type = other RNA
cgtgtatgac

SEQ ID NO: 40 - RNA, 11 nt, synthetic construct
  source 1..11: mol_type = other RNA
  misc_feature 1..11: note = Antisense Oligonucleotide
ccgtgtatgac

SEQ ID NO: 41 - RNA, 12 nt, synthetic construct
  source 1..12: mol_type = other RNA
  misc_feature 1..12: note = Antisense Oligonucleotide
gccgtgtatgac

SEQ ID NO: 42 - RNA, 13 nt, synthetic construct
  source 1..13: mol_type = other RNA
  misc_feature 1..13: note = Antisense Oligonucleotide
agccgtgtatgac

SEQ ID NO: 43 - RNA, 14 nt, synthetic construct
  source 1..14: mol_type = other RNA
  misc_feature 1..14: note = Antisense Oligonucleotide
gagccgtgtatgac

SEQ ID NO: 44 - RNA, 15 nt, synthetic construct
  source 1..15: mol_type = other RNA
  misc_feature 1..15: note = Antisense Oligonucleotide
agagccgtgtatgac

SEQ ID NO: 45 - RNA, 16 nt, synthetic construct
  source 1..16: mol_type = other RNA
  misc_feature 1..16: note = Antisense Oligonucleotide
gagagccgtgtatgac

SEQ ID NO: 46 - RNA, 17 nt, synthetic construct
  source 1..17: mol_type = other RNA
  misc_feature 1..17: note = Antisense Oligonucleotide
ggagagccgtgtatgac

SEQ ID NO: 47 - RNA, 18 nt, synthetic construct
  source 1..18: mol_type = other RNA
  misc_feature 1..18: note = Antisense Oligonucleotide
aggagagccgtgtatgac

SEQ ID NO: 48 - RNA, 19 nt, synthetic construct
  source 1..19: mol_type = other RNA
  misc_feature 1..19: note = Antisense Oligonucleotide
gaggagagccgtgtatgac

SEQ ID NO: 49 - RNA, 20 nt, synthetic construct
  source 1..20: mol_type = other RNA
  misc_feature 1..20: note = Antisense Oligonucleotide
agaggagagccgtgtatgac

SEQ ID NO: 50 - RNA, 21 nt, synthetic construct
  source 1..21: mol_type = other RNA
  misc_feature 1..21: note = Antisense Oligonucleotide
gagaggagagccgtgtatgac

SEQ ID NO: 51-53 - skipped (3 entries)

SEQ ID NO: 54 - DNA, 10 bp, synthetic construct
  source 1..10: mol_type = other DNA
  misc_feature 1..10: note = Antisense Oligonucleotide
ccgtgtatga

SEQ ID NO: 55 - DNA, 11 bp, synthetic construct
  source 1..11: mol_type = other DNA
  misc_feature 1..11: note = Antisense Oligonucleotide
gccgtgtatga

SEQ ID NO: 56 - DNA, 12 bp, synthetic construct
  source 1..12: mol_type = other DNA
  misc_feature 1..12: note = Antisense Oligonucleotide
agccgtgtatga

SEQ ID NO: 57 - DNA, 13 bp, synthetic construct
  source 1..13: mol_type = other DNA
  misc_feature 1..13: note = Antisense Oligonucleotide
gagccgtgtatga

SEQ ID NO: 58 - DNA, 14 bp, synthetic construct
  source 1..14: mol_type = other DNA
  misc_feature 1..14: note = Antisense Oligonucleotide
agagccgtgtatga

SEQ ID NO: 59 - DNA, 15 bp, synthetic construct
  source 1..15: mol_type = other DNA
  misc_feature 1..15: note = Antisense Oligonucleotide
gagagccgtgtatga

SEQ ID NO: 60 - DNA, 16 bp, synthetic construct
  source 1..16: mol_type = other DNA
  misc_feature 1..16: note = Antisense Oligonucleotide
ggagagccgtgtatga

SEQ ID NO: 61 - DNA, 17 bp, synthetic construct
  source 1..17: mol_type = other DNA
  misc_feature 1..17: note = Antisense Oligonucleotide
aggagagccgtgtatga

SEQ ID NO: 62 - DNA, 18 bp, synthetic construct
  source 1..18: mol_type = other DNA
  misc_feature 1..18: note = Antisense Oligonucleotide
gaggagagccgtgtatga

SEQ ID NO: 63 - DNA, 19 bp, synthetic construct
  source 1..19: mol_type = other DNA
  misc_feature 1..19: note = Antisense Oligonucleotide
agaggagagccgtgtatga

SEQ ID NO: 64 - DNA, 20 bp, synthetic construct
  source 1..20: mol_type = other DNA
  misc_feature 1..20: note = Antisense Oligonucleotide
gagaggagagccgtgtatga

SEQ ID NO: 65-66 - skipped (2 entries)

SEQ ID NO: 67 - DNA, 10 bp, synthetic construct
  source 1..10: mol_type = other DNA
  misc_feature 1..10: note = Antisense Oligonucleotide
cgtgtatgac

SEQ ID NO: 68 - DNA, 11 bp, synthetic construct
  source 1..11: mol_type = other DNA
  misc_feature 1..11: note = Antisense Oligonucleotide
ccgtgtatgac

SEQ ID NO: 69 - DNA, 12 bp, synthetic construct
  source 1..12: mol_type = other DNA
  misc_feature 1..12: note = Antisense Oligonucleotide
gccgtgtatgac

SEQ ID NO: 70 - DNA, 13 bp, synthetic construct
  source 1..13: mol_type = other DNA
  misc_feature 1..13: note = Antisense Oligonucleotide
agccgtgtatgac

SEQ ID NO: 71 - DNA, 14 bp, synthetic construct
  source 1..14: mol_type = other DNA
  misc_feature 1..14: note = Antisense Oligonucleotide
gagccgtgtatgac

SEQ ID NO: 72 - DNA, 15 bp, synthetic construct
  source 1..15: mol_type = other DNA
  misc_feature 1..15: note = Antisense Oligonucleotide
agagccgtgtatgac

SEQ ID NO: 73 - DNA, 16 bp, synthetic construct
  source 1..16: mol_type = other DNA
  misc_feature 1..16: note = Antisense Oligonucleotide
gagagccgtgtatgac

SEQ ID NO: 74 - DNA, 17 bp, synthetic construct
  source 1..17: mol_type = other DNA
  misc_feature 1..17: note = Antisense Oligonucleotide
ggagagccgtgtatgac

SEQ ID NO: 75 - DNA, 18 bp, synthetic construct
  source 1..18: mol_type = other DNA
  misc_feature 1..18: note = Antisense Oligonucleotide
aggagagccgtgtatgac

SEQ ID NO: 76 - DNA, 19 bp, synthetic construct
  source 1..19: mol_type = other DNA
  misc_feature 1..19: note = Antisense Oligonucleotide
gaggagagccgtgtatgac

SEQ ID NO: 77 - DNA, 20 bp, synthetic construct
  source 1..20: mol_type = other DNA
  misc_feature 1..20: note = Antisense Oligonucleotide
agaggagagccgtgtatgac

SEQ ID NO: 78 - DNA, 21 bp, synthetic construct
  source 1..21: mol_type = other DNA
  misc_feature 1..21: note = Antisense Oligonucleotide
gagaggagagccgtgtatgac

SEQ ID NO: 79 - protein, 4 aa, synthetic construct
  REGION 1: note = Linker
  REPEAT 1: note = Wherein Gly may be repeated 1 to 97 times
  VARIANT 2: note = Wherein Gly can be present or not present
  VARIANT 3: note = Wherein Gly can be present or not present
  VARIANT 4: note = Wherein Gly can be present or not present
GGGG

SEQ ID NO: 80 - protein, 4 aa, synthetic construct
  REGION 1..2: note = Linker
  REPEAT 1: note = Wherein Gly can be repeated between 1 and 100 times
  REPEAT 1..2: note = Wherein the amino acids may be repeated between 1 to 99 times
  VARIANT 3..4: note = Wherein the amino acids can be present or not present
  REPEAT 3: note = Wherein Gly can be repeated between 1 and 100 times
GSGS